(19) United States
(12) Patent Application Publication
(10) Pub. No.: US 2009/0253718 A1
(43) Pub. Date: Oct. 8, 2009

(54) Pharmaceutical Compounds

(75) Inventors: Thomas Glanmor Davies, Cambridge (GB); Michelle Dawn Garrett, Surrey (GB); Robert George Boyle, Cambridge (GB); Ian Collins, Redhill (GB)

Correspondence Address: HESLIN ROTHENBERG FARLEY & MESITI PC, 5 COLUMBIA CIRCLE, ALBANY, NY, 12203, US

(73) Assignees: Astex Therapeutics Limited, Cambridge (GB); The Institute of Cancer Research: Royal Cancer Hospital, London (GB); Cancer Research Technology Limited, London (GB)

(21) Appl. No.: 12/298,117

(22) PCT Filed: Apr. 25, 2007
(86) PCT No.: PCT/GB07/01510

§ 371 Date: Oct. 22, 2008

(30) Foreign Application Priority Data
Apr. 25, 2006 (GB) 0608175.6
Apr. 25, 2006 (GB) 0608185.5

(51) Int. Cl. A61K 31/522; A61K 31/52; A61P 35/04; A61P 35/00; A61K 31/519

(52) U.S. Cl. 514/263.22; 514/263.4; 514/263.1; 514/263.23; 514/262.1; 514/265.1; 514/263.2

(57) ABSTRACT

The invention provides a compound having the formula (I):

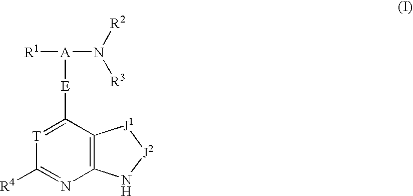

or salts, solvates, tautomers or N-oxides thereof, wherein T is N or CR⁵; J¹-J² is N═C(R⁶), (R⁷)C═N, (R⁸)N—C(O), (R⁸)₂C—C(O), N═N or (R⁷)C═C(R⁶); A is an optionally substituted saturated C₁₋₇ hydrocarbon linker group having a maximum chain length of 5 atoms extending between R¹ and NR²R³ and a maximum chain length of 4 atoms extending between E and NR²R³, one of the carbon atoms in the linker group being optionally replaced by oxygen or nitrogen; E is a monocyclic or bicyclic carbocyclic or heterocyclic group or an acyclic group X-G wherein X is CH₂, O, S or NH and G is a C₁₋₄ alkylene chain wherein one of the carbon atoms is optionally replaced by O, S or NH; R¹ is hydrogen or an aryl or heteroaryl group; R² and R³ are each hydrogen, optionally substituted C₁₋₄ hydrocarbyl or optionally substituted C₁₋₄ acyl; or NR²R³ forms an imidazole group or a saturated monocyclic heterocyclic group having 4-7 ring members; or NR²R³ and A together form a saturated monocyclic heterocyclic group having 4-7 ring members which is optionally substituted by C₁₋₄ alkyl; or NR²R³ and the adjacent carbon atom of linker group A together form a cyano group; or R¹, A and NR²R³ together form a cyano group; and R⁴, R⁵, R⁶, R⁷ and R⁸ are each independently selected from hydrogen and various substituents as defined in the claims, wherein the compound is for use in: (a) the treatment or prophylaxis of a disease or condition in which the modulation (e.g. inhibition) of ROCK kinase or protein kinase p70S6K is indicated; and/or (b) the treatment of a subject or patient population in which the modulation (e.g. inhibition) of ROCK kinase or protein kinase p70S6K is indicated.

### TECHNICAL FIELD

This invention relates to the use of purine, purinone and deazapurine and deazapurinone compounds in: (a) the treatment or prophylaxis of a disease or condition in which the modulation (e.g. inhibition) of ROCK kinase is indicated; and/or (b) the treatment of a subject or patient population in which the modulation (e.g. inhibition) of ROCK kinase is indicated; and/or (c) the treatment or prophylaxis of a disease or condition in which the modulation (e.g. inhibition) of protein kinase p70S6K is indicated; and/or (d) the treatment of a subject or patient population in which the modulation (e.g. inhibition) of protein kinase p70S6K is indicated. The invention also relates to said compounds for said uses and to various pharmaceutical compositions containing the purine, purinone and deazapurine and deazapurinone compounds.

### BACKGROUND OF THE INVENTION

### Protein Kinases

Protein kinases constitute a large family of structurally related enzymes that are responsible for the control of a wide variety of signal transduction processes within the cell (Hardie, G. and Hanks, S. (1995) The Protein Kinase Facts Book. I and II, Academic Press, San Diego, Calif.). The kinases may be categorized into families by the substrates they phosphorylate (e.g., protein-tyrosine, protein-serine/threonine, lipids, etc.). Sequence motifs have been identified that generally correspond to each of these kinase families (e.g., Hanks, S. K., Hunter, T., FASEB J., 9:576-596 (1995); Knighton, et al., Science, 253:407-414 (1991); Hiles, et al., Cell, 70:419-429 (1992); Kunz, et al., Cell, 73:585-596 (1993); Garcia-Bustos, et al., EMBO J., 13:2352-2361 (1994)).

Protein kinases may be characterized by their regulation mechanisms. These mechanisms include, for example, autophosphorylation, transphosphorylation by other kinases, protein-protein interactions, protein-lipid interactions, and protein-polynucleotide interactions. An individual protein kinase may be regulated by more than one mechanism.

Kinases regulate many different cell processes including, but not limited to, proliferation, differentiation, apoptosis, motility, transcription, translation and other signalling processes, by adding phosphate groups to target proteins. These phosphorylation events act as molecular on/off switches that can modulate or regulate the target protein biological function. Phosphorylation of target proteins occurs in response to a variety of extracellular signals (hormones, neurotransmitters, growth and differentiation factors, etc.), cell cycle events, environmental or nutritional stresses, etc. The appropriate protein kinase functions in signalling pathways to activate or inactivate (either directly or indirectly), for example, a metabolic enzyme, regulatory protein, receptor, cytoskeletal protein, ion channel or pump, or transcription factor. Uncontrolled signalling due to defective control of protein phosphorylation has been implicated in a number of diseases, including, for example, inflammation, cancer, allergy/asthma, diseases and conditions of the immune system, diseases and conditions of the central nervous system, and angiogenesis.

Apoptosis or programmed cell death is an important physiological process which removes cells no longer required by an organism. The process is important in early embryonic growth and development allowing the non-necrotic controlled breakdown, removal and recovery of cellular components. The removal of cells by apoptosis is also important in the maintenance of chromosomal and genomic integrity of growing cell populations. There are several known checkpoints in the cell growth cycle at which DNA damage and genomic integrity are carefully monitored. The response to the detection of anomalies at such checkpoints is to arrest the growth of such cells and initiate repair processes. If the damage or anomalies cannot be repaired then apoptosis is initiated by the damaged cell in order to prevent the propagation of faults and errors. Cancerous cells consistently contain numerous mutations, errors or rearrangements in their chromosomal DNA. It is widely believed that this occurs in part because the majority of tumours have a defect in one or more of the processes responsible for initiation of the apoptotic process. Normal control mechanisms cannot kill the cancerous cells and the chromosomal or DNA coding errors continue to be propagated. As a consequence restoring these pro-apoptotic signals or suppressing unregulated survival signals is an attractive means of treating cancer.

The signal transduction pathway containing the enzymes phosphatidylinositol 3-kinase (PI3K), PDK1 and PKB amongst others, has long been known to mediate increased resistance to apoptosis or survival responses in many cells. There is a substantial amount of data to indicate that this pathway is an important survival pathway used by many growth factors to suppress apoptosis. The enzymes of the PI3K family are activated by a range of growth and survival factors e.g. EGF, PDGF and through the generation of polyphosphatidylinositols, initiates the activation of the downstream signalling events including the activity of the kinases PDK1 and protein kinase B (PKB) also known as akt. This is also true in host tissues, e.g. vascular endothelial cells as well as neoplasias.

### Protein Kinase P70S6K

The 70 kDa ribosomal protein kinase p70S6K (also known as SK6, p70/p85 S6 kinase, p70/p85 ribosomal S6 kinase and pp70s6k) is a member of the AGC subfamily of protein kinases. p70S6K is a serine-threonine kinase that is a component of the phosphatidylinositol 3 kinase (Pl3K)/AKT pathway. p70S6K is downstream of PI3K, and activation occurs through phosphorylation at a number of sites in response to numerous mitogens, hormones and growth factors. This response may be under the control of mTOR since rapamycin acts to inhibit p70S6K activity and blocks protein synthesis, specifically as a result of a down-regulation of translation of these mRNA's encoding ribosomal proteins. p70S6K is also regulated by PI3K and its downstream target AKT. Wortmannin and rapamycin cause a decrease in p70S6K phosphorylation at sites dependent of the PI3K pathway. Mutant p70S6K is inhibited by wortmannin but not by rapamycin suggesting that the PI3K pathway can exhibit effects on p70S6K independent of the regulation of mTOR activity.

The enzyme p70S6K modulates protein synthesis by phosphorylation of the S6 ribosomal protein. S6 phosphorylation correlates with increased translation of mRNAs encoding components of the translational apparatus, including ribosomal proteins and translational elongation factors whose increased expression is essential for cell growth and proliferation. These mRNAs contain an oligopyrimidime tract at their 5′ transcriptional start (termed 5′TOP), which has been shown to be essential for their regulation at the translational level.

In addition to its involvement in translation, p70S6K activation has also been implicated in cell cycle control, neuronal cell differentiation, regulation of cell motility and a cellular response that is important in tumor metastases, the immune response and tissue repair. Antibodies to p70S6K abolish the mitogenic response driven entry of rat fibroblasts into S phase, indication that p70S6K function is essential for the progression from G1 to S phase in the cell cycle. Furthermore inhibition of cell cycle proliferation at the G1 to S phase of the cell cycle by rapamycin has been identified as a consequence of inhibition of the production of the hyperphosphorylated, activated form of p70S6K.

The tumor suppressor LKB1 activates AMPK which phosphorylates the TSC1/2 complex in the mTOR/p70S6K pathway, therefore feeds into p70S6K through a PKB independent pathway. Mutations in LKB1 cause Peutz-Jeghers syndrome (PJS), where patients with PJS are 15 times more likely to develop cancer than the general population. In addition, ⅓ of lung adenocarcinomas harbor inactivating LKB1 mutations.

A role for p70S6K in tumor cell proliferation and protection of cells from apoptosis is supported based on its participation in growth factor receptor signal transduction, overexpression and activation in tumor tissues. For example, Northern and Western analyses revealed that amplification of the PS6K gene was accompanied by corresponding increases in mRNA and protein expression, respectively (Cancer Res. (1999) 59: 1408-11—Localization of PS6K to Chromosomal Region 17q23 and Determination of Its Amplification in Breast Cancer).

Chromosome 17q23 is amplified in up to 20% of primary breast tumors, in 87% of breast tumors containing BRCA2 mutations and in 50% of tumors containing BRCA1 mutations, as well as other cancer types such as pancreatic, bladder and neuroblastoma (see M Barlund, O Monni, J Kononen, R Cornelison, J Torhorst, G Sauter, O-P Kallioniemi and Kallioniemi A, Cancer Res., 2000, 60:5340-5346). It has been shown that 17q23 amplifications in breast cancer involve the PAT1, RAD51C, PS6K, and SIGMA1B genes (Cancer Res. (2000): 60, pp. 5371-5375).

The p70S6K gene has been identified as a target of amplification and overexpression in this region, and statistically significant association between amplification and poor prognosis has been observed.

Clinical inhibition of p70S6K activation was observed in renal carcinoma patients treated with CCI-779 (rapamycin ester), an inhibitor of the upstream kinase mTOR. A significant linear association between disease progression and inhibition of p70S6K activity was reported.

p70S6K has been implicated in metabolic diseases and disorders. It was reported that the absence of p70S6 protects against age- and diet-induced obesity while enhancing insulin sensitivity. A role for p70S6K in metabolic diseases and disorders such as obesity, diabetes, metabolic syndrome, insulin resistance, hyperglycemia, hyperaminoacidemia, and hyperlipidmia is supported based upon the findings.

### ROCK Kinases

The ROCK kinase family comprises two known members: ROCK1 and ROCK2:
ROCK1. Synonyms: Rho-associated protein kinase 1; p160 ROCK; P160 ROK; p160 ROCK-1, Rho-associated, coiled-coil containing protein kinase 1; Rho kinase 1; ROK beta.
ROCK2. Synonyms: Rho-associated protein kinase 2; p164 ROCK; p164 ROK; p164 ROCK-2; Rho-associated, coiled-coil containing protein kinase 2, Rho kinase 2; ROK alpha.

The process of metastasis involves a restructuring of the cytoskeleton as well as cell-cell and cell-matrix adhesions allowing cells to break away from the tumor mass, invade local tissue, and ultimately spread throughout the body. These effects on cell morphology and adhesion are regulated by members of the Rho GTPase family.

Activated RhoA is capable of interacting with several effecter proteins including the ROCK kinases ROCK1 and ROCK2. ROCK1 and ROCK2 can be activated by the RhoA-GTP complex via physical association. Activated ROCKs phosphorylate a number of substrates and play important roles in pivotal cellular functions. The substrates for ROCKs include myosin binding subunit of myosin light chain phosphatase (MBS, also named MYPT1), adducin, moesin, myosin light chain (MLC), LIM kinase, and the transcription factor FHL. The phosphorylation of theses substrates modulate the biological activity of the proteins and provide a means to alter a cell's response to external stimuli.

Elevated expression of RhoA and RhoC, as well as the Rho effector proteins ROCK1 and ROCK2, are commonly observed in human cancers, including in the progression of testicular germ cell tumours, small breast carcinomas with metastatic ability, invasion and metastasis of bladder cancer, tumor progression in ovarian carcinoma.

Progression of tumors to invasive and metastatic forms requires that tumor cells undergo dramatic morphologic changes, a process regulated by Rho GTPases. Actomyosin contractility is a mechanism by which cells exert locomotory force against their environment. Signalling downstream of the small GTPase Rho increases contractility through ROCK-mediated regulation of myosin-II light chain (MLC2) phosphorylation.

The ROCK kinases are thought to participate in the induction of focal adhesions and stress fibers and to mediate calcium sensitization of smooth muscle contraction by enhancing phosphorylation of the regulatory light chain of myosin.

In vivo studies have also shown that ROCK inhibition reduced the invasiveness of several tumor cell lines. ROCK inhibitors, such as Y-27632 or WF-536, have been used in some studies to demonstrate these properties.

Inhibitors of ROCKs have been suggested for use in the treatments of a variety of diseases. These include cardiovascular diseases such as hypertension, chronic and congestive heart failure, cardiac hypertrophy, restenosis, chronic renal failure and atherosclerosis. Also, because of its muscle relaxing properties, inhibitors may also be suitable for asthma, male erectile dysfunction, female sexual dysfunction and over-active bladder I syndrome.

ROCK inhibitors have been shown to possess anti-inflammatory properties. Thus they can be used as treatment for neuroinflammatory diseases such as stroke, multiple sclerosis, Alzheimer's disease, Parkinson's disease, amyotrophic lateral sclerosis and inflammatory pain, as well as other inflammatory diseases such as rheumatoid arthritis, irritable bowel syndrome, and inflammatory bowel disease. Based on their neurite outgrowth inducing effects, ROCK inhibitors could be useful drugs for neuronal regeneration, inducing new axonal growth and axonal rewiring across lesions within the CNS. ROCK inhibitors are therefore likely to be useful for regenerative treatment of CNS disorders such as spinal cord injury, acute neuronal injury (stroke, traumatic brain injury), Parkinsons disease, Alzheimers disease and other neurodegenerative disorders. Since ROCK inhibitors reduce cell proliferation and cell migration, they could be useful in treating cancer and tumor metastasis. Finally, there is evidence to suggest that ROCK inhibitors suppress cytoskeletal rearrangement upon virus invasion, thus they also have potential therapeutic value in anti-viral and anti-bacterial applications. ROCK inhibitors are also useful for the treatment of insulin resistance and diabetes.

### ROCK Inhibitor Y-27632

Adhesion of tumour cells to host cell layers and subsequent transcellular migration are pivotal steps in cancer invasion and metastasis. The small GTPase Rho controls cell adhesion and motility through reorganization of the actin cytoskeleton and regulation of actomyosin contractility. Cultured rat MM1 hepatoma cells migrate in a serum-dependent, Rho-mediated manner, through a mesothelial cell monolayer in vitro. Among several proteins isolated as putative target molecules of Rho, the ROCK kinases are thought to participate in the induction of focal adhesions and stress fibres in cultured cells, and to mediate calcium sensitization of smooth muscle contraction by enhancing phosphorylation of the regulatory light chain of myosin. Transfection of MM1 cells with cDNA encoding a dominant active mutant of ROCK conferred invasive activity independently of serum and Rho. In contrast, expression of a dominant negative, kinase-defective ROCK mutant substantially attenuated the invasive phenotype.

A specific ROCK inhibitor (Y-27632) blocked both Rho-mediated activation of actomyosin and invasive activity of these cells. Furthermore, continuous delivery of this inhibitor using osmotic pumps considerably reduced the dissemination of MM1 cells implanted into the peritoneal cavity of syngeneic rats. These results indicate that ROCK plays an essential part in tumor cell invasion, and demonstrate its potential as a therapeutic target for the prevention of cancer invasion and metastasis.

VEGF induced the activation of RhoA and recruited RhoA to the cell membrane of human ECs. This increase in RhoA activity is necessary for the VEGF-induced reorganization of the F-actin cytoskeleton, as demonstrated by adenoviral transfection of dominant-negative RhoA. Rho kinase mediated this effect of RhoA, as was demonstrated by the use of Y-27632, a specific inhibitor of Rho kinase. Inhibition of Rho kinase prevented the VEGF-enhanced EC migration in response to mechanical wounding but had no effect on basal EC migration. Furthermore, in an in vitro model for angiogenesis, inhibition of either RhoA or Rho kinase attenuated the VEGF-mediated ingrowth of ECs in a 3-dimensional fibrin matrix. CONCLUSIONS: VEGF-induced cytoskeletal changes in ECs require RhoA and Rho kinase, and activation of RhoA/Rho kinase signaling is involved in the VEGF-induced in vitro EC migration and angiogenesis.

Y-27632 can relax smooth muscle and increase vascular blood flow. Y-27632 is a small molecule that can enter cells and is not toxic in rats after oral administration of 30 mg/kg for 10 days. Effective doses for the use of this compound are approximately 30 uM. It reduces blood pressure in hypertensive rats, but does not affect blood pressure in normal rats. This has led to the identification of Rho signalling antagonists in treatment of hypertension (Somlyo, 1997 Nature 389:908; Uehata et al., 1997 Nature 389:990).

The use of a specific inhibitor of ROCK, Y-27632 (Uehata, et al., Nature, 389, 990 994, 1997, Davies, et al., Biochemical Journal., 351, 95-105, 2000, and Ishizaki, et al., Molecular Pharmacology., 57, 976-983, 2000), has demonstrated a role for this enzyme in Ca2+ independent regulation of contraction in a number of tissues, including vascular (Uehata, et al., Nature., 389, 990-994, 1997), airway (Ilikuka et al., European Journal of 30 Pharmacology., 406, 273-279, 2000) and genital (Chitaley et al., Nature Medicine., 7(1), 119-122, 2001) smooth muscles. In addition, Jezior et al. British Journal of Pharmacology., 134, 78-87, 2001 have shown that Y-27632 attenuates bethanechol-evoked contractions in isolated rabbit urinary 35 bladder smooth muscle.

The Rho kinase inhibitor Y-27632 has been tested for the following disease applications:
Hypertension (Uehata et al., 1997 IBID; Chitaley et al., 2001a IBID; Chrissobolis and 15 Sobey, 2001 C. Circ. Res 88:774)
Asthma (lizuka et al., 2000 Eur. J. Pharmacol 406:273; Nakahara et al. Eur. J. Pharmacol 389:103, 2000)
Pulmonary vasoconstriction (Takamura et al., 2001 Hepatology 33:577)
Vascular disease (Miyata et al., 2000 Thromb Vasc Biol 20:2351; Robertson et al., 2000 Br. J. Pharmacol 131:5)
Penile erectile dysfunction (Chitaley et al., 2001b Nature Medicine 7:119; Mills et al., 2001 J. Appl. Physiol. 91: 1269; Rees et al., Br. J. Pharmacol 133:455 2001)
Glaucoma (Honjo et al., 2001 Methods Enzymol 42:137; Rao et al., 2001 Invest. Opthalmol. Urs. Sci. 42:1029)
Cell transformation (Sahai et al., 1999 Curr. Biol. 9:136-5)
Prostate cancer metastasis (Somlyo et al., 2000 BBRC 269:652)
Hepatocellular carcinoma and metastasis (Imamura et al., 2000; Takamura et al., 2001)
Liver fibrosis (Tada et al., 2001 J. Hepatol 34:529; Wang et al., 2001 Am. J. Respir. Cell Mol. Biol. 25:628)
Kidney fibrosis (Ohlci et al., J. Heart Lung Transplant 20:956 2001)
Cardioprotection and allograft survival (Ohlci et al., 2001 IBID)
Cerebral vasospasm (Sato et al., 2000 Circ. Res 87: 195).

### ROCK Kinase and Cardiovascular Disease

There is growing evidence that ROCKs, the immediate downstream targets of the small guanosine triphosphate-binding protein Rho, may contribute to cardiovascular disease. ROCKs play a central role in diverse cellular functions such as smooth muscle contraction, stress fiber formation and cell migration and proliferation. Overactivity of ROCKs is observed in cerebral ischemia, coronary vasospasm, hypertension, vascular inflammation, arteriosclerosis and atherosclerosis. ROCKs, therefore, may be an important and still relatively unexplored therapeutic target in cardiovascular disease. Recent experimental and clinical studies using ROCK inhibitors such as Y-27632 and fasudil have revealed a critical role of ROCKs in embryonic development, inflammation and oncogenesis. This review will focus on the potential role of ROCKs in cellular functions and discuss the prospects of ROCK inhibitors as emerging therapy for cardiovascular diseases.

Abnormal smooth-muscle contractility may be a major cause of disease states such as hypertension, and a smooth-muscle relaxant that modulates this process would be useful therapeutically. Smooth-muscle contraction is regulated by the cytosolic Ca2+ concentration and by the Ca2+ sensitivity of myofilaments: the former activates myosin light-chain kinase and the latter is achieved partly by inhibition of myosin phosphatase.

Rho signaling pathways in vascular smooth muscle cells are highly activated in hypertension, a condition associated with a variety of vascular diseases, including restenosis injury and atherosclerosis.

Hypertension is a cardiovascular disorder characterized by increased peripheral vascular resistance and/or vascular structural remodeling. Recently, rapidly growing evidence from hypertensive animal models suggests that small GTPase Rho and its downstream effector, Rho-kinase, play an important role in the pathogenesis of hypertension. Activation of the Rho/Rho-kinase pathway is essential for smooth muscle contractility in hypertension. A greater RhoA expression and an enhanced RhoA activity have been observed in aortas of hypertensive rats, such as genetic spontaneously hypertensive rats and N(omega)-nitro-L-arginine methyl ester-induced hypertension.

### ROCK Kinase and Neurological Diseases

Abnormal activation of the Rho/ROCK pathway has been observed in various disorders of the central nervous system. Injury to the adult vertebrate brain and spinal cord activates ROCKs, thereby inhibiting neurite growth and sprouting. Inhibition of ROCKs results in accelerated regeneration and enhanced functional recovery after spinal-cord injury in mammals, and inhibition of the Rho/ROCK pathway has also proved to be efficacious in animal models of stroke, inflammatory and demyelinating diseases, Alzheimer's disease and neuropathic pain. ROCK inhibitors therefore have potential for preventing neurodegeneration and stimulating neuroregeneration in various neurological disorders.

The development of a neuron requires a series of steps that begins with migration from its birth place and initiation of process outgrowth, and ultimately leads to differentiation and the formation of connections that allow it to communicate with appropriate targets. Over the past several years, it has become clear that the Rho family of GTPases and related molecules play an important role in various aspects of neuronal development, including neurite outgrowth and differentiation, axon pathfinding, and dendritic spine formation and maintenance.

One common denominator for both neurite outgrowth inhibition and neurite repulsion is actin rearrangements within the growth cone. Central to the regulation of the actin cytoskeleton in both neuronal and non-neuronal cells is the Rho family of small GTPases. Rho family members cycle between an inactive GDP-bound form and an active GTP-bound form. Several lines of evidence suggest that manipulating the activity state of Rho GTPases may modulate growth cone collapse and neurite outgrowth inhibition.

More recently, behaviorally, inactivation of Rho pathway can induce rapid recovery of locomotion and progressive recuperation of forelimb-hindlimb coordination. These findings provide evidence that the Rho signaling pathway is a potential target for therapeutic interventions after spinal cord injury.

WO 93/13072 (Italfarmaco) discloses a class of bis-sulphonamido diamines as protein kinase inhibitors.

Purines and purine analogues and derivatives have been disclosed as having a wide range of different biological activities.

For example, WO03/057696 (Eisai) discloses a class of indolyl-deazapurines for treating inflammatory or autoimmune or proliferative diseases.

WO 99/65909 (Pfizer) discloses a class of pyrrole[2,3-d pyrimidine compounds as inhibitors of protein tyrosine kinases such as Janus kinase 3. The compounds are described as having a range of therapeutic uses.

Semonsky et al. Czech. Chem. Comm. (1960), 25, 1091-1099, disclose derivatives of 6-carboxyalkylthiopurine as anti-cancer agents.

Noell et al., J. Org. Chem., (1958), 23, 1547-1550 disclose 4-(substituted amino)pyrazole[3,4-d]pyrimidines as potential anti-tumour agents.

Lettre et al., Naturwissenschaften (1958), 45, 364 disclose several aminoalkyl-aminopurine derivatives having activity against tumour cells.

US 2003/0139427 (OSI) discloses pyrrolidine- and piperidine-substituted purines and purine analogues having adenosine receptor binding activity.

WO 2004/043380 (Harvard College et al.) discloses technetium and rhenium labelled imaging agents containing disubstituted piperidine metal ion-chelating ligands.

WO 97/38665 (Merck) discloses gem-disubstituted piperidine derivatives having farnesyl transferase inhibitory activity.

EP 1568699 (Eisai) discloses 1,3-dihydroimidazole fused ring compounds having DPPIV-inhibiting activity. The compounds are described as having a range of potential uses including the treatment of cancer.

US 2003/0073708 and US 2003/045536 (both in the name of Castelhano et al), WO 02/057267 (OSI Pharmaceuticals) and WO 99/62518 (Cadus Pharmaceutical Corporation) each disclose a class of 4-aminodeazapurines in which the 4-amino group can form part of a cyclic amine such as azetidine, pyrrolidine and piperidine. The compounds are described as having adenosine receptor antagonist activity.

U.S. Pat. No. 6,162,804 (Merck) discloses a class of benzimidazoles and imidazopyridines as tyrosine kinase inhibitors.

### SUMMARY OF THE INVENTION

The present invention is based, at least in part, on the discovery of a variety of novel medical applications for compounds having the formula (I) as defined herein.

In particular, the present inventors have now discovered that compounds of the formula (I) find application in: (a) the treatment or prophylaxis of a disease or condition in which the modulation (e.g. inhibition) of ROCK kinase or protein kinase p70S6K is indicated; and/or (b) the treatment of a subject or patient population in which the modulation (e.g. inhibition) of ROCK kinase or protein kinase p70S6K is indicated.

Accordingly, in a first aspect, the invention provides a compound of the formula (I):

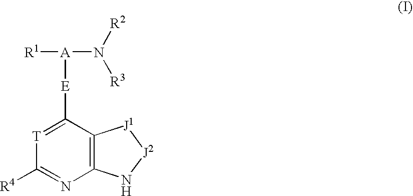

or salts, solvates, tautomers or N-oxides thereof, wherein

T is N or a group CR⁵;

J¹-J² represents a group selected from N═C(R⁶), (R⁷)C═N, (R⁸)N—C(O), (R⁸)₂C—C(O), N═N and (R⁷)C═C(R⁶);

A is a saturated hydrocarbon linker group containing from 1 to 7 carbon atoms, the linker group having a maximum chain length of 5 atoms extending between R¹ and NR²R³ and a maximum chain length of 4 atoms extending between E and NR²R³, wherein one of the carbon atoms in the linker group may optionally be replaced by an oxygen or nitrogen atom; and wherein the carbon atoms of the linker group A may optionally bear one or more substituents selected from oxo, fluorine and hydroxy, provided that the hydroxy group when present is not located at a carbon atom α with respect to the NR²R³ group and provided that the oxo group when present is located at a carbon atom α with respect to the NR²R³ group;

E is a monocyclic or bicyclic carbocyclic or heterocyclic group or an acyclic group X-G wherein X is selected from CH₂, O, S and NH and G is a C₁₋₄ alkylene chain wherein one of the carbon atoms is optionally replaced by O, S or NH;

R¹ is hydrogen or an aryl or heteroaryl group;

R² and R³ are independently selected from hydrogen, C₁₋₄ hydrocarbyl and C₁₋₄ acyl wherein the hydrocarbyl and acyl groups are optionally substituted by one or more substituents selected from fluorine, hydroxy, amino, methylamino, dimethylamino, methoxy and a monocyclic or bicyclic aryl or heteroaryl group;

or R² and R³ together with the nitrogen atom to which they are attached form a cyclic group selected from an imidazole group and a saturated monocyclic heterocyclic group having 4-7 ring members and optionally containing a second heteroatom ring member selected from O and N;

or one of R² and R³ together with the nitrogen atom to which they are attached and one or more atoms from the linker group A form a saturated monocyclic heterocyclic group having 4-7 ring members and optionally containing a second heteroatom ring member selected from O and N. the monocyclic heterocyclic group being optionally substituted by one or more C₁₋₄ alkyl groups;

or NR²R³ and the carbon atom of linker group A to which it is attached together form a cyano group; or

R¹, A and NR²R³ together form a cyano group; and

R⁴, R⁵, R⁶, R⁷ and R⁸ are each independently selected from hydrogen; halogen; C₁₋₆ hydrocarbyl optionally substituted by halogen, hydroxy or C₁₋₂ alkoxy; cyano; CONH₂; CONHR⁹; CF₃; NH₂; NHCOR⁹ and NHCONHR⁹;

R⁹ is phenyl or benzyl each optionally substituted by one or substituents selected from halogen, hydroxy, trifluoromethyl, cyano, nitro, carboxy, amino, mono- or di-C₁₋₄ hydrocarbylamino; a group R^{a}-R^{b} wherein R^{a} is a bond, O, CO, X¹C(X²), C(X²)X¹, X¹C(X²)X¹, S, SO, SO₂, NR^{c}, SO₂NR^{c} or NR^{c}SO₂; and R^{b} is selected from hydrogen, heterocyclic groups having from 3 to 12 ring members, and a C₁₋₈ hydrocarbyl group optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹;

R^{c} is selected from hydrogen and C₁₋₄ hydrocarbyl; and

X¹ is O, S or NR^{c} and X² is ═O, ═S or ═NR^{c};

wherein the compound is for use in: (a) the treatment or prophylaxis of a disease or condition in which the modulation (e.g. inhibition) of ROCK kinase or protein kinase p70S6K is indicated; and/or (b) the treatment of a subject or patient population in which the modulation (e.g. inhibition) of ROCK kinase or protein kinase p70S6K is indicated.

In another aspect, the invention provides a compound of the formula (Ia):

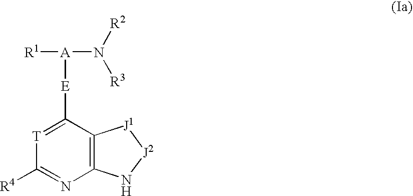

or salts, solvates, tautomers or N-oxides thereof, wherein

T is N or a group CR⁵;

J¹-J² represents a group selected from N═C(R⁶), (R⁷)C═N, (R⁸)N—C(O), (R⁸)₂C—C(O), N═N and (R⁷)C═C(R⁶);

A is a saturated hydrocarbon linker group containing from 1 to 7 carbon atoms, the linker group having a maximum chain length of 5 atoms extending between R¹ and NR²R³ and a maximum chain length of 4 atoms extending between E and NR²R³, wherein one of the carbon atoms in the linker group may optionally be replaced by an oxygen or nitrogen atom; and wherein the carbon atoms of the linker group A may optionally bear one or more substituents selected from oxo, fluorine and hydroxy, provided that the hydroxy group when present is not located at a carbon atom α with respect to the NR²R³ group and provided that the oxo group when present is located at a carbon atom α with respect to the NR²R³ group;

E is a monocyclic or bicyclic carbocyclic or heterocyclic group or an acyclic group X-G wherein X is selected from CH₂, O S and NH and G is a C₁₋₄ alkylene chain wherein one of the carbon atoms is optionally replaced by O, S or NH;

R¹ is hydrogen or an aryl or heteroaryl group;

R² and R³ are independently selected from hydrogen, C₁₋₄ hydrocarbyl and C₁₋₄ acyl;

or R² and R³ together with the nitrogen atom to which they are attached form a saturated monocyclic heterocyclic group having 4-7 ring members and optionally containing a second heteroatom ring member selected from O and N. the monocyclic heterocyclic group being optionally substituted by one or more C₁₋₄ alkyl groups;

or one of R² and R³ together with the nitrogen atom to which they are attached and one or more atoms from the linker group A form a saturated monocyclic heterocyclic group having 4-7 ring members and optionally containing a second heteroatom ring member selected from O and N. the monocyclic heterocyclic group being optionally substituted by one or more C₁₋₄ alkyl groups;

or NR²R³ and the carbon atom of linker group A to which it is attached together form a cyano group; or

R¹, A and NR²R³ together form a cyano group; and

R⁴, R⁵, R⁶, R⁷ and R⁸ are each independently selected from hydrogen; halogen; C₁₋₆ hydrocarbyl optionally substituted by halogen, hydroxy or C₁₋₂ alkoxy; cyano; CONH₂; CONHR⁹; CF₃; NH₂; NHCOR⁹ and NHCONHR⁹;

R⁹ is phenyl or benzyl each optionally substituted by one or substituents selected from halogen, hydroxy, trifluoromethyl, cyano, nitro, carboxy, amino, mono- or di-C₁₋₄ hydrocarbylamino; a group R^{a}-R^{b} wherein R^{a} is a bond, 0 CO, X¹C(X²), C(X²)X¹, X¹C(X²)X¹, S, SO, SO₂, NR^{c}, SO₂NR^{c} or NR^{c}SO₂; and R^{b} is selected from hydrogen, heterocyclic groups having from 3 to 12 ring members, and a C₁₋₈ hydrocarbyl group optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹;

R^{c} is selected from hydrogen and C₁₋₄ hydrocarbyl; and

X¹ is O, S or NR^{c} and X² is ═O, ═S or ═NR^{c};

wherein the compound is for use in: (a) the treatment or prophylaxis of a disease or condition in which the modulation (e.g. inhibition) of ROCK kinase or protein kinase p70S6K is indicated; and/or (b) the treatment of a subject or patient population in which the modulation (e.g. inhibition) of ROCK kinase or protein kinase p70S6K is indicated.
In a further aspect, the invention provides a compound of the formula (Ib):

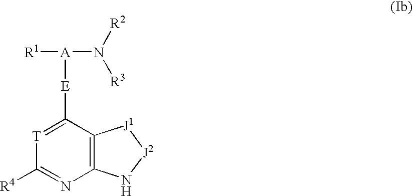

or salts, solvates, tautomers or N-oxides thereof, wherein

T is N or a group CR⁵;

J¹-J² represents a group selected from N═C(R⁶), (R⁷)C═N, (R⁸)N—C(O), (R⁸)₂C—C(O), N═N and (R⁷)C═C(R⁶);

A is a saturated hydrocarbon linker group containing from 1 to 7 carbon atoms, the linker group having a maximum chain length of 5 atoms extending between R¹ and NR²R³ and a maximum chain length of 4 atoms extending between E and NR²R³, wherein one of the carbon atoms in the linker group may optionally be replaced by an oxygen or nitrogen atom; and wherein the carbon atoms of the linker group A may optionally bear one or more substituents selected from oxo, fluorine and hydroxy, provided that the hydroxy group when present is not located at a carbon atom α with respect to the NR²R³ group and provided that the oxo group when present is located at a carbon atom α with respect to the NR²R³ group;

E is a monocyclic or bicyclic carbocyclic or heterocyclic group or an acyclic group X-G wherein X is selected from CH₂, O S and NH and G is a C₁₋₄ alkylene chain wherein one of the carbon atoms is optionally replaced by O, S or NH;

R¹ is hydrogen or an aryl or heteroaryl group;

R² and R³ are independently selected from hydrogen, C₁₋₄ hydrocarbyl and C₁₋₄ acyl wherein the hydrocarbyl and acyl groups are optionally substituted by one or more substituents selected from fluorine, hydroxy, amino, methylamino, dimethylamino, methoxy and a monocyclic or bicyclic aryl or heteroaryl group;

or R² and R³ together with the nitrogen atom to which they are attached form a cyclic group selected from an imidazole group and a saturated monocyclic heterocyclic group having 4-7 ring members and optionally containing a second heteroatom ring member selected from O and N;

or one of R² and R³ together with the nitrogen atom to which they are attached and one or more atoms from the linker group A form a saturated monocyclic heterocyclic group having 4-7 ring members and optionally containing a second heteroatom ring member selected from O and N. the monocyclic heterocyclic group being optionally substituted by one or more C₁₋₄ alkyl groups;

or NR²R³ and the carbon atom of linker group A to which it is attached together form a cyano group; or

R¹, A and NR²R³ together form a cyano group; and

R⁴, R⁵, R⁶, R⁷ and R⁸ are each independently selected from hydrogen; halogen; C₁₋₆ hydrocarbyl optionally substituted by halogen, hydroxy or C₁₋₂ alkoxy; cyano; CONH₂; CONHR⁹; CF₃; NH₂; NHCOR⁹ and NHCONHR⁹;

R⁹ is phenyl or benzyl each optionally substituted by one or substituents selected from halogen, hydroxy, trifluoromethyl, cyano, nitro, carboxy, amino, mono- or di-C₁₋₄ hydrocarbylamino; a group R^{a}-R^{b} wherein R^{a} is a bond, O, CO, X¹C(X²), C(X²)X¹, X¹C(X²)X¹, S, SO, SO₂, NR^{c}, SO₂NR^{c} or NR^{c}SO₂; and R^{b} is selected from hydrogen, heterocyclic groups having from 3 to 12 ring members, and a C₁₋₈ hydrocarbyl group optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹;

R^{c} is selected from hydrogen and C₁₋₄ hydrocarbyl; and

X¹ is O, S or NR^{c} and X² is ═O, ═S or ═NR^{c};

provided that:
(a-i) when J¹-J² is (R⁷)C═C(R⁶) and E is a monocyclic or bicyclic group linked through a nitrogen atom to the ring containing T. then A contains no oxo substituent;
(a-ii) E is other than an unsubstituted or substituted indole group;
(a-iii) when J¹-J² is N═CH, then E-A(R¹)—NR²R³ is other than a group —S—(CH₂)₃—CONH₂ or —S—(CH₂)₃—CN;
(a-iv) when J¹-J² is CH═N, then E-A(R¹)—NR²R³ is other than a group —NH—(CH₂)ₙ N(CH₂CH₃)₂ where n is 2 or 3; and
(a-v) when J¹-J² is N═CH, then E-A(R¹)—NR²R³ is other than a group —NH—(CH₂)₂—NH₂ or —NH—(CH₂)₂—N(CH₃)₂;
wherein the compound is for use in: (a) the treatment or prophylaxis of a disease or condition in which the modulation (e.g. inhibition) of ROCK kinase or protein kinase p70S6K is indicated; and/or (b) the treatment of a subject or patient population in which the modulation (e.g. inhibition) of ROCK kinase or protein kinase p70S6K is indicated.
In another aspect, the invention provides a compound of the formula (Ic):

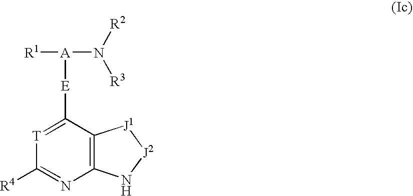

or salts, solvates, tautomers or N-oxides thereof, wherein

T is N or a group CR⁵;

J¹-J² represents a group selected from N═C(R⁶), (R⁷)C═N, (R⁸)N—C(O), (R⁸)₂C—C(O), N═N and (R⁷)C═C(R⁶);

A is a saturated hydrocarbon linker group containing from 1 to 7 carbon atoms, the linker group having a maximum chain length of 5 atoms extending between R¹ and NR²R³ and a maximum chain length of 4 atoms extending between E and NR²R³, wherein one of the carbon atoms in the linker group may optionally be replaced by an oxygen or nitrogen atom; and wherein the carbon atoms of the linker group A may optionally bear one or more substituents selected from fluorine and hydroxy, provided that the hydroxy group when present is not located at a carbon atom α with respect to the NR²R³ group;

E is a monocyclic carbocyclic or heterocyclic group;

R¹ is an aryl or heteroaryl group;

R² and R³ are independently selected from hydrogen, C₁₋₄ hydrocarbyl and C₁₋₄ acyl wherein the hydrocarbyl and acyl groups are optionally substituted by one or more substituents selected from fluorine, hydroxy, amino, methylamino, dimethylamino, methoxy and a monocyclic or bicyclic aryl or heteroaryl group;

or R² and R³ together with the nitrogen atom to which they are attached form a saturated monocyclic heterocyclic group having 4-7 ring members and optionally containing a second heteroatom ring member selected from O and N;

or one of R² and R³ together with the nitrogen atom to which they are attached and one or more atoms from the linker group A form a saturated monocyclic heterocyclic group having 4-7 ring members and optionally containing a second heteroatom ring member selected from O and N, the monocyclic heterocyclic group being optionally substituted by one or more C₁₋₄ alkyl groups;

or NR²R³ and the carbon atom of linker group A to which it is attached together form a cyano group; or

R¹, A and NR²R³ together form a cyano group; and

R⁴, R⁵, R⁶, R⁷ and R⁸ are each independently selected from hydrogen; halogen; C₁₋₆ hydrocarbyl optionally substituted by halogen, hydroxy or C₁₋₂ alkoxy; cyano; CONH₂; CONHR⁹; CF₃; NH₂; NHCOR⁹ and NHCONHR⁹;

R⁹ is phenyl or benzyl each optionally substituted by one or substituents selected from halogen, hydroxy, trifluoromethyl, cyano, nitro, carboxy, amino, mono- or di-C₁₋₄ hydrocarbylamino; a group R^{a}-R^{b} wherein R^{a} is a bond, O, CO, X¹C(X²), C(X²)X¹, X¹C(X²)X¹, S, SO, SO₂, NR^{c}, SO₂NR^{c} or NR^{c}SO₂; and R^{b} is selected from hydrogen, heterocyclic groups having from 3 to 12 ring members, and a C₁₋₈ hydrocarbyl group optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹;

R^{c} is selected from hydrogen and C₁₋₄ hydrocarbyl; and

X¹ is O, S or NR^{c} and X² is ═O, ═S or ═NR^{c};

wherein the compound is for use in: (a) the treatment or prophylaxis of a disease or condition in which the modulation (e.g. inhibition) of ROCK kinase or protein kinase p70S6K is indicated; and/or (b) the treatment of a subject or patient population in which the modulation (e.g. inhibition) of ROCK kinase or protein kinase p70S6K is indicated.
In further aspects, the invention provides:
A compound per se of the formula (I) as defined herein wherein the compound is for use in: (a) the treatment or prophylaxis of a disease or condition in which the modulation (e.g. inhibition) of ROCK kinase or protein kinase p70S6K is indicated; and/or (b) the treatment of a subject or patient population in which the modulation (e.g. inhibition) of ROCK kinase or protein kinase p70S6K is indicated.
A compound of the formula (I) as defined herein wherein the compound is for use in: (a) the treatment or prophylaxis of a disease or condition in which the modulation (e.g. inhibition) of ROCK kinase or protein kinase p70S6K is indicated; and/or (b) the treatment of a subject or patient population in which the modulation (e.g. inhibition) of ROCK kinase or protein kinase p70S6K is indicated.
The use of a compound of the formula (I) as defined herein for the manufacture of a medicament for: (a) the treatment or prophylaxis of a disease or condition in which the modulation (e.g. inhibition) of ROCK kinase or protein kinase p70S6K is indicated; and/or (b) the treatment of a subject or patient population in which the modulation (e.g. inhibition) of ROCK kinase or protein kinase p70S6K is indicated.
A method for the prophylaxis or treatment of a disease state or condition mediated by ROCK kinase or protein kinase p70S6K, which method comprises administering to a subject in need thereof a compound of the formula (I) as defined herein.
A method for treating a disease or condition comprising or arising from abnormal cell growth or abnormally arrested cell death in a mammal, the method comprising administering to the mammal a compound of the formula (I) as defined herein in an amount effective to inhibit ROCK kinase or protein kinase p70S6K activity.
A method of inhibiting ROCK kinase or protein kinase p70S6K, which method comprises contacting the kinase with a kinase-inhibiting compound of the formula (I) as defined herein.
A method of modulating a cellular process (for example cell division) by inhibiting the activity of ROCK kinase or protein kinase p70S6K using a compound of the formula (I) as defined herein.
A compound of the formula (I) as defined herein for use in the prophylaxis or treatment of a disease state or condition mediated by ROCK kinase or protein kinase p70S6K.
The use of a compound of the formula (I) as defined herein for the manufacture of a medicament for the prophylaxis or treatment of a disease state or condition mediated by ROCK kinase or protein kinase p70S6K.
The use of a compound of the formula (I) as defined herein for the manufacture of a medicament for the prophylaxis or treatment of a disease state or condition arising from abnormal cell growth or abnormally arrested cell death mediated by ROCK kinase or protein kinase p70S6K.
A method for alleviating or reducing the incidence of a disease or condition comprising or arising from abnormal cell growth or abnormally arrested cell death in a mammal mediated by ROCK kinase or protein kinase p70S6K, which method comprises administering to the mammal a compound of the formula (I) as defined herein in an amount effective in inhibiting abnormal cell growth.
The use of a compound of the formula (I) as defined herein for the manufacture of a medicament for the prophylaxis or treatment of any one of the disease states or conditions disclosed herein.
A method for the treatment or prophylaxis of any one of the disease states or conditions disclosed herein, which method comprises administering to a patient (e.g. a patient in need thereof) a compound (e.g. a therapeutically effective amount) of the formula (I) as defined herein.
A method for alleviating or reducing the incidence of a disease state or condition disclosed herein, which method comprises administering to a patient (e.g. a patient in need thereof) a compound (e.g. a therapeutically effective amount) of the formula (I) as defined herein.
A method for the diagnosis and treatment of a disease state or condition mediated by ROCK kinase or protein kinase p70S6K, which method comprises (i) screening a patient to determine whether a disease or condition from which the patient is or may be suffering is one which would be susceptible to treatment with a compound having activity against ROCK kinase or protein kinase p70S6K; and (ii) where it is indicated that the disease or condition from which the patient is thus susceptible, thereafter administering to the patient a compound of the formula (I) as defined herein.
The use of a compound of the formula (I) as defined herein for the manufacture of a medicament for the treatment or prophylaxis of a disease state or condition in a patient who has been screened and has been determined as suffering from, or being at risk of suffering from, a disease or condition which would be susceptible to treatment with a compound having activity against ROCK kinase or protein kinase p70S6K.

### General Preferences and Definitions

As used herein, the terms “ROCK kinase(s)” and “ROCK(s)” are synonomous generic terms embracing all members of the ROCK kinase family, so including both ROCK1 and ROCK2 as species within the genus. References inter alia to ROCK kinase inhibitors, ROCK kinase modulation and ROCK kinase activity are to be interpreted accordingly.

The term “Rho protein” is a term of art used to define a large family of GTP-binding proteins that are involved in regulation of actin organization, including RhoA and RhoC.

As used herein, the term “Rho signalling pathway” defines any cellular signaling pathway in which one or more members of the Rho proteins are involved. Particularly relevant to the invention are Rho signaling pathways in which a ROCK kinase (e.g. ROCK1 and/or ROCK2) is a proximate effector (e.g. a binding partner) for one or more Rho protein(s), and such Rho signaling pathways are preferred in embodiments of the invention defined inter alia by reference to a Rho signaling pathway.

As used herein, the term “modulation”, as applied to the ROCK kinase or protein kinase p70S6K as described herein, is intended to define a change in the level of biological activity of the kinases. Thus, modulation encompasses physiological changes which effect an increase or decrease in kinase activity. In the latter case, the modulation may be described as “inhibition”. The modulation may arise directly or indirectly, and may be mediated by any mechanism and at any physiological level, including for example at the level of gene expression (including for example transcription, translation and/or post-translational modification), at the level of expression of genes encoding regulatory elements which act directly or indirectly on the levels of kinase activity, or at the level of enzyme (e.g. ROCK or p70S6K) activity (for example by allosteric mechanisms, competitive inhibition, active-site inactivation, perturbation of feedback inhibitory pathways etc.). Thus, modulation may imply elevated/suppressed expression or over- or under-expression of the kinase, including gene amplification (i.e. multiple gene copies) and/or increased or decreased expression by a transcriptional effect, as well as hyper-(or hypo-)activity and (de)activation of the kinase (including (de)activation) by mutation(s). The terms “modulated” and “modulate” are to be interpreted accordingly.

As used herein, the term “mediated”, as used in conjunction with the kinases (i.e. the ROCKs and protein kinase p70S6K) as described herein (and applied for example to various physiological processes, diseases, states, conditions, therapies, treatments or interventions) is intended to operate limitatively so that the various processes, diseases, states, conditions, treatments and interventions to which the term is applied are those in which the kinase plays a biological role. In cases where the term is applied to a disease, state or condition, the role played by the kinase may be direct or indirect and may be necessary and/or sufficient for the manifestation of the symptoms of the disease, state or condition (or its aetiology or progression). Thus, kinase activity (and in particular aberrant levels of kinase activity, e.g. kinase over-expression) need not necessarily be the proximal cause of the disease, state or condition: rather, it is contemplated that ROCK- or protein kinase p70S6K-mediated diseases, states or conditions include those having multifactorial aetiologies and complex progressions in which the kinase is only partially involved. In cases where the term is applied to treatment, prophylaxis or intervention (e.g. in the “ROCK-mediated treatments”, “ROCK-mediated prophylaxis”, “protein kinase p70S6K-mediated treatments” and “p70S6K-mediated prophylaxis” of the invention), the role played by the kinase may be direct or indirect and may be necessary and/or sufficient for the operation of the treatment, prophylaxis or outcome of the intervention. Many ROCK-mediated physiological processes, diseases, states, conditions, therapies, treatments or interventions of the invention involve the Rho signaling pathway (as herein defined) and may therefore, by extension, be dubbed “Rho-mediated” physiological processes, diseases, states, conditions, therapies, treatments or interventions.

The term “indicated” is a term of art used herein in relation to a disease, condition, subject or patient population to convey the clinical desirability or necessity of a particular intervention in relation to that disease, condition, subject or patient population. Thus, references herein to a disease, condition, subject or patient population “in which the modulation (e.g. inhibition) of ROCK kinase or protein kinase p70S6K is indicated” is intended to define those diseases etc. in which modulation of ROCK kinase or protein kinase p70S6K is either clinically desirable or necessary. This might be the case, for example, where modulation of ROCK kinase or protein kinase p70S6K would be palliative, preventative or (at least partially) curative.

The term “intervention” is a term of art used herein to define any agency which effects a physiological change at any level. Thus, the intervention may comprises the induction or repression of any physiological process, event, biochemical pathway or cellular/biochemical event. The interventions of the invention typically effect (or contribute to) the therapy, treatment or prophylaxis of a disease or condition.

Where they do not already apply, any one or more of the following optional provisos may apply in any combination to any one or more of formulae (I), (Ia), (Ib), (Ic), (II), (IIa), (IIb), (III) or any sub-group or embodiment thereof as defined herein, and for any one or more of the aspects of the invention set out above and elsewhere herein.

(a-i) When J¹-J² is (R⁷)C═C(R⁶) and E is a monocyclic or bicyclic group linked through a nitrogen atom to the ring containing T. then A contains no oxo substituent.
(a-ii) E is other than an unsubstituted or substituted indole group;
(a-iii) when J¹-J² is N═CH, then E-A(R¹)—NR²R³ is other than a group —S—(CH₂)₃—CONH₂ or —S—(CH₂)₃—CN.
(a-iv) When J¹-J² is CH═N, then E-A(R¹)—NR²R³ is other than a group —NH—(CH₂)ₙ N(CH₂CH₃)₂ where n is 2 or 3.
(a-v) When J¹-J² is N═CH, then E-A(R¹)—NR²R³ is other than a group —NH—(CH₂)₂—NH₂ or —NH—(CH₂)₂—N(CH₃)₂.
(b-i) E may be other than an unsubstituted or substituted indole group wherein A is attached to the benzene ring of the indole group.
(b-ii) When E is a monocyclic or bicyclic group linked through a nitrogen atom to the ring containing T. and one of R² and R³ together with the nitrogen atom to which they are attached and one or more atoms from A form a saturated monocyclic heterocyclic group optionally containing a second heteroatom ring member, then J¹-J² may be other than (R⁷)C═C(R⁶).
(b-iii) The moiety E-A(R¹)—NR²R³ may be other than an aminoalkylamino or alkylaminoalkylamino group.
(b-iv) When R¹ is hydrogen, E may be other than an acyclic group X-G.
(b-v) When E is piperidine or pyrrolidine, the moiety A(R¹)—NR²R³ may be other than pyrrolidinylethyl or pyrrolidinylmethyl.

The following general preferences and definitions shall apply to each of the moieties A, E, J¹, J², T and R¹ to R⁹ and any sub-definition, sub-group or embodiment thereof, unless the context indicates otherwise.

Any references to Formula (I) herein shall be taken also to refer to formulae (Ia), (Ib), (Ic), (II), (IIa), (IIb), (III) and any other sub-group of compounds within formula (I), or embodiment thereof, unless the context requires otherwise.

In this specification, references to “the bicyclic group”, when used in regard to the point of attachment of the group E shall, unless the context indicates otherwise, be taken to refer to the group:

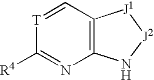

References to “carbocyclic” and “heterocyclic” groups as used herein shall, unless the context indicates otherwise, include both aromatic and non-aromatic ring systems. In general, such groups may be monocyclic or bicyclic and may contain, for example, 3 to 12 ring members, more usually 5 to 10 ring members. Examples of monocyclic groups are groups containing 3, 4, 5, 6, 7, and 8 ring members, more usually 3 to 7, and preferably 5 or 6 ring members. Examples of bicyclic groups are those containing 8, 9, 10, 11 and 12 ring members, and more usually 9 or 10 ring members.

The carbocyclic or heterocyclic groups can be aryl or heteroaryl groups having from 5 to 12 ring members, more usually from 5 to 10 ring members. The term “aryl” as used herein refers to a carbocyclic group having aromatic character and the term “heteroaryl” is used herein to denote a heterocyclic group having aromatic character. The terms “aryl” and “heteroaryl” embrace polycyclic (e.g. bicyclic) ring systems wherein one or more rings are non-aromatic, provided that at least one ring is aromatic. In such polycyclic systems, the group may be attached by the aromatic ring, or by a non-aromatic ring. The aryl or heteroaryl groups can be monocyclic or bicyclic groups and can be unsubstituted or substituted with one or more substituents, for example one or more groups R¹⁰ as defined herein.

The term non-aromatic group embraces unsaturated ring systems without aromatic character, partially saturated and fully saturated carbocyclic and heterocyclic ring systems. The terms “unsaturated” and “partially saturated” refer to rings wherein the ring structure(s) contains atoms sharing more than one valence bond i.e. the ring contains at least one multiple bond e.g. a C═C, C≡C or N═C bond. The term “fully saturated” refers to rings where there are no multiple bonds between ring atoms. Saturated carbocyclic groups include cycloalkyl groups as defined below. Partially saturated carbocyclic groups include cycloalkenyl groups as defined below, for example cyclopentenyl, cycloheptenyl and cyclooctenyl.

Examples of heteroaryl groups are monocyclic and bicyclic groups containing from five to twelve ring members, and more usually from five to ten ring members. The heteroaryl group can be, for example, a five membered or six membered monocyclic ring or a bicyclic structure formed from fused five and six membered rings or two fused six membered rings. Each ring may contain up to about four heteroatoms typically selected from nitrogen, sulphur and oxygen. Typically the heteroaryl ring will contain up to 3 heteroatoms, more usually up to 2, for example a single heteroatom. In one embodiment, the heteroaryl ring contains at least one ring nitrogen atom. The nitrogen atoms in the heteroaryl rings can be basic, as in the case of an imidazole or pyridine, or essentially non-basic as in the case of an indole or pyrrole nitrogen. In general the number of basic nitrogen atoms present in the heteroaryl group, including any amino group substituents of the ring, will be less than five.

Examples of five membered heteroaryl groups include but are not limited to pyrrole, furan, thiophene, imidazole, furazan, oxazole, oxadiazole, oxatriazole, isoxazole, thiazole, isothiazole, pyrazole, triazole and tetrazole groups.

Examples of six membered heteroaryl groups include but are not limited to pyridine, pyrazine, pyridazine, pyrimidine and triazine.

A bicyclic heteroaryl group may be, for example, a group selected from:
a) a benzene ring fused to a 5- or 6-membered ring containing 1, 2 or 3 ring heteroatoms;
b) a pyridine ring fused to a 5- or 6-membered ring containing 1, 2 or 3 ring heteroatoms;
c) a pyrimidine ring fused to a 5- or 6-membered ring containing 1 or 2 ring heteroatoms;
d) a pyrrole ring fused to a a 5- or 6-membered ring containing 1, 2 or 3 ring heteroatoms;
e) a pyrazole ring fused to a a 5- or 6-membered ring containing 1 or 2 ring heteroatoms;
f) a pyrazine ring fused to a 5- or 6-membered ring containing 1 or 2 ring heteroatoms;
g) an imidazole ring fused to a 5- or 6-membered ring containing 1 or 2 ring heteroatoms;
h) an oxazole ring fused to a 5- or 6-membered ring containing 1 or 2 ring heteroatoms;
i) an isoxazole ring fused to a 5- or 6-membered ring containing 1 or 2 ring heteroatoms;
j) a thiazole ring fused to a 5- or 6-membered ring containing 1 or 2 ring heteroatoms;
k) an isothiazole ring fused to a 5- or 6-membered ring containing 1 or 2 ring heteroatoms;
l) a thiophene ring fused to a 5- or 6-membered ring containing 1, 2 or 3 ring heteroatoms;
m) a furan ring fused to a 5- or 6-membered ring containing 1, 2 or 3 ring heteroatoms;
n) a cyclohexyl ring fused to a 5- or 6-membered ring containing 1, 2 or 3 ring heteroatoms; and
o) a cyclopentyl ring fused to a 5- or 6-membered ring containing 1, 2 or 3 ring heteroatoms.

Particular examples of bicyclic heteroaryl groups containing a six membered ring fused to a five membered ring include but are not limited to benzofuran, benzthiophene, benzimidazole, benzoxazole, benzisoxazole, benzthiazole, benzisothiazole, isobenzofuran, indole, isoindole, indolizine, indoline, isoindoline, purine (e.g., adenine, guanine), indazole, benzodioxole and pyrazolopyridine groups.

Particular examples of bicyclic heteroaryl groups containing two fused six membered rings include but are not limited to quinoline, isoquinoline, chroman, thiochroman, chromene, isochromene, chroman, isochroman, benzodioxan, quinolizine, benzoxazine, benzodiazine, pyridopyridine, quinoxaline, quinazoline, cinnoline, phthalazine, naphthyridine and pteridine groups.

Examples of polycyclic aryl and heteroaryl groups containing an aromatic ring and a non-aromatic ring include tetrahydronaphthalene, tetrahydroisoquinoline, tetrahydroquinoline, dihydrobenzthiene, dihydrobenzfuran, 2,3-dihydro-benzo[1,4]dioxine, benzo[1,3]dioxole, 4,5,6,7-tetrahydrobenzofuran, indoline and indane groups.

Examples of carbocyclic aryl groups include phenyl, naphthyl, indenyl, and tetrahydronaphthyl groups.

Examples of non-aromatic heterocyclic groups include unsubstituted or substituted (by one or more groups R¹⁰) heterocyclic groups having from 3 to 12 ring members, typically 4 to 12 ring members, and more usually from 5 to 10 ring members. Such groups can be monocyclic or bicyclic, for example, and typically have from 1 to 5 heteroatom ring members (more usually 1,2, 3 or 4 heteroatom ring members) typically selected from nitrogen, oxygen and sulphur.

When sulphur is present, it may, where the nature of the adjacent atoms and groups permits, exist as —S—, —S(O)— or —S(O)₂—.

The heterocylic groups can contain, for example, cyclic ether moieties (e.g. as in tetrahydrofuran and dioxane), cyclic thioether moieties (e.g. as in tetrahydrothiophene and dithiane), cyclic amine moieties (e.g. as in pyrrolidine), cyclic amide moieties (e.g. as in pyrrolidone), cyclic urea moieties (e.g. as in imidazolidin-2-one), cyclic thiourea moieties, cyclic thioamides, cyclic thioesters, cyclic ester moieties (e.g. as in butyrolactone), cyclic sulphones (e.g. as in sulpholane and sulpholene), cyclic sulphoxides, cyclic sulphonamides and combinations thereof (e.g. morpholine and thiomorpholine and its S-oxide and S,S-dioxide).

Examples of monocyclic non-aromatic heterocyclic groups include 5-, 6- and 7-membered monocyclic heterocyclic groups. Particular examples include morpholine, thiomorpholine and its S-oxide and S,S-dioxide (particularly thiomorpholine), piperidine (e.g. 1-piperidinyl, 2-piperidinyl 3-piperidinyl and 4-piperidinyl), N-alkyl piperidines such as N-methyl piperidine, piperidone, pyrrolidine (e.g. 1-pyrrolidinyl, 2-pyrrolidinyl and 3-pyrrolidinyl), pyrrolidone, azetidine, pyran (2H-pyran or 4H-pyran), dihydrothiophene, dihydropyran, dihydrofuran, dihydrothiazole, tetrahydrofuran, tetrahydrothiophene, dioxane, tetrahydropyran (e.g. 4-tetrahydro pyranyl), imidazoline, imidazolidinone, oxazoline, thiazoline, 2-pyrazoline, pyrazolidine, piperazone, piperazine, and N-alkyl piperazines such as N-methyl piperazine, N-ethyl piperazine and N-isopropylpiperazine. In general, preferred non-aromatic heterocyclic groups include piperidine, pyrrolidine, azetidine, morpholine, piperazine and N-alkyl piperazines.

Examples of non-aromatic carbocyclic groups include cycloalkane groups such as cyclohexyl and cyclopentyl, cycloalkenyl groups such as cyclopentenyl, cyclohexenyl, cycloheptenyl and cyclooctenyl, as well as cyclohexadienyl, cyclooctatetraene, tetrahydronaphthenyl and decalinyl.

Preferred non-aromatic carbocyclic groups are monocyclic rings and most preferably saturated monocyclic rings.

Typical examples are three, four, five and six membered saturated carbocyclic rings, e.g. optionally substituted cyclopentyl and cyclohexyl rings.

One sub-set of non-aromatic carbocyclic groups includes unsubstituted or substituted (by one or more groups R¹⁰) monocyclic groups and particularly saturated monocyclic groups, e.g. cycloalkyl groups. Examples of such cycloalkyl groups include cyclopropyl, cyclobutyl, cyclopentyl, cyclohexyl and cycloheptyl; more typically cyclopropyl, cyclobutyl, cyclopentyl and cyclohexyl, particularly cyclohexyl.

Further examples of non-aromatic cyclic groups include bridged ring systems such as bicycloalkanes and azabicycloalkanes although such bridged ring systems are generally less preferred. By “bridged ring systems” is meant ring systems in which two rings share more than two atoms, see for example Advanced Organic Chemistry, by Jerry March, 4^{th} Edition, Wiley Interscience, pages 131-133, 1992. Examples of bridged ring systems include bicyclo[2.2.1]heptane, aza-bicyclo[2.2.1]heptane, bicyclo[2.2.2]octane, aza-bicyclo[2.2.2]octane, bicyclo[3.2.1]octane and aza-bicyclo[3.2.1]octane.

Where reference is made herein to carbocyclic and heterocyclic groups, the carbocyclic or heterocyclic ring can, unless the context indicates otherwise, be unsubstituted or substituted by one or more substituent groups R¹⁰ selected from halogen, hydroxy, trifluoromethyl, cyano, nitro, carboxy, amino, mono- or di-C₁₋₄ hydrocarbylamino, carbocyclic and heterocyclic groups having from 3 to 12 ring members; a group R^{a}-R^{b} wherein R^{a} is a bond, O, CO, X¹C(X²), C(X²)X¹, X¹C(X²)X¹, S, SO, SO₂, NR^{c}, SO₂NR^{c} or NR^{c}SO₂; and R^{b} is selected from hydrogen, carbocyclic and heterocyclic groups having from 3 to 12 ring members, and a C₁₋₈ hydrocarbyl group optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹;

R^{c} is selected from hydrogen and C₁₋₄ hydrocarbyl; and

X¹ is O, S or NR^{c} and X² is ═O, ═S or ═NR^{c}.

Where the substituent group R¹⁰ comprises or includes a carbocyclic or heterocyclic group, the said carbocyclic or heterocyclic group may be unsubstituted or may itself be substituted with one or more further substituent groups R¹⁰. In one sub-group of compounds of the formula (I) as herein defined, such further substituent groups R¹⁰ may include carbocyclic or heterocyclic groups, which are typically not themselves further substituted. In another sub-group of compounds of the formula (I) as herein defined, the said further substituents do not include carbocyclic or heterocyclic groups but are otherwise selected from the groups listed above in the definition of R¹⁰.

The substituents R¹⁰ may be selected such that they contain no more than 20 non-hydrogen atoms, for example, no more than 15 non-hydrogen atoms, e.g. no more than 12, or 10, or 9, or 8, or 7, or 6, or 5 non-hydrogen atoms.

One sub-group of substituents R¹⁰ is represented by R^{10a} which consists of substituents selected from halogen, hydroxy, trifluoromethyl, cyano, nitro, carboxy, amino, mono- or di-C₁₋₄ hydrocarbylamino, carbocyclic and heterocyclic groups having from 3 to 7 ring members; a group R^{a}-R^{b} wherein R^{a} is a bond, O, CO, OC(O), NR^{c}C(O), OC(NR^{c}), C(O)O, C(O)NR^{c}, OC(O)O, NR^{c}C(O)O, OC(O)NR^{c}, NR^{c}C(O)NR^{c}, S. SO, SO₂, NR^{c}, SO₂NR^{c} or NR^{c}SO₂; and R^{b} is selected from hydrogen, carbocyclic and heterocyclic groups having from 3 to 7 ring members, and a C₁₋₈ hydrocarbyl group optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 7 ring members and wherein one or more carbon atoms of the C₁₋₈ hydrocarbyl group may optionally be replaced by O, S, SO, SO₂, NR^{c}, OC(O), NR^{c}C(O), OC(NR^{c}), C(O)O, C(O)NR^{c}, OC(O)O, NR^{c}C(O)O, OC(O)NR^{c} or NR^{c}C(O)NR^{c};

R^{c} is selected from hydrogen and C₁₋₄ hydrocarbyl.

Another sub-group of substituents R¹⁰ is represented by R^{10b} which consists of substituents selected from halogen, hydroxy, trifluoromethyl, cyano, amino, mono- or di-C₁₋₄ alkylamino, cyclopropylamino, carbocyclic and heterocyclic groups having from 3 to 7 ring members; a group R^{a}-R^{b} wherein R^{a} is a bond, O, CO, OC(O), NR^{c}C(O), OC(NR^{c}), C(O)O, C(O)NR^{c}, S. SO, SO₂, NR^{c}, SO₂NR^{c} or NR^{c}SO₂; and R^{b} is selected from hydrogen, carbocyclic and heterocyclic groups having from 3 to 7 ring members, and a C₁₋₈ hydrocarbyl group optionally substituted by one or more substituents selected from hydroxy, oxo, halogen, cyano, amino, mono- or di-C₁₋₄ alkylamino, carbocyclic and heterocyclic groups having from 3 to 7 ring members and wherein one or more carbon atoms of the C₁₋₈ hydrocarbyl group may optionally be replaced by O, S, SO, SO₂ or NR^{c}; provided that R^{a} is not a bond when R^{b} is hydrogen; and

R^{c} is selected from hydrogen and C₁₋₄ alkyl.
A further sub-group of substituents R¹⁰ is represented by R^{10c} which consists of substituents selected from:
halogen,
hydroxy,
trifluoromethyl,
cyano,
amino, mono- or di-C₁₋₄ alkylamino,
cyclopropylamino,
monocyclic carbocyclic and heterocyclic groups having from 3 to 7 ring members of which 0, 1 or 2 are selected from O, N and S and the remainder are carbon atoms, wherein the monocyclic carbocyclic and heterocyclic groups are optionally substituted by one or more substituents selected from halogen, hydroxy, trifluoromethyl, cyano and methoxy; a group R^{a}-R^{b};
R^{a} is a bond, O, CO, OC(O), NR^{c}C(O), OC(NR^{c}), C(O)O, C(O)NR^{c}, S, SO, SO₂, NR^{c}, SO₂NR^{c} or NR^{c}SO₂;
R^{b} is selected from hydrogen, monocyclic carbocyclic and heterocyclic groups having from 3 to 7 ring members of which 0, 1 or 2 are selected from O, N and S and the remainder are carbon atoms, wherein the monocyclic carbocyclic and heterocyclic groups are optionally substituted by one or more substituents selected from halogen, hydroxy, trifluoromethyl, cyano and methoxy;
and R is further selected from a C₁₋₈ hydrocarbyl group optionally substituted by one or more substituents selected from hydroxy, oxo, halogen, cyano, amino, mono- or di-C₁₋₄ alkylamino, monocyclic carbocyclic and heterocyclic groups having from 3 to 7 ring members of which 0, 1 or 2 are selected from O, N and S and the remainder are carbon atoms, wherein the monocyclic carbocyclic and heterocyclic groups are optionally substituted by one or more substituents selected from halogen, hydroxy, trifluoromethyl, cyano and methoxy, and wherein one or two carbon atoms of the C₁₋₈ hydrocarbyl group may optionally be replaced by O, S or NR^{c}; provided that R^{a} is not a bond when R^{b} is hydrogen; and
R^{c} is selected from hydrogen and C₁₋₄ alkyl.
Where the carbocyclic and heterocyclic groups have a pair of substituents on adjacent ring atoms, the two substituents may be linked so as to form a cyclic group. For example, an adjacent pair of substituents on adjacent carbon atoms of a ring may be linked via one or more heteroatoms and optionally substituted alkylene groups to form a fused oxa-, dioxa-, aza-, diaza- or oxa-aza-cycloalkyl group. Examples of such linked substituent groups include:

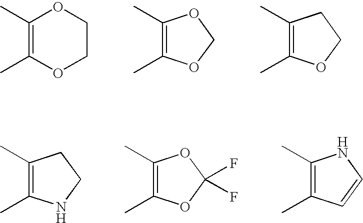

Examples of halogen substituents include fluorine, chlorine, bromine and iodine. Fluorine and chlorine are particularly preferred.

In the definition of the compounds of the formula (I) above and as used hereinafter, the term “hydrocarbyl” is a generic term encompassing aliphatic, alicyclic and aromatic groups having an all-carbon backbone and consisting of carbon and hydrogen atoms, except where otherwise stated.

In certain cases, as defined herein, one or more of the carbon atoms making up the carbon backbone may be replaced by a specified atom or group of atoms. Examples of hydrocarbyl groups include alkyl, cycloalkyl, cycloalkenyl, carbocyclic aryl, alkenyl, alkynyl, cycloalkylalkyl, cycloalkenylalkyl, and carbocyclic aralkyl, aralkenyl and aralkynyl groups. Such groups can be unsubstituted or, where stated, can be substituted by one or more substituents as defined herein. The examples and preferences expressed below apply to each of the hydrocarbyl substituent groups or hydrocarbyl-containing substituent groups referred to in the various definitions of substituents for compounds of the formula (I) and sub-groups thereof as defined herein unless the context indicates otherwise.

Generally by way of example, the hydrocarbyl groups can have up to eight carbon atoms, unless the context requires otherwise. Within the sub-set of hydrocarbyl groups having 1 to 8 carbon atoms, particular examples are C₁₋₆ hydrocarbyl groups, such as C₁₋₄ hydrocarbyl groups (e.g. C₁₋₃ hydrocarbyl groups or C₁₋₂ hydrocarbyl groups), specific examples being any individual value or combination of values selected from C₁, C₂, C₃, C₄, C₅, C₆, C₇ and C₈ hydrocarbyl groups.

The term “saturated hydrocarbyl”, whether used alone or together with a suffix such as “oxy” (e.g. as in “hydrocarbyloxy”), refers to a non-aromatic hydrocarbon group containing no multiple bonds such as C═C and C≡C.

Particular hydrocarbyl groups are saturated hydrocarbyl groups such as alkyl and cycloalkyl groups as defined herein.

The term “alkyl” covers both straight chain and branched chain alkyl groups. Examples of alkyl groups include methyl, ethyl, propyl, isopropyl, n-butyl, isobutyl, tert-butyl, n-pentyl, 2-pentyl, 3-pentyl, 2-methyl butyl, 3-methyl butyl, and n-hexyl and its isomers. Within the sub-set of alkyl groups having 1 to 8 carbon atoms, particular examples are C₁₋₆ alkyl groups, such as C₁₋₄ alkyl groups (e.g. C₁₋₃ alkyl groups or C₁₋₂ alkyl groups).

Examples of cycloalkyl groups are those derived from cyclopropane, cyclobutane, cyclopentane, cyclohexane and cycloheptane. Within the sub-set of cycloalkyl groups the cycloalkyl group will have from 3 to 8 carbon atoms, particular examples being C₃₋₆ cycloalkyl groups.

Examples of alkenyl groups include, but are not limited to, ethenyl (vinyl), 1-propenyl, 2-propenyl (allyl), isopropenyl, butenyl, buta-1,4-dienyl, pentenyl, and hexenyl. Within the sub-set of alkenyl groups the alkenyl group will have 2 to 8 carbon atoms, particular examples being C₂₋₆ alkenyl groups, such as C₂₋₄ alkenyl groups.

Examples of cycloalkenyl groups include, but are not limited to, cyclopropenyl, cyclobutenyl, cyclopentenyl, cyclopentadienyl and cyclohexenyl. Within the sub-set of cycloalkenyl groups the cycloalkenyl groups have from 3 to 8 carbon atoms, and particular examples are C₃₋₆ cycloalkenyl groups.

Examples of alkynyl groups include, but are not limited to, ethynyl and 2-propynyl (propargyl) groups. Within the sub-set of alkynyl groups having 2 to 8 carbon atoms, particular examples are C₂₋₆ alkynyl groups, such as C₂₋₄ alkynyl groups.

Examples of carbocyclic aryl groups include substituted and unsubstituted phenyl, naphthyl, indane and indene groups.

Examples of cycloalkylalkyl, cycloalkenylalkyl, carbocyclic aralkyl, aralkenyl and aralkynyl groups include phenethyl, benzyl, styryl, phenylethynyl, cyclohexylmethyl, cyclopentylmethyl, cyclobutylmethyl, cyclopropylmethyl and cyclopentenylmethyl groups. When present, and where stated, a hydrocarbyl group can be optionally substituted by one or more substituents selected from hydroxy, oxo, alkoxy, carboxy, halogen, cyano, nitro, amino, mono- or di-C₁₋₄ hydrocarbylamino, and monocyclic or bicyclic carbocyclic and heterocyclic groups having from 3 to 12 (typically 3 to 10 and more usually 5 to 10) ring members. Preferred substituents include halogen such as fluorine. Thus, for example, the substituted hydrocarbyl group can be a partially fluorinated or perfluorinated group such as difluoromethyl or trifluoromethyl. In one embodiment preferred substituents include monocyclic carbocyclic and heterocyclic groups having 3-7 ring members.

Where stated, one or more carbon atoms of a hydrocarbyl group may optionally be replaced by O, S, SO, SO₂, NR^{c}, X¹C(X²), C(X²)X¹ or X¹C(X²)X¹ (or a sub-group thereof) wherein X¹ and X² are as hereinbefore defined, provided that at least one carbon atom of the hydrocarbyl group remains. For example, 1, 2, 3 or 4 carbon atoms of the hydrocarbyl group may be replaced by one of the atoms or groups listed, and the replacing atoms or groups may be the same or different. In general, the number of linear or backbone carbon atoms replaced will correspond to the number of linear or backbone atoms in the group replacing them. Examples of groups in which one or more carbon atom of the hydrocarbyl group have been replaced by a replacement atom or group as defined above include ethers and thioethers (C replaced by O or S), amides, esters, thioamides and thioesters (C—C replaced by X¹C(X²) or C(X²)X¹), sulphones and sulphoxides (C replaced by SO or SO₂), amines (C replaced by NR^{c}). Further examples include ureas, carbonates and carbamates (C—C—C replaced by X¹C(X²)X¹).

Where an amino group has two hydrocarbyl substituents, they may, together with the nitrogen atom to which they are attached, and optionally with another heteroatom such as nitrogen, sulphur, or oxygen, link to form a ring structure of 4 to 7 ring members.

The term “aza-cycloalkyl” as used herein refers to a cycloalkyl group in which one of the carbon ring members has been replaced by a nitrogen atom. Thus examples of aza-cycloalkyl groups include piperidine and pyrrolidine. The term “oxa-cycloalkyl” as used herein refers to a cycloalkyl group in which one of the carbon ring members has been replaced by an oxygen atom. Thus examples of oxa-cycloalkyl groups include tetrahydrofuran and tetrahydropyran. In an analogous manner, the terms “diaza-cycloalkyl”, “dioxa-cycloalkyl” and “aza-oxa-cycloalkyl” refer respectively to cycloalkyl groups in which two carbon ring members have been replaced by two nitrogen atoms, or by two oxygen atoms, or by one nitrogen atom and one oxygen atom.

The definition “R^{a}-R^{b}” as used herein, either with regard to substituents present on a carbocyclic or heterocyclic moiety, or with regard to other substituents present at other locations on the compounds of the formula (I) as defined herein, includes inter alia compounds wherein R^{a} is selected from a bond, O, CO, OC(O), SC(O), NR^{c}C(O), OC(S), SC(S), NR^{c}C(S), OC(NR^{c}), SC(NR^{c}), NR^{c}C(NR^{c}), C(O)O, C(O)S, C(O)NR^{c}, C(S)O, C(S)S, C(S)NR^{c}, C(NR^{c})O, C(NR^{c})S, C(NR^{c})NR^{c}, OC(O)O, SC(O)O, NR^{c}C(O)O, OC(S)O, SC(S)O, NR^{c}C(S)O, OC(NR^{c})O, SC(NR^{c})O, NR^{c}C(NR^{c})O, OC(O)S, SC(O)S, NR^{c}C(O)S, OC(S)S, SC(S)S, NR^{c}C(S)S, OC(NR^{c})S, SC(NR^{c})S, NR^{c}C(NR^{c})S, OC(O)NR^{c}, SC(O)NR^{c}, NR^{c}C(O)NR^{c}, OC(S)NR^{c}, SC(S)NR^{c}, NR^{c}C(S)NR^{c}, OC(NR^{c})NR^{c}, SC(NR^{c})NR^{c}, NR^{c}C(NR^{c}NR^{c}, S. SO, SO₂, NR^{c}, SO₂NR^{c} and NR^{c}SO₂ wherein R^{c} is as hereinbefore defined.

The moiety R^{b} can be hydrogen or it can be a group selected from carbocyclic and heterocyclic groups having from 3 to 12 ring members (typically 3 to 10 and more usually from 5 to 10), and a C₁₋₈ hydrocarbyl group optionally substituted as hereinbefore defined. Examples of hydrocarbyl, carbocyclic and heterocyclic groups are as set out above.

When R^{a} is O and R^{b} is a C₁₋₈ hydrocarbyl group, R^{a} and R^{b} together form a hydrocarbyloxy group. Preferred hydrocarbyloxy groups include saturated hydrocarbyloxy such as alkoxy (e.g. C₁₋₆ alkoxy, more usually C₁₋₄ alkoxy such as ethoxy and methoxy, particularly methoxy), cycloalkoxy (e.g. C₃₋₆ cycloalkoxy such as cyclopropyloxy, cyclobutyloxy, cyclopentyloxy and cyclohexyloxy) and cycloalkyalkoxy (e.g. C₃₋₆ cycloalkyl-C₁₋₂ alkoxy such as cyclopropylmethoxy).

The hydrocarbyloxy groups can be substituted by various substituents as defined herein. For example, the alkoxy groups can be substituted by halogen (e.g. as in difluoromethoxy and trifluoromethoxy), hydroxy (e.g. as in hydroxyethoxy), C₁₋₂ alkoxy (e.g. as in methoxyethoxy), hydroxy-C₁₋₂ alkyl (as in hydroxyethoxyethoxy) or a cyclic group (e.g. a cycloalkyl group or non-aromatic heterocyclic group as hereinbefore defined). Examples of alkoxy groups bearing a non-aromatic heterocyclic group as a substituent are those in which the heterocyclic group is a saturated cyclic amine such as morpholine, piperidine, pyrrolidine, piperazine, C₁₋₄-alkyl-piperazines, C₃₋₇-cycloalkyl-piperazines, tetrahydropyran or tetrahydrofuran and the alkoxy group is a C₁₋₄ alkoxy group, more typically a C₁₋₃ alkoxy group such as methoxy, ethoxy or n-propoxy.

Alkoxy groups may be substituted by, for example, a monocyclic group such as pyrrolidine, piperidine, morpholine and piperazine and N-substituted derivatives thereof such as N-benzyl, N—C₁₋₄ acyl and N—C₁₋₄ alkoxycarbonyl. Particular examples include pyrrolidinoethoxy, piperidinoethoxy and piperazinoethoxy.

When R^{a} is a bond and R^{b} is a C₁₋₈ hydrocarbyl group, examples of hydrocarbyl groups R^{a}-R^{b} are as hereinbefore defined. The hydrocarbyl groups may be saturated groups such as cycloalkyl and alkyl and particular examples of such groups include methyl, ethyl and cyclopropyl. The hydrocarbyl (e.g. alkyl) groups can be substituted by various groups and atoms as defined herein. Examples of substituted alkyl groups include alkyl groups substituted by one or more halogen atoms such as fluorine and chlorine (particular examples including bromoethyl, chloroethyl, difluoromethyl, 2,2,2-trifluoroethyl and perfluoroalkyl groups such as trifluoromethyl), or hydroxy (e.g. hydroxymethyl and hydroxyethyl), C₁₋₈ acyloxy (e.g. acetoxymethyl and benzyloxymethyl), amino and mono- and dialkylamino (e.g. aminoethyl, methylaminoethyl, dimethylaminomethyl, dimethylaminoethyl and tert-butylaminomethyl), alkoxy (e.g. C₁₋₂ alkoxy such as methoxy-as in methoxyethyl), and cyclic groups such as cycloalkyl groups, aryl groups, heteroaryl groups and non-aromatic heterocyclic groups as hereinbefore defined).

Particular examples of alkyl groups substituted by a cyclic group are those wherein the cyclic group is a saturated cyclic amine such as morpholine, piperidine, pyrrolidine, piperazine, C₁₋₄-alkyl-piperazines, C₃₋₇-cycloalkyl-piperazines, tetrahydropyran or tetrahydrofuran and the alkyl group is a C₁₋₄ alkyl group, more typically a C₁₋₃ alkyl group such as methyl, ethyl or n-propyl. Specific examples of alkyl groups substituted by a cyclic group include pyrrolidinomethyl, pyrrolidinopropyl, morpholinomethyl, morpholinoethyl, morpholinopropyl, piperidinylmethyl, piperazinomethyl and N-substituted forms thereof as defined herein.

Particular examples of alkyl groups substituted by aryl groups and heteroaryl groups include benzyl, phenethyl and pyridylmethyl groups.

When R^{a} is SO₂NR^{c}, R^{b} can be, for example, hydrogen or an optionally substituted C₁₋₈ hydrocarbyl group, or a carbocyclic or heterocyclic group. Examples of R^{a}-R^{b} where R^{a} is SO₂NR^{c} include aminosulphonyl, C₁₋₄ alkylaminosulphonyl and di-C₁₋₄ alkylaminosulphonyl groups, and sulphonamides formed from a cyclic amino group such as piperidine, morpholine, pyrrolidine, or an optionally N-substituted piperazine such as N-methyl piperazine.

Examples of groups R^{a}-R^{b} where R^{a} is SO₂ include alkylsulphonyl, heteroarylsulphonyl and arylsulphonyl groups, particularly monocyclic aryl and heteroaryl sulphonyl groups. Particular examples include methylsulphonyl, phenylsulphonyl and toluenesulphonyl.

When R^{a} is NR^{c}, R^{b} can be, for example, hydrogen or an optionally substituted C₁₋₈ hydrocarbyl group, or a carbocyclic or heterocyclic group. Examples of R^{a}-R^{b} where R^{a} is NR^{c} include amino, C₁₋₄ alkylamino (e.g. methylamino, ethylamino, propylamino, isopropylamino, tert-butylamino), di-C₁₋₄ alkylamino (e.g. dimethylamino and diethylamino) and cycloalkylamino (e.g. cyclopropylamino, cyclopentylamino and cyclohexylamino).

### Specific Embodiments of and Preferences for A, E, T, J¹, J² and R¹ to R¹⁰

In formula (I) as defined herein, T can be nitrogen or a group CR⁵ and J¹-J² can represent a group selected from N═C(R⁶), (R⁷)C═N, (R⁸)N—C(O), (R⁸)₂C—C(O) and (R⁷)C═C(R⁶). Thus the bicyclic group can take the form of, for example:
a purine (T is N. J¹-J² is N═C(R⁶));
a 3H-imidazo[4,5-b]pyridine (T is CR⁵, J¹-J² is N═C(R⁶));
a 7H-pyrrolo[2,3-d]pyrimidine (T is N. J¹-J² is (R⁷)C═C(R⁶));
a 1H-pyrrolo[2,3-b]pyridine (T is CR⁵, J¹-J² is (R⁷)C═C(R⁶));
a 5,7-dihydro-pyrrolo[2,3-d]pyrimidin-6-one (T is N. J¹-J² is (R⁸)₂C—C(O));
a 3H-[1,2,3]triazolo[4,5-d]pyrimidine (T is N. J¹-J² is N═N);
a 3H-[1,2,3]triazolo[4,5-b]pyridine (T is CR⁵, J¹-J² is N═N);
a 7,9-dihydro-purin-8-one (T is N. J¹-J² is (R⁸)N—C(O));
a 1H-pyrazolo[3,4-d]pyrimidine (T is N. J¹-J² is (R⁷)C═N); or
a pyrazolo[3,4-b]pyridine (T is CR⁵, J¹-J² is (R⁷)C═N).

R⁴ is selected from hydrogen; halogen; C₁₋₆ hydrocarbyl optionally substituted by halogen, hydroxy or C₁₋₂ alkoxy; cyano; CONH₂; CONHR⁹; CF₃; NH₂; NHCOR⁹ and NHCONHR⁹. Typically, R⁴ is selected from hydrogen, halogen, C₁₋₅ saturated hydrocarbyl, cyano and CF₃. More typically, R⁴ is selected from hydrogen, chlorine, fluorine and methyl, and preferably R⁴ is hydrogen.

R⁵ is selected from hydrogen; halogen; C₁₋₆ hydrocarbyl optionally substituted by halogen, hydroxy or C₁₋₂ alkoxy; cyano; CONH₂; CONHR⁹; CF₃; NH₂; NHCOR⁹ and NHCONHR⁹. Typically, R⁵ is selected from hydrogen, halogen, C₁₋₅ saturated hydrocarbyl, cyano and CF₃. Preferably, R⁵ is selected from hydrogen, chlorine, fluorine and methyl, and more preferably R⁵ is hydrogen.

R⁶ is selected from hydrogen; halogen; C₁₋₆ hydrocarbyl optionally substituted by halogen, hydroxy or C₁₋₂ alkoxy; cyano; CONH₂; CONHR⁹; CF₃; NH₂; NHCOR⁹ and NHCONHR⁹. Typically, R⁵ is selected from hydrogen, halogen, C₁₋₅ saturated hydrocarbyl, cyano and CF₃. More typically R⁶ is selected from hydrogen, chlorine, fluorine and methyl, and preferably R⁵ is hydrogen.

R⁷ is selected from hydrogen; halogen; C₁₋₆ hydrocarbyl optionally substituted by halogen, hydroxy or C₁₋₂ alkoxy; cyano; CONH₂; CONHR⁹; CF₃; NH₂; NHCOR⁹ and NHCONHR⁹. More typically R⁷ is selected from hydrogen, halogen, C₁₋₅ saturated hydrocarbyl, cyano and CF₃. Preferably, R⁷ is selected from hydrogen, chlorine, fluorine and methyl, and more preferably R⁷ is hydrogen.

R⁸ is selected from hydrogen, halogen, C₁₋₅ saturated hydrocarbyl, cyano, CONH₂, CONHR⁹, CF₃, NH₂, NHCOR⁹ and NHCONHR⁹. Typically, R⁶ is selected from hydrogen, halogen, C₁₋₅ saturated hydrocarbyl, cyano and CF₃. More typically, R⁸ is selected from hydrogen, chlorine, fluorine and methyl, and preferably R⁸ is hydrogen.

R⁹ is phenyl or benzyl each optionally substituted as defined herein. Particular groups R⁹ are phenyl and benzyl groups that are unsubstituted or are substituted with a solubilising group such as an alkyl or alkoxy group bearing an amino, substituted amino, carboxylic acid or sulphonic acid group. Particular examples of solubilising groups include amino-C₁₋₄-alkyl, mono-C₁₋₂-alkylamino-C₁₋₄-alkyl, di-C₁₋₂-alkylamino-C₁₋₄-alkyl, amino-C₁₋₄-alkoxy, mono-C₁₋₂-alkylamino-C₁₋₄-alkoxy, di-C₁₋₂-alkylamino-C₁₋₄-alkoxy, piperidinyl-C₁₋₄-alkyl, piperazinyl-C₁₋₄-alkyl, morpholinyl-C₁₋₄-alkyl, piperidinyl-C₁₋₄-alkoxy, piperazinyl-C₁₋₄-alkoxy and morpholinyl-C₁₋₄-alkoxy.

A is a saturated hydrocarbon linker group containing from 1 to 7 carbon atoms, the linker group having a maximum chain length of 5 atoms extending between R¹ and NR²R³ and a maximum chain length of 4 atoms extending between E and NR²R³. Within these constraints, the moieties E and R¹ can each be attached at any location on the group A.

The term “maximum chain length” as used herein refers to the number of atoms lying directly between the two moieties in question, and does not take into account any branching in the chain or any hydrogen atoms that may be present. For example, in the structure A shown below:

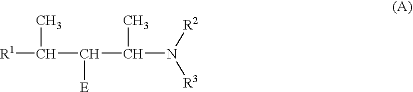

the chain length between R¹ and NR²R³ is 3 atoms whereas the chain length between E and NR²R³ is 2 atoms.

In general it is presently preferred that the linker group has a maximum chain length of 3 atoms (more preferably 1 or 2 atoms, and most preferably 2 atoms) extending between R¹ and NR²R³.

It is preferred that the linker group has a maximum chain length of 4 atoms, more typically 3 atoms, extending between E and NR²R³.

In one particularly preferred group of compounds, the linker group has a chain length of 1, 2 or 3 atoms extending between R¹ and NR²R³ and a chain length of 1, 2 or 3 atoms extending between E and NR²R³.

One of the carbon atoms in the linker group may optionally be replaced by an oxygen or nitrogen atom. When present, the oxygen or nitrogen atom preferably is linked directly to the group E.

When a nitrogen atom or oxygen atom are present, it is preferred that the nitrogen or oxygen atom and the NR²R³ group are spaced apart by at least two intervening carbon atoms.

In one particular group of compounds within formula (I) as defined herein, the linker atom linked directly to the group E is a carbon atom and the linker group A has an all-carbon skeleton.

The carbon atoms of the linker group A may optionally bear one or more substituents selected from oxo, fluorine and hydroxy, provided that the hydroxy group is not located at a carbon atom α with respect to the NR²R³ group, and provided also that the oxo group is located at a carbon atom α with respect to the NR²R³ group. Typically, the hydroxy group, if present, is located at a position β with respect to the NR²R³ group. In general, no more than one hydroxy group will be present. Where fluorine atoms are present, they may be present in a difluoromethylene or trifluoromethyl group, for example.

It will be appreciated that that when an oxo group is present at the carbon atom adjacent the NR²R³ group, the compound of the formula (I) will be an amide.

In one embodiment of the invention, no fluorine atoms are present in the linker group A.

In another embodiment of the invention, no hydroxy groups are present in the linker group A.

In a further embodiment, no oxo group is present in the linker group A.

In one group of compounds of the formula (I) neither hydroxy groups nor fluorine atoms are present in the linker group A, e.g. the linker group A is unsubstituted.

Preferably, when a carbon atom in the linker group A is replaced by a nitrogen atom, the group A bears no more than one hydroxy substituent and more preferably bears no hydroxy substituents.

In another group of compounds for use according to the invention, the linker group A can have a branched configuration at the carbon atom attached to the NR²R³ group. For example, the carbon atom attached to the NR²R³ group can be attached to a pair of gem-dimethyl groups.

In one particular group of compounds of the formula (I) as defined herein, the portion R¹-A-NR²R³ of the compound is represented by the formula R¹-(G)ₖ-(CH₂)ₘ—X—(CH₂)ₙ—(CR⁶R⁷)ₚ—NR²R³ wherein G is NH, NMe or O; X is attached to the group E and is selected from (CH₂)ⱼ—CH, (CH₂)ⱼ—N, O—CH and (NH)ⱼ—CH;, j is 0 or 1, k is 0 or 1, m is 0 or 1, n is 0, 1, 2, or 3 and p is 0 or 1, and the sum of j, k, m, n and p does not exceed 4; and R⁶ and R⁷ are the same or different and are selected from methyl and ethyl, or CR⁶R⁷ forms a cyclopropyl group.

One particular group CR⁶R⁷ is C(CH₃)₂.

Preferably X is (CH₂)ⱼ—CH.

Particular configurations are those wherein:
k is 0, m is 0 or 1, n is 0, 1, 2 or 3 and p is 0;
k is O, m is 0 or 1, n is 0, 1 or 2 and p is 1;
X is (CH₂)ⱼ—CH, k is l, m is 0, n is 0, 1, 2 or 3 and p is 0; and
X is (CH₂)ⱼ—CH, k is l, m is 0, n is 0, 1 or 2 and p is 1.

In another embodiment, the portion R¹-A-NR²R³ of the compound is represented by the formula R¹—(CH₂)ₓ—X′—(CH₂)_{y}—NR²R³ wherein x is 0, 1 or 2, y is 0, 1 or 2 provided that the sum of x and y does not exceed 4; X¹ is attached to the group E and is a group C(R^{x}) where (i) R^{x} is hydrogen or (ii) R^{x} together with R² constitutes an alkylene linking chain of up to 3 carbon atoms in length such that the moiety X′—(CH₂)_{y}—NR²R³ forms a 4 to 7 membered saturated heterocyclic ring.

In one group of compounds, R² and R³ are independently selected from hydrogen, C₁₋₄ hydrocarbyl and C₁₋₄ acyl wherein the hydrocarbyl and acyl groups are optionally substituted by one or more substituents selected from fluorine, hydroxy, amino, methylamino, dimethylamino, methoxy and a monocyclic or bicyclic aryl or heteroaryl group.

Within this group of compounds, R² and R³ may be independently selected from hydrogen, C₁₋₄ hydrocarbyl and C₁₋₄ acyl. Typically the hydrocarbyl group is an alkyl group, more usually a C₁, C₂ or C₃ alkyl group, for example a methyl group. In a particular sub-group of compounds, R² and R³ are independently selected from hydrogen and methyl and hence NR²R³ can be an amino, methylamino or dimethylamino group. In one embodiment, NR²R³ is an amino group. In another particular embodiment, NR²R³ is a methylamino group.

In another group of compounds, R² and R³ together with the nitrogen atom to which they are attached form a cyclic group selected from an imidazole group and a saturated monocyclic heterocyclic group having 4-7 ring members and optionally containing a second heteroatom ring member selected from O and N;

Within this group of compounds, is the sub-group wherein R² and R³ together with the nitrogen atom to which they are attached form a saturated monocyclic heterocyclic group having 4-7 ring members and optionally containing a second heteroatom ring member selected from O and N.

When NR²R³ forms a saturated monocyclic group, this may be substituted by one or more substituents independently selected from a group R¹⁰ as defined herein. More particularly the monocyclic heterocyclic group may be substituted by one or more C₁₋₄ alkyl groups. Alternatively, the monocyclic heterocyclic group may be unsubstituted.

The saturated monocyclic ring can be an azacycloalkyl group such as an azetidine, pyrrolidine, piperidine or azepane ring, and such rings are typically unsubstituted.

Alternatively, the saturated monocyclic ring can contain an additional heteroatom selected from O and N. and examples of such groups include morpholine and piperazine. Where an additional N atom is present in the ring, this can form part of an NH group or an N—C₁₋₄alkyl group such as an N-methyl, N-ethyl, N-propyl or N-isopropyl group.

In a further group of compounds, one of R² and R³ together with the nitrogen atom to which they are attached and one or more atoms from the linker group A form a saturated monocyclic heterocyclic group having 4-7 ring members and optionally containing a second heteroatom ring member selected from O and N.

Examples of such compounds include compounds wherein NR²R³ and A form a unit of the formula:

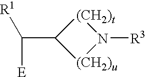

where t and u are each 0, 1, 2 or 3 provided that the sum of t and u falls within the range of 2 to 4.

Further examples of such compounds include compounds wherein NR²R³ and A form a group of the formula:

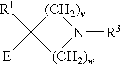

where v and w are each 0, 1, 2 or 3 provided that the sum of v and w falls within the range of 2 to 5. Particular examples of such compounds are those in which v and w are both 2.

Particular examples of the linker group A, together with their points of attachment to the groups R¹, E and NR²R³, are shown in Table 1 below.

TABLE 1

| | |
|---|---|
| 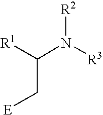 A1 | 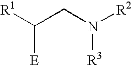 A2 |
| 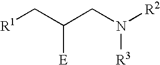 A3 | 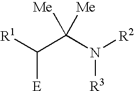 A4 |
| 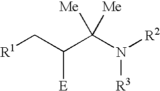 A5 | 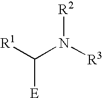 A6 |
| 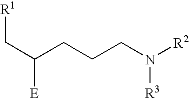 A7 | 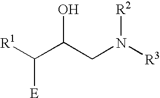 A8 |
| 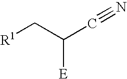 A9 | 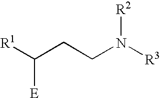 A10 |
| 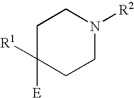 A11 | |

Currently preferred groups include A1, A2, A3, A10 and A11. Particularly preferred groups include A1 and A11.

In formula (I), E is a monocyclic or bicyclic carbocyclic or heterocyclic group or an acyclic group X-G wherein X is selected from CH₂, O S and NH and G is a C₁₋₄ alkylene chain wherein one of the carbon atoms is optionally replaced by O, S or NH.

When E is a monocyclic or bicyclic carbocyclic or heterocyclic group, it can be selected from the groups set out above in the section headed General Preferences and Definitions.

Particular cyclic groups E are monocyclic and bicyclic aryl and heteroaryl groups and, in particular, groups containing a six membered aromatic or heteroaromatic ring such as a phenyl, pyridine, pyrazine, pyridazine or pyrimidine ring, more particularly a phenyl, pyridine, pyrazine or pyrimidine ring, and more preferably a pyridine or phenyl ring.

Examples of bicyclic groups include benzo-fused and pyrido-fused groups wherein the group A and the pyrazole ring are both attached to the benzo- or pyrido-moiety.

In one embodiment, E is a monocyclic group.

Particular examples of monocyclic groups include monocyclic aryl and heteroaryl groups such as phenyl, thiophene, furan, pyrimidine, pyrazine and pyridine, phenyl being presently preferred.

Examples of non-aromatic monocyclic groups include cycloalkanes such as cylcohexane and cyclopentane, and nitrogen-containing rings such as piperidine, piperazine and piperazone.

One particular non-aromatic monocyclic group is a piperidine group and more particularly a piperidine group wherein the nitrogen atom of the piperidine ring is attached to the bicyclic group.

In one particular sub-group of compounds, E is selected from phenyl and piperidine groups.

It is preferred that the group A and the bicyclic group are attached to the group E in a meta or para relative orientation; i.e. A and the bicyclic group are not attached to adjacent ring members of the group E. Examples of groups such groups E include 1,4-phenylene, 1,3-phenylene, 2,5-pyridylene and 2,4-pyridylene, 1,4-piperidinyl, 1,4-piperindonyl, 1,4-piperazinyl, and 1,4-piperazonyl.

The groups E can be unsubstituted or can have up to 4 substituents R¹¹ which may be selected from the group R¹⁰ as hereinbefore defined. More typically however, the substituents R¹¹ are selected from hydroxy; CH₂CN, oxo (when E is non-aromatic); halogen (e.g. chlorine and bromine); trifluoromethyl; cyano; C₁₋₄ hydrocarbyloxy optionally substituted by C₁₋₂ alkoxy or hydroxy; and C₁₋₄ hydrocarbyl optionally substituted by C₁₋₂ alkoxy or hydroxy.

Typically, there are 0-3 substituents, more usually 0-2 substituents, for example 0 or 1 substituent. In one embodiment, the group E is unsubstituted.

The group E can be an aryl or heteroaryl group having five or six members and containing up to three heteroatoms selected from O, N and S, the group E being represented by the formula:

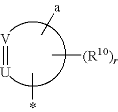

where * denotes the point of attachment to the bicyclic group, and “a” denotes the attachment of the group A;
r is 0, 1 or 2;
U is selected from N and CR^{12a}; and
V is selected from N and CR^{12b}; where R^{12a} and R^{12b} are the same or different and each is hydrogen or a substituent containing up to ten atoms selected from C, N, O, F, Cl and S provided that the total number of non-hydrogen atoms present in R^{12a} and R^{12b} together does not exceed ten;
or R^{12a} and R^{12b} together with the carbon atoms to which they are attached form an unsubstituted five or six membered saturated or unsaturated ring containing up to two heteroatoms selected from O and N; and
R¹⁰ is as hereinbefore defined.
In one particular group of compounds, E is a group:

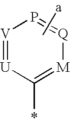

where * denotes the point of attachment to the pyrazole group, and “a” denotes the attachment of the group A;
P, Q and M are the same or different and are selected from N, CH and NCR¹⁰, provided that the group A is attached to a carbon atom; and U, V and R¹⁰ are as hereinbefore defined.

Examples of R^{12a} and R^{12b} include hydrogen and substituent groups R¹⁰ as hereinbefore defined having no more than ten non-hydrogen atoms. Particular examples of R^{12a} and R^{12b} include methyl, ethyl, propyl, isopropyl, cyclopropyl, cyclobutyl, cyclopentyl, fluorine, chlorine, methoxy, trifluoromethyl, hydroxymethyl, hydroxyethyl, methoxymethyl, difluoromethoxy, trifluoromethoxy, 2,2,2-trifluoroethyl, cyano, amino, methylamino, dimethylamino, CONH₂, CO₂Et, CO₂H, acetamido, azetidinyl, pyrrolidino, piperidine, piperazino, morpholino, methylsulphonyl, aminosulphonyl, mesylamino and trifluoroacetamido.

When U is CR^{12a} and/or V is CR^{12b} the atoms or groups in R^{12a} and R^{12b} that are directly attached to the carbon atom ring members C are preferably selected from H, O (e.g. as in methoxy), NH (e.g. as in amino and methylamino) and CH₂ (e.g. as in methyl and ethyl).

In another particular group of compounds for use according to the invention, E is a group:

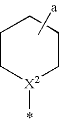

where X² is N or CH.

The group E can also be an acyclic group X-G wherein X is selected from CH₂, O S and NH and G is a C₁₋₄ alkylene chain wherein one of the carbon atoms is optionally replaced by O, S or NH.

Examples of acyclic groups X-G include NHCH₂CH₂, NHCH₂CH₂CH₂, NHCH₂CH₂CH₂CH₂, OCH₂CH₂, OCH₂CH₂CH₂, OCH₂CH₂CH₂ CH₂, SCH₂CH₂, SCH₂CH₂CH₂ and SCH₂CH₂CH₂ CH₂. Particular acyclic groups X-G are NHCH₂CH₂ and NHCH₂CH₂CH₂.

Particular examples of the linker group E, together with their points of attachment to the group A (^{a}) and the bicyclic group (*) are shown in Table 2 below.

TABLE 2

| | |
|---|---|
| 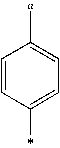 B1 | 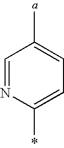 B2 |
| 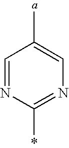 B3 | 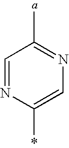 B4 |
| 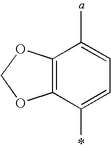 B5 | 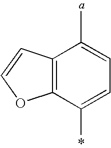 B6 |
| 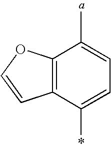 B7 | 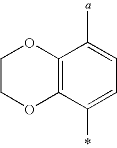 B8 |
| 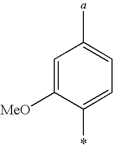 B9 | 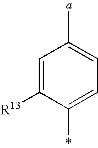 B10 |
| 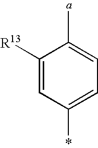 B11 | 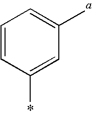 B12 |
| 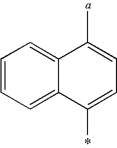 B13 | 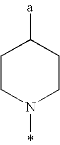 B14 |
| 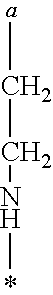 B15 | 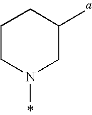 B16 |

In the table, the substituent group R¹³ is selected from methyl, chlorine, fluorine and trifluoromethyl.

The group R¹ is hydrogen or an aryl or heteroaryl group, wherein the aryl or heteroaryl group may be selected from the list of such groups set out in the section headed General Preferences and Definitions.

In one sub-group of compounds, R¹ is hydrogen.

In another sub-group of compounds, R¹ is an aryl or heteroaryl group.

When R¹ is aryl or heteroaryl, it can be monocyclic or bicyclic and, in one particular embodiment, is monocyclic. Particular examples of monocyclic aryl and heteroaryl groups are six membered aryl and heteroaryl groups containing up to 2 nitrogen ring members, and five membered heteroaryl groups containing up to 3 heteroatom ring members selected from O, S and N.

Examples of such groups include phenyl, naphthyl, thienyl, furan, pyrimidine and pyridine, with phenyl being presently preferred.

The aryl or heteroaryl group R¹ can be unsubstituted or substituted by up to 5 substituents, and examples of substituents are those listed in group R¹⁰ (or R^{10a}, R^{10b} or R^{10c}) above. Preferred substituents include hydroxy; C₁₋₄ acyloxy; fluorine; chlorine; bromine; trifluoromethyl; cyano; C₁₋₄ hydrocarbyloxy and C₁₋₄ hydrocarbyl each optionally substituted by C₁₋₂ alkoxy or hydroxy; C₁₋₄ acylamino; benzoylamino; pyrrolidinocarbonyl; piperidinocarbonyl; morpholinocarbonyl; piperazinocarbonyl; five and six membered heteroaryl groups containing one or two heteroatoms selected from N, O and S. the heteroaryl groups being optionally substituted by one or more C₁₋₄ alkyl substituents; phenyl; pyridyl; and phenoxy wherein the phenyl, pyridyl and phenoxy groups are each optionally substituted with 1, 2 or 3 substituents selected from C₁₋₂ acyloxy, fluorine, chlorine, bromine, trifluoromethyl, cyano, C₁₋₂ hydrocarbyloxy and C₁₋₂ hydrocarbyl each optionally substituted by methoxy or hydroxy.

Although up to 5 substituents may be present, more typically there are 0, 1, 2, 3 or 4 substituents, preferably 0, 1, 2 or 3, and more preferably 0, 1 or 2.

In one embodiment, the group R¹ is unsubstituted or substituted by up to 5 substituents selected from hydroxy; C₁₋₄ acyloxy; fluorine; chlorine; bromine; trifluoromethyl; cyano; C₁₋₄ hydrocarbyloxy and C₁₋₄ hydrocarbyl each optionally substituted by C₁₋₂ alkoxy or hydroxy.

In another embodiment, the group R¹ can have one or two substituents selected from fluorine, chlorine, trifluoromethyl, methyl and methoxy. When R¹ is a phenyl group, particular examples of substituent combinations include mono-chlorophenyl and dichlorophenyl.

When R¹ is a six membered aryl or heteroaryl group, a substituent may advantageously be present at the para position on the six-membered ring. Where a substituent is present at the para position, it is preferably larger in size than a fluorine atom.

In one embodiment, R¹ is selected from 4-fluorophenyl, 4-chlorophenyl and phenyl.

In formula (I), R⁴ is selected from hydrogen, halogen, C₁₋₅ saturated hydrocarbyl, cyano and CF₃. Preferred values for R⁴ include hydrogen and methyl.

In formula (I), R⁵ is selected from selected from hydrogen, halogen, C₁₋₅ saturated hydrocarbyl, cyano, CONH₂, CONHR⁹, CF₃, NH₂, NHCOR⁹ and NHCONHR⁹ where R⁹ is optionally substituted phenyl or benzyl.

More preferably, R⁵ is selected from selected from hydrogen, halogen, C₁₋₅ saturated hydrocarbyl, cyano, CF₃, NH₂, NHCOR⁹ and NHCONHR⁹ where R⁹ is optionally substituted phenyl or benzyl.

The group R⁹ is typically unsubstituted phenyl or benzyl, or phenyl or benzyl substituted by 1, 2 or 3 substituents selected from halogen; hydroxy; trifluoromethyl; cyano; carboxy; C₁₋₄ alkoxycarbonyl; C₁₋₄ acyloxy; amino; mono- or di-C₁₋₄ alkylamino; C₁₋₄ alkyl optionally substituted by halogen, hydroxy or C₁₋₂ alkoxy; C₁₋₄ alkoxy optionally substituted by halogen, hydroxy or C₁₋₂ alkoxy; phenyl, five and six membered heteroaryl groups containing up to 3 heteroatoms selected from O, N and S; and saturated carbocyclic and heterocyclic groups containing up to 2 heteroatoms selected from O, S and N.

Particular examples of the moiety R⁵ include hydrogen, fluorine, chlorine, bromine, methyl, ethyl, hydroxyethyl, methoxymethyl, cyano, CF₃, NH₂, NHCOR^{9a} and NHCONHR^{9a} where R^{9a} is phenyl or benzyl optionally substituted by hydroxy, C₁₋₄ acyloxy, fluorine, chlorine, bromine, trifluoromethyl, cyano, C₁₋₄ hydrocarbyloxy (e.g. alkoxy) and C₁₋₄ hydrocarbyl (e.g. alkyl) optionally substituted by C₁₋₂ alkoxy or hydroxy.

### Particular and Preferred Sub-Groups of the Formula (I)

In one embodiment of the formula (I) as defined herein, the compounds can be represented by the general formula (II):

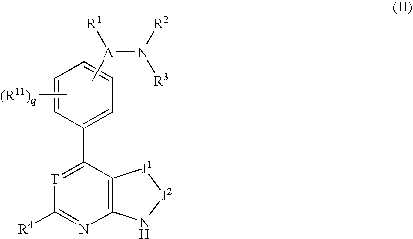

wherein the group A is attached to the meta or para position of the benzene ring, q is 0-4; T, J¹-J², A, R¹, R², R³ and R⁴ are as defined herein in respect of formula (I) and sub-groups, examples and preferences thereof; and R¹¹ is a substituent group as hereinbefore defined. In formula (II), q is preferably 0, 1 or 2, more preferably 0 or 1 and most preferably 0.

Within formula (II), the portion R¹-A-NR²R³ of the compound can be represented by the formula R¹—(CH₂)ₓ—X′—(CH₂)_{y}—NR²R³ wherein x is 0, 1 or 2, y is 0, 1 or 2 provided that the sum of x and y does not exceed 4; X¹ is attached to the group E and is a group C(R^{x}) where (i) R^{x} is hydrogen or (ii) R^{x} together with R² constitutes an alkylene linking chain of up to 3 carbon atoms in length such that the moiety X′—(CH₂)_{y}—NR²R³ forms a 4 to 7 membered saturated heterocyclic ring.

For example, one sub-group of the compounds of the formula (II) can be represented by the formula (IIa):

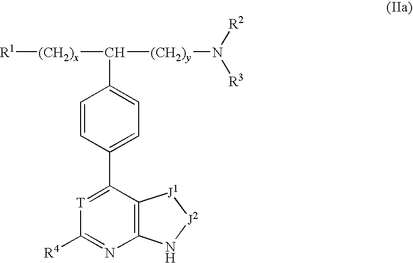

In formula (IIa), x is preferably 0 or 1 and y is 0, 1 or 2. In one embodiment, both x and y are 1. In another embodiment, x is 0 and y is 1.

Another sub-group of compounds within formula (II) can be represented by the formula (IIb):

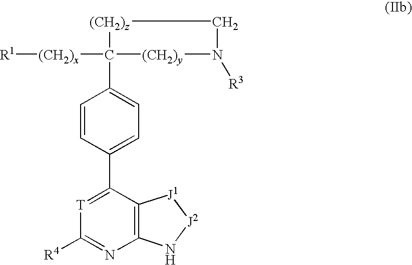

wherein R⁴, J¹-J², T, x and y are as hereinbefore defined and z is 0, 1 or 2 provided that the sum of y and z does not exceed 4. In one particular embodiment, y is 2 and z is 1.

In each of formulae (II), (IIa) and (IIb), and embodiments thereof, the group R¹ is preferably an optionally substituted aryl or heteroaryl group, and typically a monocyclic aryl or heteroaryl group of 5 or 6 ring members. Particular aryl and heteroaryl groups are phenyl, pyridyl, furanyl and thienyl groups, each optionally substituted as defined herein. Optionally substituted phenyl groups are particularly preferred.

Particular sub-groups of compounds in each of formulae (II), (IIa) and (IIb) consist of compounds in which R¹ is unsubstituted phenyl or, more preferably, phenyl bearing 1 to 3 (and more preferably 1 or 2) substituents selected from hydroxy; C₁₋₄ acyloxy; fluorine; chlorine; bromine; trifluoromethyl; cyano; C₁₋₄ hydrocarbyloxy and C₁₋₄ hydrocarbyl groups wherein the C₁₋₄ hydrocarbyloxy and C₁₋₄ hydrocarbyl groups are each optionally substituted by one or more C₁₋₂ alkoxy, halogen, hydroxy or optionally substituted phenyl or pyridyl groups; C₁₋₄ acylamino; benzoylamino; pyrrolidinocarbonyl; piperidinocarbonyl; morpholinocarbonyl; piperazinocarbonyl; five and six membered heteroaryl groups containing one or two heteroatoms selected from N, O and S. the heteroaryl groups being optionally substituted by one or more C₁₋₄ alkyl substituents; optionally substituted phenyl; optionally substituted pyridyl; and optionally substituted phenoxy; wherein the optional substituent for the phenyl, pyridyl and phenoxy groups are 1, 2 or 3 substituents selected from C₁₋₂ acyloxy, fluorine, chlorine, bromine, trifluoromethyl, cyano, and C₁₋₂ hydrocarbyloxy and C₁₋₂ hydrocarbyl groups wherein the C₁₋₂ hydrocarbyloxy and C₁₋₂ hydrocarbyl groups are each optionally substituted by methoxy or hydroxy.

More particular sub-groups of compounds within each of formulae (II), (IIa) and (IIb) consist of compounds wherein R¹ is unsubstituted phenyl or, more preferably, phenyl bearing 1 to 3 (and more preferably 1 or 2) substituents independently selected from hydroxy; C₁₋₄ acyloxy; fluorine; chlorine; bromine; trifluoromethyl; cyano; C₁₋₄ alkoxy or C₁₋₄ alkyl groups wherein the C₁₋₄ alkoxy and C₁₋₄ alkyl groups are each optionally substituted by one or more fluorine atoms or by C₁₋₂ alkoxy, hydroxy or optionally substituted phenyl; C₁₋₄ acylamino; benzoylamino; pyrrolidinocarbonyl; piperidinocarbonyl; morpholinocarbonyl; piperazinocarbonyl; optionally substituted phenyl; optionally substituted pyridyl; and optionally substituted phenoxy wherein the optionally substituted phenyl, pyridyl and phenoxy groups are each optionally substituted with 1, 2 or 3 substituents selected from C₁₋₂ acyloxy, fluorine, chlorine, bromine, trifluoromethyl, cyano, C₁₋₂ hydrocarbyloxy and C₁₋₂ hydrocarbyl each optionally substituted by methoxy or hydroxy.

Although up to 5 substituents may be present, more typically there are 0, 1, 2, 3 or 4 substituents, preferably 0, 1, 2 or 3, and more preferably 0, 1 or 2.

In one embodiment within each of formulae (II), (IIa) and (IIb), R¹ is unsubstituted phenyl or a phenyl group substituted by 1 or 2 substituents independently selected from hydroxy; C₁₋₄ acyloxy; fluorine; chlorine; bromine; trifluoromethyl; trifluoromethoxy; difluoromethoxy; benzyloxy; cyano; C₁₋₄ hydrocarbyloxy and C₁₋₄ hydrocarbyl each optionally substituted by C₁₋₂ alkoxy or hydroxy.

More preferably, the group R¹ is a substituted phenyl group bearing 1 or 2 substituents independently selected from fluorine; chlorine; trifluoromethyl; trifluoromethoxy; difluoromethoxy; cyano; methoxy, ethoxy, i-propoxy, methyl, ethyl, propyl, isopropyl, tert-butyl and benzyloxy.

In one sub-group of compounds within each of formulae (II), (IIa) and (IIb), the group R¹ is a phenyl group having a substituent at the para position selected from fluorine, chlorine, trifluoromethyl, trifluoromethoxy, difluoromethoxy, benzyloxy, methyl, tert-butyl and methoxy, and optionally a second substituent at the ortho- or meta-position selected from fluorine, chlorine or methyl. Within this sub-group, the phenyl group can be monosubstituted. Alternatively, the phenyl group can be disubstituted.

In one embodiment within each of formulae (II), (IIa) and (IIb), R¹ is selected from 4-fluorophenyl, 4-chlorophenyl and phenyl.

In a particular sub-group of compounds within each of formulae (II), (IIa) and (IIb), the group R¹ is a monosubstituted phenyl group having a chlorine substituent at the para position.

In each of formulae (II), (IIa) and (IIb) and the above embodiments, sub-groups and examples thereof:
T is preferably N; and/or
R⁴ is hydrogen; and/or
J¹-J² represents a group selected from N═CH, HN—C(O), (Me)NC(O), (Et)NC(O) and HC═CH.
   Another sub-group of compounds of the formula (I) has the general formula (III):

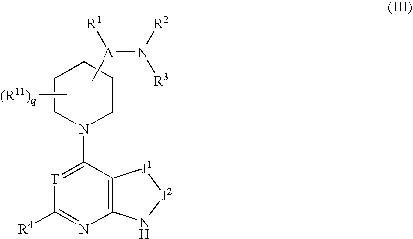

wherein the group A is attached to the 3-position or 4-position of the piperidine ring, q is 0-4; T, J¹-J², A, R¹, R², R³ and R⁴ are as defined herein in respect of formula (I) and sub-groups, examples and preferences thereof; and R¹¹ is a substituent group as hereinbefore defined. In formula (III), q is preferably 0, 1 or 2, more preferably 0 or 1 and most preferably 0.

The group R¹ is hydrogen or an aryl or heteroaryl group, wherein the aryl or heteroaryl group may be selected from the list of such groups set out in the section headed General Preferences and Definitions.

In one sub-group of compounds, R¹ is hydrogen.

In another sub-group of compounds, R¹ is an aryl or heteroaryl group.

When R¹ is aryl or heteroaryl, it can be monocyclic or bicyclic and, in one particular embodiment, is monocyclic. Particular examples of monocyclic aryl and heteroaryl groups are six membered aryl and heteroaryl groups containing up to 2 nitrogen ring members, and five membered heteroaryl groups containing up to 3 heteroatom ring members selected from O, S and N.

Examples of such groups include phenyl, naphthyl, thienyl, furan, pyrimidine and pyridine, with phenyl being presently preferred.

The aryl or heteroaryl group R¹ can be unsubstituted or substituted by up to 5 substituents, and examples of substituents are those listed in group R¹⁰ (or R^{10a} or R^{10b} or R^{10c}) above. Preferred substituents include hydroxy; C₁₋₄ acyloxy; fluorine; chlorine; bromine; trifluoromethyl; cyano; C₁₋₄ hydrocarbyloxy and C₁₋₄ hydrocarbyl each optionally substituted by C₁₋₂ alkoxy or hydroxy; C₁₋₄ acylamino; benzoylamino; pyrrolidinocarbonyl; piperidinocarbonyl; morpholinocarbonyl; piperazinocarbonyl; five and six membered heteroaryl groups containing one or two heteroatoms selected from N, O and S. the heteroaryl groups being optionally substituted by one or more C₁₋₄ alkyl substituents; phenyl; pyridyl; and phenoxy wherein the phenyl, pyridyl and phenoxy groups are each optionally substituted with 1, 2 or 3 substituents selected from C₁₋₂ acyloxy, fluorine, chlorine, bromine, trifluoromethyl, cyano, C₁₋₂ hydrocarbyloxy and C₁₋₂ hydrocarbyl each optionally substituted by methoxy or hydroxy.

Although up to 5 substituents may be present, more typically there are 0, 1, 2, 3 or 4 substituents, preferably 0, 1, 2 or 3, and more preferably 0, 1 or 2.

In one embodiment, the group R¹ is unsubstituted or substituted by up to 5 substituents selected from hydroxy; C₁₋₄ acyloxy; fluorine; chlorine; bromine; trifluoromethyl; cyano; C₁₋₄ hydrocarbyloxy and C₁₋₄ hydrocarbyl each optionally substituted by C₁₋₂ alkoxy or hydroxy.

In another embodiment, the group R¹ can have one or two substituents selected from fluorine, chlorine, trifluoromethyl, methyl and methoxy. When R¹ is a phenyl group, particular examples of substituent combinations include mono-chlorophenyl and dichlorophenyl.

When R¹ is a six membered aryl or heteroaryl group, a substituent may advantageously be present at the para position on the six-membered ring. Where a substituent is present at the para position, it is preferably larger in size than a fluorine atom.

In formula (I), R⁴ is selected from hydrogen, halogen, C₁₋₅ saturated hydrocarbyl, cyano and CF₃. Preferred values for R⁴ include hydrogen and methyl.

In formula (I), R⁵ is selected from selected from hydrogen, halogen, C₁₋₅ saturated hydrocarbyl, cyano, CONH₂, CONHR⁹, CF₃, NH₂, NHCOR⁹ and NHCONHR⁹ where R⁹ is optionally substituted phenyl or benzyl.

More preferably, R⁵ is selected from selected from hydrogen, halogen, C₁₋₅ saturated hydrocarbyl, cyano, CF₃, NH₂, NHCOR⁹ and NHCONHR⁹ where R⁹ is optionally substituted phenyl or benzyl.

The group R⁹ is typically unsubstituted phenyl or benzyl, or phenyl or benzyl substituted by 1, 2 or 3 substituents selected from halogen; hydroxy; trifluoromethyl; cyano; carboxy; C₁-₄alkoxycarbonyl; C₁₋₄ acyloxy; amino; mono- or di-C₁₋₄ alkylamino; C₁₋₄ alkyl optionally substituted by halogen, hydroxy or C₁₋₂ alkoxy; C₁₋₄ alkoxy optionally substituted by halogen, hydroxy or C₁₋₂ alkoxy; phenyl, five and six membered heteroaryl groups containing up to 3 heteroatoms selected from O, N and S; and saturated carbocyclic and heterocyclic groups containing up to 2 heteroatoms selected from O, S and N.

Particular examples of the moiety R⁵ include hydrogen, fluorine, chlorine, bromine, methyl, ethyl, hydroxyethyl, methoxymethyl, cyano, CF₃, NH₂, NHCOR^{9a} and NHCONHR^{9a} where R^{9a} is phenyl or benzyl optionally substituted by hydroxy, C₁₋₄ acyloxy, fluorine, chlorine, bromine, trifluoromethyl, cyano, C₁₋₄ hydrocarbyloxy (e.g. alkoxy) and C₁₋₄ hydrocarbyl (e.g. alkyl) optionally substituted by C₁₋₂ alkoxy or hydroxy.

In another sub-group of compounds for use according to the invention, A is a saturated hydrocarbon linker group containing from 1 to 7 carbon atoms, the linker group having a maximum chain length of 5 atoms extending between R¹ and NR²R³ and a maximum chain length of 4 atoms extending between E and NR²R³, wherein one of the carbon atoms in the linker group may optionally be replaced by an oxygen or nitrogen atom; and wherein the carbon atoms of the linker group A may optionally bear one or more substituents selected from fluorine and hydroxy, provided that the hydroxy group when present is not located at a carbon atom α with respect to the NR²R³ group; and

R⁵ is selected from selected from hydrogen, C₁₋₅ saturated hydrocarbyl, cyano, CONH₂, CF₃, NH₂, NHCOR⁹ and NHCONHR⁹.

For the avoidance of doubt, it is to be understood that each general and specific preference, embodiment and example of the groups R¹ may be combined with each general and specific preference, embodiment and example of the groups R² and/or R³ and/or R⁴ and/or R⁵ and/or R⁹ and that all such combinations are embraced by this application.

The various functional groups and substituents making up the compounds of the formula (I) are typically chosen such that the molecular weight of the compound of the formula (I) does not exceed 1000. More usually, the molecular weight of the compound will be less than 750, for example less than 700, or less than 650, or less than 600, or less than 550. More preferably, the molecular weight is less than 525 and, for example, is 500 or less.

Particular compounds for use according to the invention are as illustrated in the examples below and include:
N-methyl-N′-(9H-purin-6-yl)-propane-1,3-diamine;
6-(3-methylamino-propylamino)-7,9-dihydro-purin-8-one;
1-(4-fluorophenyl)-N³-(9H-purin-6-yl)propane-1,3-diamine;
6-[3-amino-3-(4-fluorophenyl)propylamino]-7,9-dihydropurin-8-one;
1-(4-chlorophenyl)-N³-(9H-purin-6-yl)propane-1,3-diamine;
methyl-(4-(9H-purin-6-yl)benzyl)amine;
methyl-(3-(9H-purin-6-yl)benzyl)amine;
(4-(9H-purin-6-yl)phenyl)acetonitrile;
2-(4-(9H-purin-6-yl)phenyl)ethylamine;
2-(3-(9H-purin-6-yl)phenyl)ethylamine;
1-(9H-purin-6-yl)piperidine-4-carboxylic acid amide;
C-[1-(9H-purin-6-yl)piperidin-4-yl]methylamine;
6-[4-(aminophenylmethyl)piperidin-1-yl]-7,9-dihydropurin-8-one;
6-[4-(amino(4-chlorophenyl)methyl)piperidin-1-yl]-7,9-dihydropurin-8-one;
6-(4-aminomethylpiperidin-1-yl)-7,9-dihydropurin-8-one;
3-[3-(9H-purin-6-yl)-phenoxy]-propylamine;
C-[1-(1H-pyrazolo[3,4-d]pyrimidin-4-yl)-piperidin-4-yl]-methylamine;
C-[1-(7H-pyrrolo[2,3-d]pyrimidin-4-yl)-piperidin-4-yl]-methylamine;
C-phenyl-C-[4-(9H-purin-6-yl)-phenyl]-methylamine;
2-phenyl-1-[4-(9H-purin-6-yl)-phenyl]-ethylamine;
6-[4-(1-amino-2-phenylethyl)piperidin-1-yl]-7,9-dihydropurin-8-one;
6-(4-[4-(4-chlorophenyl)-piperidin-4-yl)-phenyl)-9H-purine;
4-{4-[4-(4-chloro-phenyl)-piperidin-4-yl]-phenyl}-7H-pyrrolo[2,3-d]pyrimidine;
C-phenyl-C-[1-(7H-pyrrolo[2,3-d]pyrimidin-4-yl)-piperidin-4-yl]-methylamine;
C-4-chlorophenyl-C-[1-(7H-pyrrolo[2,3-d]pyrimidin-4-yl)-piperidin-4-yl]-methylamine;
C-(4-chloro-phenyl)-C-[1-(9H-purin-6-yl)-piperidin-4-yl]-methylamine;
4-{4-[4-(4-chloro-phenyl)-piperidin-4-yl]-phenyl}-1H-pyrrolo[2,3-b]pyridine;
C-(4-chloro-phenyl)-C-[4-(9H-purin-6-yl)-phenyl]-methylamine;
C-(4-chlorophenyl)-C-[1-(1H-pyrrolo[2,3-b]pyridin-4-yl)piperidin-4-yl]methylamine;
{2-(4-chloro-phenyl)-2-[4-(1H-pyrrolo[2,3-b]pyridin-4-yl)-phenyl]-ethyl}-methyl-amine;
C-[1-(7H-pyrrolo[2,3-d]pyrimidin-4-yl)piperidin-3-yl]methylamine; and
C-(4-chlorophenyl)-C-[1-(1H-pyrrolo[2,3-b]pyridin-4-yl)piperidin-4-yl]methylamine;
   and salts, solvates, tautomers or N-oxides thereof.

### Salts, Solvates, Tautomers, Isomers, N-Oxides, Esters, Prodrugs and Isotopes

Unless otherwise specified, a reference to a particular compound also includes ionic, salt, solvate, and protected forms thereof, for example, as discussed below.

Many compounds of the formula (I) can exist in the form of salts, for example acid addition salts or, in certain cases salts of organic and inorganic bases such as carboxylate, sulphonate and phosphate salts. All such salts are within the scope of this invention, and references to compounds of the formula (I) include the salt forms of the compounds. As in the preceding sections of this application, all references to formula (I) should be taken to refer also to formulae (II) and (III) and sub-groups thereof unless the context indicates otherwise.

Salt forms may be selected and prepared according to methods described in Pharmaceutical Salts Properties, Selection, and Use, P. Heinrich Stahl (Editor), Camille G. Wermuth (Editor), ISBN: 3-90639-026-8, Hardcover, 388 pages, August 2002.

Acid addition salts may be formed with a wide variety of acids, both inorganic and organic. Examples of acid addition salts include salts formed with an acid selected from the group consisting of acetic, 2,2-dichloroacetic, adipic, alginic, ascorbic (e.g. L-ascorbic), L-aspartic, benzenesulphonic, benzoic, 4-acetamidobenzoic, butanoic, (+) camphoric, camphor-sulphonic, (+)-(1S)-camphor-10-sulphonic, capric, caproic, caprylic, cinnamic, citric, cyclamic, dodecylsulphuric, ethane-1,2-disulphonic, ethanesulphonic, 2-hydroxyethanesulphonic, formic, fumaric, galactaric, gentisic, glucoheptonic, D-gluconic, glucuronic (e.g. D-glucuronic), glutamic (e.g. L-glutamic), α-oxoglutaric, glycolic, hippuric, hydrobromic, hydrochloric, hydriodic, isethionic, lactic (e.g. (+)-L-lactic and (±)-DL-lactic), lactobionic, maleic, malic, (−)-L-malic, malonic, (±)-DL-mandelic, methanesulphonic, naphthalenesulphonic (e.g. naphthalene-2-sulphonic), naphthalene-1,5-disulphonic, 1-hydroxy-2-naphthoic, nicotinic, nitric, oleic, orotic, oxalic, palmitic, pamoic, phosphoric, propionic, L-pyroglutamic, salicylic, 4-amino-salicylic, sebacic, stearic, succinic, sulphuric, tannic, (+)-L-tartaric, thiocyanic, toluenesulphonic (e.g. p-toluenesulphonic), undecylenic and valeric acids, as well as acylated amino acids and cation exchange resins.

For example, if the compound is anionic, or has a functional group which may be anionic (e.g., —COOH may be —COO⁻), then a salt may be formed with a suitable cation. Examples of suitable inorganic cations include, but are not limited to, alkali metal ions such as Na⁺ and K⁺, alkaline earth cations such as Ca²⁺ and Mg²⁺, and other cations such as Al³⁺. Examples of suitable organic cations include, but are not limited to, ammonium ion (i.e., NH₄⁺) and substituted ammonium ions (e.g., NH₃R⁺, NH₂R₂⁺, NHR₃⁺, NR₄⁺). Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

Where the compounds of the formula (I) contain an amine function, these may form quaternary ammonium salts, for example by reaction with an alkylating agent according to methods well known to the skilled person. Such quaternary ammonium compounds are within the scope of formula (I) as defined herein.

The salt forms of the compounds for use according to the invention are typically pharmaceutically acceptable salts, and examples of pharmaceutically acceptable salts are discussed in Berge et al., 1977, “Pharmaceutically Acceptable Salts,” J. Pharm. Sci., Vol. 66, pp. 1-19. However, salts that are not pharmaceutically acceptable may also be prepared as intermediate forms which may then be converted into pharmaceutically acceptable salts. Such non-pharmaceutically acceptable salts forms, which may be useful, for example, in the purification or separation of the compounds, also find application in relation to the invention.

Compounds of the formula (I) containing an amine function may also form N-oxides. A reference herein to a compound of the formula (I) that contains an amine function also includes the N-oxide.

Where a compound contains several amine functions, one or more than one nitrogen atom may be oxidised to form an N-oxide. Particular examples of N-oxides are the N-oxides of a tertiary amine or a nitrogen atom of a nitrogen-containing heterocycle.

N-Oxides can be formed by treatment of the corresponding amine with an oxidizing agent such as hydrogen peroxide or a per-acid (e.g. a peroxycarboxylic acid), see for example Advanced Organic Chemistry, by Jerry March, 4^{th} Edition, Wiley Interscience, pages. More particularly, N-oxides can be made by the procedure of L. W. Deady (Syn. Comm. 1977, 7, 509-514) in which the amine compound is reacted with m-chloroperoxybenzoic acid (MCPBA), for example, in an inert solvent such as dichloromethane.

Compounds of the formula (I) may exist in a number of different geometric isomeric, and tautomeric forms and references to compounds of the formula (I) include all such forms. For the avoidance of doubt, where a compound can exist in one of several geometric isomeric or tautomeric forms and only one is specifically described or shown, all others are nevertheless embraced by formula (I) as defined herein.

For example, when J¹-J² is N═CR⁶, the tautomeric forms A and B are possible for the bicyclic group.

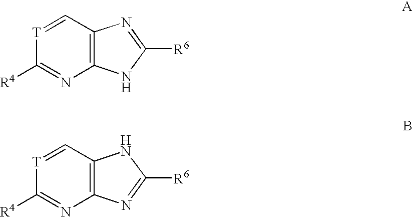

When J¹-J² is N═N, the tautomeric forms C and D are possible for the bicyclic group.

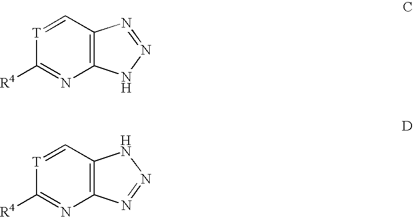

When J¹-J² is HN—CO, the tautomeric forms E, F and G are possible for the bicyclic group.

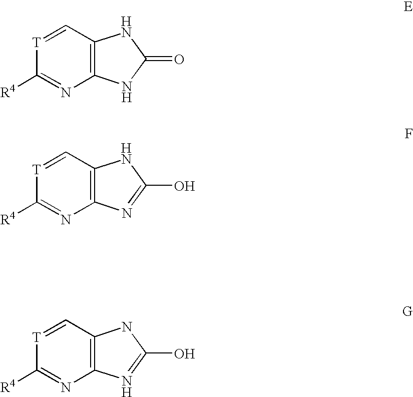

All such tautomers are embraced by formula (I) as defined herein.

Other examples of tautomeric forms include keto-, enol-, and enolate-forms, as in, for example, the following tautomeric pairs: keto/enol (illustrated below), imine/enamine, amide/imino alcohol, amidine/amidine, nitroso/oxime, thioketone/enethiol, and nitro/aci-nitro

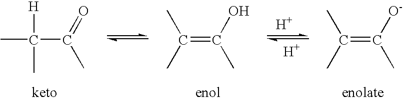

Where compounds of the formula (I) contain one or more chiral centres, and can exist in the form of two or more optical isomers, references to compounds of the formula (I) include all optical isomeric forms thereof (e.g. enantiomers, epimers and diastereoisomers), either as individual optical isomers, or mixtures (e.g. racemic mixtures) or two or more optical isomers, unless the context requires otherwise.

The optical isomers may be characterised and identified by their optical activity (i.e. as + and − isomers, or d and l isomers) or they may be characterised in terms of their absolute stereochemistry using the “R and S” nomenclature developed by Cahn, Ingold and Prelog, see Advanced Organic Chemistry by Jerry March, 4^{th} Edition, John Wiley & Sons, New York, 1992, pages 109-114, and see also Cahn, Ingold & Prelog, Angew. Chem. Int. Ed. Engl., 1966, 5, 385-415.

Optical isomers can be separated by a number of techniques including chiral chromatography (chromatography on a chiral support) and such techniques are well known to the person skilled in the art.

Where compounds of the formula (I) exist as two or more optical isomeric forms, one enantiomer in a pair of enantiomers may exhibit advantages over the other enantiomer, for example, in terms of biological activity. Thus, in certain circumstances, it may be desirable to use as a therapeutic agent only one of a pair of enantiomers, or only one of a plurality of diastereoisomers. Accordingly, the invention provides compositions containing a compound of the formula (I) having one or more chiral centres, wherein at least 55% (e.g. at least 60%, 65%, 70%, 75%, 80%, 85%, 90% or 95%) of the compound of the formula (I) is present as a single optical isomer (e.g. enantiomer or diastereoisomer). In one general embodiment, 99% or more (e.g. substantially all) of the total amount of the compound of the formula (I) may be present as a single optical isomer (e.g. enantiomer or diastereoisomer).

The compounds for use according to the invention include compounds with one or more isotopic substitutions, and a reference to a particular element includes within its scope all isotopes of the element. For example, a reference to hydrogen includes within its scope ¹H, ²H (D), and ³H (T). Similarly, references to carbon and oxygen include within their scope respectively ¹²C, ¹³C and ¹⁴C and ¹⁶O and ¹⁸O.

The isotopes may be radioactive or non-radioactive. In one embodiment of the invention, the compounds contain no radioactive isotopes. Such compounds are preferred for therapeutic use. In another embodiment, however, the compound may contain one or more radioisotopes. Compounds containing such radioisotopes may be useful in a diagnostic context.

Esters such as carboxylic acid esters and acyloxy esters of the compounds of formula (I) bearing a carboxylic acid group or a hydroxyl group are also embraced by Formula (I) as defined herein. In one embodiment of the invention, formula (I) includes within its scope esters of compounds of the formula (I) bearing a carboxylic acid group or a hydroxyl group. In another embodiment of the invention, formula (I) does not include within its scope esters of compounds of the formula (I) bearing a carboxylic acid group or a hydroxyl group. Examples of esters are compounds containing the group —C(═O)OR, wherein R is an ester substituent, for example, a C₁₋₇ alkyl group, a C₃₋₂₀ heterocyclyl group, or a C₅₋₂₀ aryl group, preferably a C₁₋₇ alkyl group. Particular examples of ester groups include, but are not limited to, —C(═O)OCH₃, —C(═O)OCH₂CH₃, —C(═O)OC(CH₃)₃, and —C(═O)OPh. Examples of acyloxy (reverse ester) groups are represented by —OC(═O)R, wherein R is an acyloxy substituent, for example, a C₁₋₇ alkyl group, a C₃₋₂₀ heterocyclyl group, or a C₅₋₂₀ aryl group, preferably a C₁₋₇ alkyl group. Particular examples of acyloxy groups include, but are not limited to, —OC(═O)CH₃ (acetoxy), —OC(═O)CH₂CH₃, —OC(═O)C(CH₃)₃, —OC(═O)Ph, and —OC(═O)CH₂Ph.

Also encompassed by formula (I) are any polymorphic forms of the compounds, solvates (e.g. hydrates), complexes (e.g. inclusion complexes or clathrates with compounds such as cyclodextrins, or complexes with metals) of the compounds, and pro-drugs of the compounds. By “prodrugs” is meant for example any compound that is converted in vivo into a biologically active compound of the formula (I) as defined herein.

For example, some prodrugs are esters of the active compound (e.g., a physiologically acceptable metabolically labile ester). During metabolism, the ester group (—C(═O)OR) is cleaved to yield the active drug. Such esters may be formed by esterification, for example, of any of the carboxylic acid groups (—C(═O)OH) in the parent compound, with, where appropriate, prior protection of any other reactive groups present in the parent compound, followed by deprotection if required.

Examples of such metabolically labile esters include those of the formula —C(═O)OR wherein R is:

C₁₋₇alkyl
(e.g., -Me, -Et, -nPr, -iPr, -nBu, -sBu, -iBu, -tBu);
C₁₋₇-aminoalkyl
(e.g., aminoethyl; 2-(N,N-diethylamino)ethyl; 2-(4-morpholino)ethyl); and
acyloxy-C₁₋₇alkyl
(e.g., acyloxymethyl;
acyloxyethyl;
pivaloyloxymethyl;
acetoxymethyl;
1-acetoxyethyl;
1-(1-methoxy-1-methyl)ethyl-carbonxyloxyethyl;
1-(benzoyloxy)ethyl; isopropoxy-carbonyloxymethyl;
1-isopropoxy-carbonyloxyethyl; cyclohexyl-carbonyloxymethyl;
1-cyclohexyl-carbonyloxyethyl;
cyclohexyloxy-carbonyloxymethyl;
1-cyclohexyloxy-carbonyloxyethyl;
(4-tetrahydropyranyloxy) carbonyloxymethyl;
1-(4-tetrahydropyranyloxy)carbonyloxyethyl;
(4-tetrahydropyranyl)carbonyloxymethyl; and
1-(4-tetrahydropyranyl)carbonyloxyethyl).

Also, some prodrugs are activated enzymatically to yield the active compound, or a compound which, upon further chemical reaction, yields the active compound (for example, as in Antibody-directed Enzyme Prodrug Therapy (ADEPT), Gene-directed Enzyme Prodrug Therapy (GDEPT), Polymer-directed Enzyme Prodrug Therapy (PDEPT), Ligand-directed Enzyme Prodrug Therapy (LIDEPT), etc.). For example, the prodrug may be a sugar derivative or other glycoside conjugate, or may be an amino acid ester derivative.

### Methods for the Preparation of Compounds of the Formula (I)

In this section, references to compounds of the formula (I) include formulae (II) and (III) and each of the sub-groups thereof as defined herein unless the context requires otherwise.

In a further aspect, the invention provides a process for the preparation of a compound of the formula (I) as defined herein.

Compounds of the formula (I) wherein E is an aryl or heteroaryl group can be prepared by reaction of a compound of the formula (X) with a compound of the formula (XI) where (X) and (XI) may be suitably protected and wherein A, E, and R¹ to R⁵ are as hereinbefore defined, one of the groups X and Y is chlorine, bromine or iodine or a trifluoromethanesulphonate (triflate) group, and the other one of the groups X and Y is a boronate residue, for example a boronate ester or boronic acid residue.

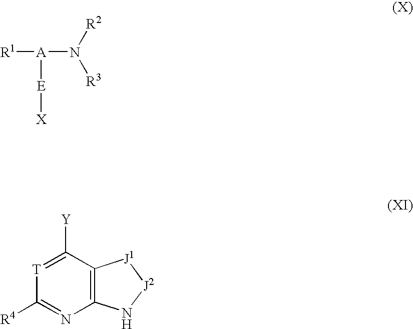

The reaction can be carried out under typical Suzuki Coupling conditions in the presence of a palladium catalyst such as tetrakis(triphenylphosphine)palladium or a palladacycle catalyst (e.g. the palladocycle catalyst described in R. B. Bedford & C.S.J. Cazin, Chem. Commun., 2001, 1540-1541) and a base (e.g. a carbonate such as potassium carbonate). The reaction may be carried out in a polar solvent, for example an aqueous solvent such as aqueous ethanol, or an ether such as dimethoxyethane or dioxane and the reaction mixture is typically subjected to heating, for example to a temperature of 80° C. or more, e.g. a temperature in excess of 100° C.

An illustrative synthetic route involving a Suzuki coupling step is shown in Scheme 1. In Scheme 1, the bromo compound (XII) in which E is an aryl or heteroaryl group, is converted to a boronic acid (XIII) by reaction with an alkyl lithium such as butyl lithium and a borate ester (iPrO)₃B. The reaction is typically carried out in a dry polar solvent such as tetrahydrofuran at a reduced temperature (for example -78° C.).

The resulting boronic acid (XIII) is then reacted with the N-protected chloro compound (XIV) in the presence of tetrakis(triphenylphosphine)palladium under the conditions described above. The protecting group PG (which can be for example a tetrahydropyranyl (THP) group) is then removed by treatment with an acid such as hydrochloric acid to give the compound of the formula (I) as defined herein.

In Scheme 1, where R² and/or R³ are hydrogen, the amino group NR²R³ is typically protecting with a suitable protecting group of which examples are set out below. One particular protecting group which may be used in the context of a Suzuki coupling is the tert-butoxycarbonyl group which can be introduced by reacting the amino group with di-tert-butylcarbonate in the presence of a base such as triethylamine. Removal of the protecting group is typically accomplished at the same time as removal of the protecting group PG on the bicyclic group.

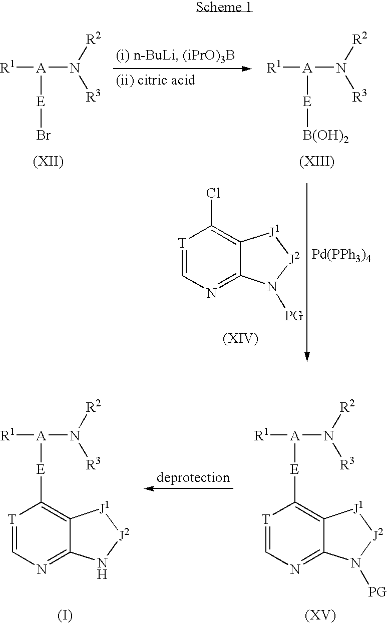

As an alternative to using a boronic acid (compound XIII) in the Suzuki coupling step, a boronate ester may be used instead. Boronate esters (for example a pinacolatoboronate) can be prepared from a compound of the formula (XII) by reaction with a diboronate ester such as bis(pinacolato)diboron in the presence of a phosphine such as tricyclohexylphosphine and a palladium (0) reagent such as tris(dibenzylideneacetone)-dipalladium (0). The formation of the boronate ester is typically carried out in a dry polar aprotic solvent such as dioxane with heating to a temperature of up to about 100° C., for example around 80° C.

Compounds of the formula (I) can also be prepared from the aldehyde compound (XVI) as shown in Scheme 2. The aldehyde compound (XVI) can be prepared by the reaction of the N-protected bicyclic chloro compound (XIV) with a boronic acid derivative of the formula (HO)₂B-E-CHO in the presence of a palladium catalyst Pd(PPh₃)₄ under the Suzuki coupling conditions described above. The aldehyde (XVI) can then be used to prepare a number of different compounds of the formula (I) as defined herein. Thus, for example, reaction of the aldehyde with tert-butyl sulphinamide in the presence of a suitable dehydrating agent, such as magnesium sulphate, and an acid catalyst, such as pyridinium p-toluenesulphonate, in dichloromethane at room temperature to give an intermediate tert-butyl sulphinylimine (not shown) followed by reaction with the Grignard reagent R¹—MgBr, where R¹ is an aryl or heteroaryl group (for example at room temperature or at reflux in tetrahydrofuran) gives the tert-butyl sulphinylamino derivative (XVII) which can then be hydrolysed and deprotected using hydrochloric acid in methanol to give the amine (XVIII).

The preparation of the corresponding compound (XIX) wherein A is CH and R¹ is hydrogen can be achieved by a reductive amination of the aldehyde (XVI) using an amine HNR²R³ and a reducing agent such as a borohydride (e.g. sodium borohydride) or a borohydride derivative (e.g. sodium cyanoborohydride or sodium triacetoxy borohydride) in a polar solvent such as ethanol or tetrahydrofuran (THF) usually at a reduced temperature.

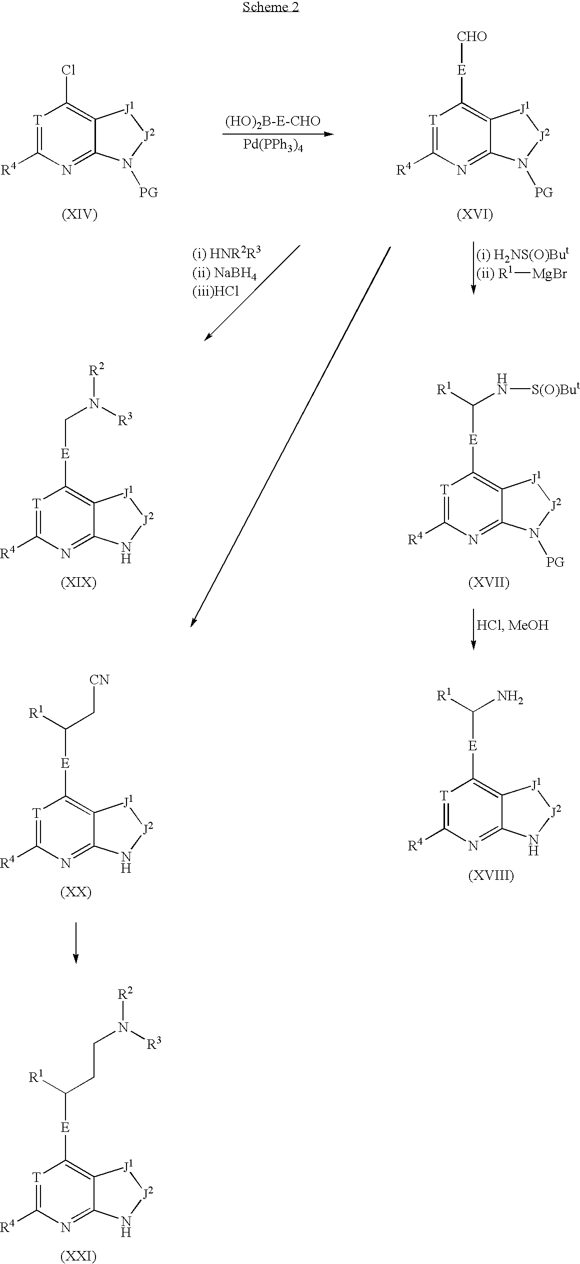

The formation of a compound of the formula (I) where ANR²R³ is CHCH₂CN or CHCH₂CH₂NR²R³ can be brought about by reacting the aldehyde (XVI) with malononitrile or ethylcyanoacetate in the presence of a base such as sodium or potassium hydroxide or an amine such a diethylamine or triethylamine under standard Knoevenagel condensation conditions (see Advanced Organic Chemistry by J. March, 4^{th} edition, John Wiley & Sons, 1992, pages 945-947 and references therein) to give an intermediate cyanoacrylate derivative (not shown). The cyanoacrylate derivative can then be reacted with a Grignard reagent R¹—MgBr and the product subjected to hydrolysis and decarboxylation to give a compound of the formula (XX) where R¹ is an aryl; or heteroaryl group. Alternatively, the cyanoacrylate derivative can be treated with a reducing agent that will selectively reduce the alkene double bond of the cyanoacrylate group without reducing the nitrile group to give the substituted acetonitrile derivative (XIV). A borohydride such as sodium borohydride may be used for this purpose The reduction reaction is typically carried out in a solvent such as ethanol and usually with heating, for example to a temperature up to about 65° C. The product is then subjected to hydrolysis and decarboxylation to give a compound of the formula (XX) where R¹ is hydrogen.

The substituted acetonitrile compound (XX) may then be reduced to the corresponding amine (XXI) by treatment with a suitable reducing agent such as Raney nickel and ammonia or hydrazine in ethanol.

Compounds of the formula (I) where A is CHCH₂ and R¹ is hydrogen may be prepared by condensing the aldehyde (XVI) with nitromethane in the presence of a base and then reducing the resulting nitroethene intermediate (not shown).

Compounds of the formula (I) wherein the group A contains a heteroatom which is attached directly to E, and E is an aryl or heteroaryl group can be formed by a process of the type illustrated in Scheme 3.

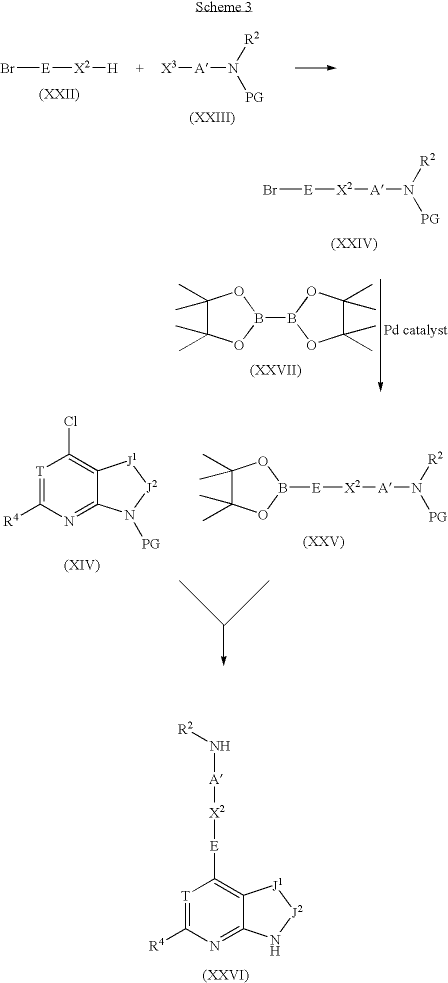

In Scheme 3, a bromoaryl or bromoheteroaryl derivative (XXII) where X² is O is reacted with a hydroxyalkyl compound (XXIII) where X³ is OH, A′ is the residue of the group A and PG is a protecting group such as a tert-butoxycarbonyl group, in a Mitsunobu coupling reaction. The Mitsunobu coupling reaction is typically carried out using diisopropylazodicarboxylate (DIAD) and triphenylphosphine as the coupling agent in a polar solvent such as THF.

Bromo compounds of the formula (XXIV) where X² is S or NH can also be formed by reacting a compound of the formula (XXII) where X² is S with a compound of the formula (XXIII) where X³ is a halogen, particularly bromine or chlorine. Compounds of the formula (XXIV) where X2 is NH can be formed by the reductive amination of a compound of the formula (XXII) where X² is NH with a compound of the formula (XXIII) where X³ is an aldehyde group.

The resulting bromo compound (XXIV) is then reacted with the diboronate reagent (XXVII) in the presence of a palladium catalyst to give the boronate derivative (XXV) which can then be coupled with the chloro-bicyclic compound (XIV) under Suzuki conditions to give, after deprotection using an acid, a compound of the formula (XXVI).

In the preparative procedures outlined above, the coupling of the aryl or heteroaryl group E to the bicyclic group is accomplished by reacting a halo-purine (or deaza analogue thereof) or halo-aryl or heteroaryl compound with a boronate ester or boronic acid in the presence of a palladium catalyst and base. Many boronates suitable for use in preparing compounds for use according to the invention are commercially available, for example from Boron Molecular Limited of Noble Park, Australia, or from Combi-Blocks Inc, of San Diego, USA. Where the boronates are not commercially available, they can be prepared by methods known in the art, for example as described in the review article by N. Miyaura and A. Suzuki, Chem. Rev. 1995, 95, 2457. Thus, boronates can be prepared by reacting the corresponding bromo-compound with an alkyl lithium such as butyl lithium and then reacting with a borate ester. The resulting boronate ester derivative can, if desired, be hydrolysed to give the corresponding boronic acid.

Compounds of the formula (I) in which the group A contains a nitrogen atom attached to the group E can be prepared by well known synthetic procedures from compounds of the formula (XXVIII) or a protected form thereof. Compounds of the formula (XXVIII) can be obtained by a Suzuki coupling reaction of a compound of the formula (XIV) (see Scheme 1) with a compound of the formula (HO)₂B-E-NH₂ or an N-protected derivative thereof.

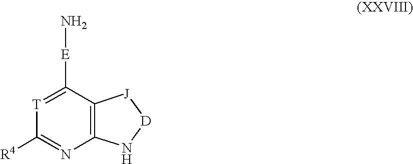

Compounds of the formula (I) wherein E is a non-aromatic cyclic group or an acyclic group and is linked to the bicyclic group by a nitrogen atom can be prepared by the reaction of a compound of the formula (XXIX) with an amine compound H₂N-G or a compound of the formula (XXX) or a protected derivative thereof, where G is as defined herein and the ring E represents a cyclic group E containing a nucleophilic NH group as a ring member.

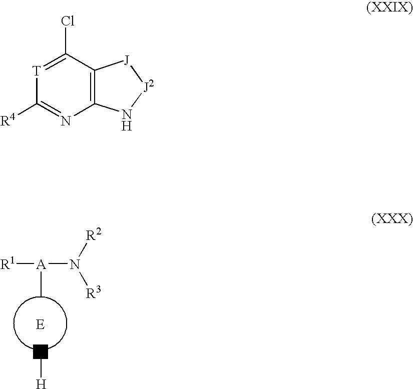

The reaction is typically carried out in a polar solvent such as an alcohol (e.g. ethanol, propanol or n-butanol) at an elevated temperature, for example a temperature in the region from 90° C. to 160° C. The reaction may be carried out in a sealed tube, particularly where the desired reaction temperature exceeds the boiling point of the solvent. When T is N, the reaction is typically carried out at a temperature in the range from about 100° C. to 130° C. but, when T is CH, higher temperatures may be required, for example up to about 160° C., and hence higher boiling solvents such as dimethylformamide may be used. In general, an excess of the nucleophilic amine will be used and/or an additional non-reacting base such as triethylamine will be included in the reaction mixture. Heating of the reaction mixture may be accomplished by normal means or by the use of a microwave heater.

In a variation on the above method, the compound of the formula (XXIX) may be reacted with a ketone of the formula (XXXI, A″ is a bond or an alkylene group such as methylene) as shown in Scheme 4.

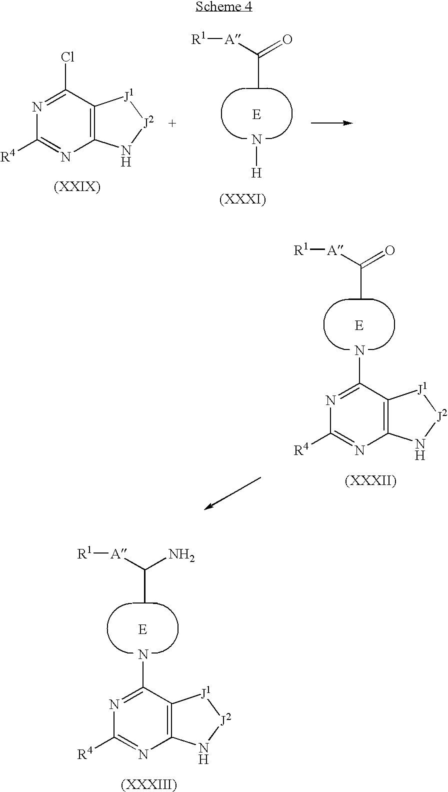

The reaction of the ketone (XXXI) with the chlorobicyclic compound (XXIX) is typically carried out in an alcoholic solvent such as n-butanol at an elevated temperature, for example in the region of 100° C. and in the presence of a non-interfering base such as triethylamine. The resulting ketone (XXXII) is then subjected to reductive amination using ammonium acetate in the presence of a reducing agent such as sodium cyanoborohydride in a polar solvent such as methanol.

Compounds of the formula (XXIX) are commercially available or can be prepared according to methods well known to the skilled person. For example, compounds of the formula (XXIX) where T is N and J¹-J² is CH═N can be prepared from the corresponding hydroxy compound by reaction with a chlorinating agent such as POCl₃. Compounds of the formula (XXIX) where J¹-J² is HN—C(O) can be prepared by the reaction of an ortho-diamino compound of the formula (XXXIV) with carbonyl di-imidazole in the presence of a non-interfering base such as triethylamine.

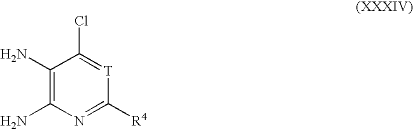

Compounds of the formula (XXIX) where T is CR⁵ and J¹-J² is (R⁷)H═CH(R⁶) can be prepared from the corresponding N-oxide of the formula (XXXV) by reaction with phosphorus oxychloride at an elevated temperature, for example the reflux temperature of POCl₃.

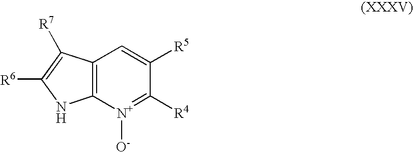

The starting materials of the formulae (X) and (XII) may be prepared by methods well known to the skilled person. For example, when E is an aryl or heteroaryl group, X is a halogen such as bromine, and the group R¹-A-NR²R³ is CH(CN)CH₂R¹, the compound of the formula (I) can be made according to the method illustrated in Scheme 5. The starting material for the synthetic route shown in Scheme 5 is the halo-substituted aryl- or heteroarylmethyl nitrile (XXXVI) in which X is a chlorine, bromine or iodine atom or a triflate group. The nitrile (XXXVI) is condensed with the aldehyde R¹CHO in the presence of an alkali such as sodium or potassium hydroxide in an aqueous solvent system such as aqueous ethanol. The reaction can be carried out at room temperature.

The resulting substituted acrylonitrile derivative (XXXVII) is then treated with a reducing agent that will selectively reduce the alkene double bond without reducing the nitrile group. A borohydride such as sodium borohydride may be used for this purpose to give the substituted acetonitrile derivative (XXXVIII). The reduction reaction is typically carried out in a solvent such as ethanol and usually with heating, for example to a temperature up to about 65° C. After reaction with a boronate compound of the formula (XI) where Y is a boronate ester or boronic acid residue under the Suzuki coupling conditions described above, the nitrile group can be reduced to the corresponding CH₂NH₂ group by treatment with a suitable reducing agent such as Raney nickel and ammonia in ethanol.

Alternatively, the nitrile group can be reduced to the amino group and an amine-protecting group introduced before coupling with the boronate.

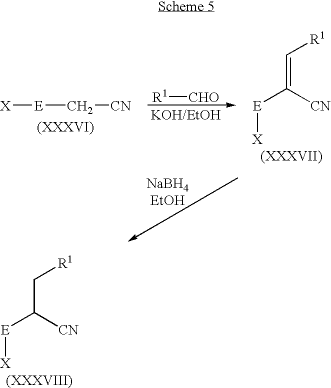

The synthetic route shown in Scheme 5 gives rise to amino compounds of the formula (X) and (XII) in which the aryl or heteroaryl group E is attached to the i-position of the group A relative to the amino group. In order to give amino compounds of the formula (X) or (XII) in which R¹ is attached to the β-position relative to the amino group, the functional groups on the two starting materials in the condensation step can be reversed so that a compound of the formula X-E-CHO wherein X is bromine, chlorine, iodine or a triflate group is condensed with a compound of the formula R¹—CH₂—CN to give a substituted acrylonitrile derivative which is then reduced to the corresponding acetonitrile derivative before coupling with the boronate (XI, Y=boronate residue) and reducing the cyano group to an amino group.

Compounds of the formula (X) or (XII) in which R¹ is attached to the α-position relative to the amino group can be prepared by the sequence of reactions shown in Scheme 6.

In Scheme 6, the starting material is a halo-substituted aryl- or heteroarylmethyl Grignard reagent (XXXIX), X=bromine or chlorine) which is reacted with the nitrile R¹—CN in a dry ether such as diethyl ether to give an intermediate imine (not shown) which is reduced to give the amine (XXXX) using a reducing agent such as lithium aluminium hydride. The amine (XXXX) can be reacted with the boronate ester or boronic acid (XI) under the Suzuki coupling conditions described above to yield a compound of the formula (I) as defined herein.

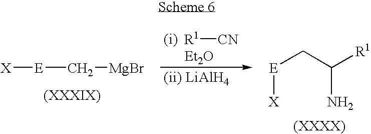

Compounds of the formula (X) and (XII) in which R¹ and E are connected to the same carbon atom can be prepared as shown in Scheme 7.

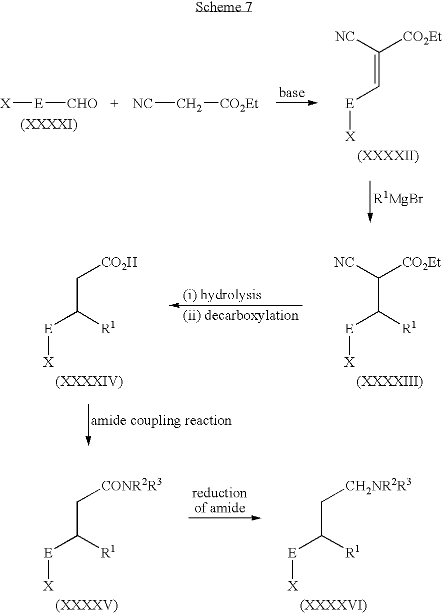

In Scheme 7, an aldehyde compound (XXXXI) where X is bromine, chlorine, iodine or a triflate group is condensed with ethyl cyanoacetate in the presence of a base to give a cyanoacrylate ester intermediate (XXXXII). The condensation is typically carried out in the presence of a base, preferably a non-hydroxide such as piperidine, by heating under Dean Stark conditions.

The cyanoacrylate intermediate (XXXXII) is then reacted with a Grignard reagent R¹MgBr suitable for introducing the group R¹ by Michael addition to the carbon-carbon double bond of the acrylate moiety. The Grignard reaction may be carried out in a polar non-protic solvent such as tetrahydrofuran at a low temperature, for example at around 0° C. The product of the Grignard reaction is the cyano propionic acid ester (XXXXIII) and this is subjected to hydrolysis and decarboxylation to give the propionic acid derivative (XXXXIV). The hydrolysis and decarboxylation steps can be effected by heating in an acidic medium, for example a mixture of sulphuric acid and acetic acid.

The propionic acid derivative (XXXXIV) is converted to the amide (XXXXV) by reaction with an amine HNR²R³ under conditions suitable for forming an amide bond. The coupling reaction between the propionic acid derivative (XXXXIV) and the amine HNR²R³ is preferably carried out in the presence of a reagent of the type commonly used in the formation of peptide linkages. Examples of such reagents include 1,3-dicyclohexylcarbodiimide (DCC) (Sheehan et al, J. Amer. Chem. Soc. 1955, 77, 1067),1-ethyl-3-(3′-dimethylaminopropyl)-carbodiimide (referred to herein either as EDC or EDAC) (Sheehan et al, J. Org. Chem., 1961, 26, 2525), uronium-based coupling agents such as O-(7-azabenzotriazol-1-yl)-N,N,N′, N′-tetramethyluronium hexafluorophosphate (HATU) and phosphonium-based coupling agents such as 1-benzo-triazolyloxytris-(pyrrolidino)phosphonium hexafluorophosphate (PyBOP) (Castro et al, Tetrahedron Letters, 1990, 31, 205). Carbodiimide-based coupling agents are advantageously used in combination with 1-hydroxy-7-azabenzotriazole (HOAt) (L. A. Carpino, J. Amer. Chem. Soc., 1993, 115, 4397) or 1-hydroxybenzotriazole (HOBt) (Konig et al, Chem. Ber., 103, 708, 2024-2034). Preferred coupling reagents include EDC (EDAC) and DCC in combination with HOAt or HOBt.

The coupling reaction is typically carried out in a non-aqueous, non-protic solvent such as acetonitrile, dioxan, dimethylsulphoxide, dichloromethane, dimethylformamide or N-methylpyrrolidine, or in an aqueous solvent optionally together with one or more miscible co-solvents. The reaction can be carried out at room temperature or, where the reactants are less reactive (for example in the case of electron-poor anilines bearing electron withdrawing groups such as sulphonamide groups) at an appropriately elevated temperature. The reaction may be carried out in the presence of a non-interfering base, for example a tertiary amine such as triethylamine or N,N-diisopropylethylamine.

Where the amine HNR²R³ is ammonia, the amide coupling reaction can be carried out using 1,1′-carbonyldiimidazole (CDI) to activate the carboxylic acid before addition of the ammonia.

As an alternative, a reactive derivative of the carboxylic acid, e.g. an anhydride or acid chloride, may be used. Reaction with a reactive derivative such an anhydride is typically accomplished by stirring the amine and anhydride at room temperature in the presence of a base such as pyridine.

The amide (XXXXV) can be converted to a compound of the formula (I) wherein A has an oxo substituent next to the NR²R³ group by reaction with the boronate (X¹) under the Suzuki coupling conditions as described above. The resulting amide of the formula (I) can subsequently be reduced using a hydride reducing agent such as lithium aluminium hydride in the presence of aluminium chloride to give a compound of the formula (I) in which NR²R³ is NH2 and wherein A is CH—CH₂—CH₂—. The reduction reaction is typically carried out in an ether solvent, for example diethyl ether, with heating to the reflux temperature of the solvent.

Rather than reacting the amide (XXXXV) with the boronate or boronic acid (XI), the amide may instead be reduced with lithium aluminium hydride/aluminium chloride, for example in an ether solvent at ambient temperature, to give the corresponding amine (XXXXVI) which may be reacted with the boronate or boronic acid (XI) under the Suzuki coupling conditions described above to give the compound of the formula (I) as defined herein.

In order to obtain the homologue of the amine containing one fewer methylene group, the carboxylic acid (XXXXIV) can be converted to the azide by standard methods and subjected to a Curtius rearrangement (see Advanced Organic Chemistry, 4^{th} edition, by Jerry March, John Wiley & sons, 1992, pages 1091-1092.

Intermediate compounds of the formula (X) where the moiety X is a chlorine, bromine or iodine atom and A is a group CH—CH₂— can be prepared by the reductive amination of an aldehyde compound of the formula (XXXXVII):

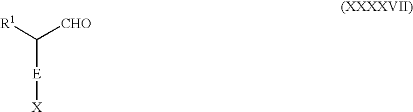

with an amine of the formula HNR²R³ under standard reductive amination conditions, for example in the presence of sodium cyanoborohydride in an alcohol solvent such as methanol or ethanol.

The aldehyde compound (XXXXVII) can be obtained by oxidation of the corresponding alcohol (XXXXVIII) using, for example, the Dess-Martin periodinane (see Dess, D. B.; Martin, J. C. J. Org. Soc., 1983, 48, 4155 and Organic Syntheses, Vol. 77, 141).

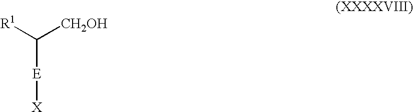

Compounds of the formula (I) where A, N and R² together form a spirocyclic group can be formed by the Suzuki coupling of a boronate or boronic acid compound of the formula (X¹) with a spirocyclic intermediate of the formula (XXXXIX) or an N-protected derivative thereof.

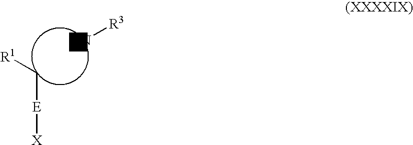

Spirocyclic intermediates of the formula (L) where R¹ is an aryl group such as an optionally substituted phenyl group, can be formed by Friedel Crafts alkylation of an aryl compound R¹—H with a compound of the formula (L):

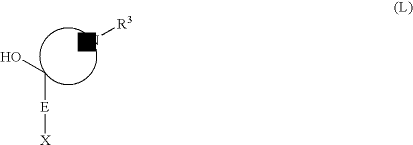

The alkylation is typically carried out in the presence of a Lewis acid such as aluminium chloride at a reduced temperature, for example less than 5° C.

In a further method for the preparation of a compound of the formula (I) wherein the moiety NR²R³ is attached to a CH₂ group of the moiety A, an aldehyde of the formula (LI) can be coupled with an amine of the formula HNR²R³ under reductive amination conditions as described above. In the formulae (LI) and (LII), A′ is the residue of the group A—i.e. the moieties A′ and CH₂ together form the group A. The aldehyde (LI) can be formed by oxidation of the corresponding alcohol (LII) using, for example, Dess-Martin periodinane.

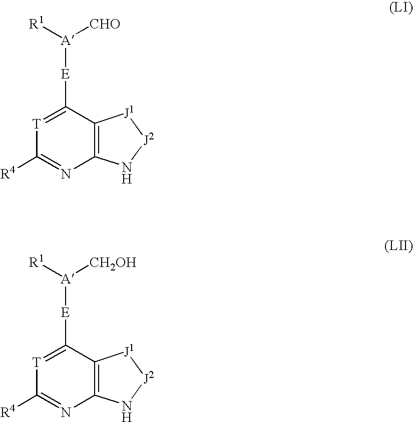

Once formed, many compounds of the formula (I) can be converted into other compounds of the formula (I) using standard functional group interconversions.

For example, compounds of the formula (I) or protected forms thereof wherein J¹-J² is CH═N can be converted into the corresponding compound where J¹-J² is N—C(CO) by bromination at the carbon atom in J¹-J² with a brominating agent such as N-bromosuccinimide (NBS) followed by hydrolysis with a mineral acid such as hydrochloric acid.

Other examples of interconversions include the reduction of compounds of the formula (I) in which the NR²R³ forms part of a nitrile group to the corresponding amine. Compounds in which NR²R³ is an NH₂ group can be converted to the corresponding alkylamine by reductive alkylation, or to a cyclic group.

Examples of functional group interconversions and reagents and conditions for carrying out such conversions can be found in, for example, Advanced Organic Chemistry, by Jerry March, 4^{th} edition, 119, Wiley Interscience, New York, Fiesers' Reagents for Organic Synthesis, Volumes 1-17, John Wiley, edited by Mary Fieser (ISBN: 0-471-58283-2), and Organic Syntheses, Volumes 1-8, John Wiley, edited by Jeremiah P. Freeman (ISBN: 0-471-31192-8).

In many of the reactions described above, it may be necessary to protect one or more groups to prevent reaction from taking place at an undesirable location on the molecule. Examples of protecting groups, and methods of protecting and deprotecting functional groups, can be found in Protective Groups in Organic Synthesis (T. Green and P. Wuts; 3rd Edition; John Wiley and Sons, 1999).

A hydroxy group may be protected, for example, as an ether (—OR) or an ester (—OC(═O)R), for example, as: a t-butyl ether; a benzyl, benzhydryl (diphenylmethyl), or trityl (triphenylmethyl)ether; a trimethylsilyl or t-butyldimethylsilyl ether; or an acetyl ester (—OC(═O)CH₃, —OAc). An aldehyde or ketone group may be protected, for example, as an acetal (R—CH(OR)₂) or ketal (R₂C(OR)₂), respectively, in which the carbonyl group (>C═O) is converted to a diether (>C(OR)₂), by reaction with, for example, a primary alcohol. The aldehyde or ketone group is readily regenerated by hydrolysis using a large excess of water in the presence of acid. An amine group may be protected, for example, as an amide (—NRCO—R) or a urethane (—NRCO—OR), for example, as: a methyl amide (—NHCO—CH₃); a benzyloxy amide (—NHCO—OCH₂C₆H₅, —NH-Cbz); as a t-butoxy amide (—NHCO—OC(CH₃)₃, —NH-Boc); a 2-biphenyl-2-propoxy amide (—NHCO—OC(CH₃)₂C₆H₄C₆H₅, —NH-Bpoc), as a 9-fluorenylmethoxy amide (—NH-Fmoc), as a 6-nitroveratryloxy amide (—NH-Nvoc), as a 2-trimethylsilylethyloxy amide (—NH-Teoc), as a 2,2,2-trichloroethyloxy amide (—NH-Troc), as an allyloxy amide (—NH-Alloc), or as a 2-(phenylsulphonyl)ethyloxy amide (—NH-Psec). Other protecting groups for amines, such as cyclic amines and heterocyclic N—H groups, include toluenesulphonyl (tosyl) and methanesulphonyl (mesyl) groups and benzyl groups such as a para-methoxybenzyl (PMB) group. A carboxylic acid group may be protected as an ester for example, as: an C₁₋₇ alkyl ester (e.g., a methyl ester; a t-butyl ester); a C₁₋₇ haloalkyl ester (e.g., a C₁₋₇ trihaloalkyl ester); a triC₁₋₇ alkylsilyl-C₁₋₇alkyl ester; or a C₅₋₂₀ aryl-C₁₋₇ alkyl ester (e.g., a benzyl ester; a nitrobenzyl ester); or as an amide, for example, as a methyl amide. A thiol group may be protected, for example, as a thioether (—SR), for example, as: a benzyl thioether; an acetamidomethyl ether (—S—CH₂NHC(═O)CH₃).

### Isolation and Purification of the Compounds for use According to the Invention

The compounds for use according to the invention can be isolated and purified according to standard techniques well known to the person skilled in the art. One technique of particular usefulness in purifying the compounds is preparative liquid chromatography using mass spectrometry as a means of detecting the purified compounds emerging from the chromatography column.

Preparative LC-MS is a standard and effective method used for the purification of small organic molecules such as the compounds described herein. The methods for the liquid chromatography (LC) and mass spectrometry (MS) can be varied to provide better separation of the crude materials and improved detection of the samples by MS. Optimisation of the preparative gradient LC method will involve varying columns, volatile eluents and modifiers, and gradients. Methods are well known in the art for optimising preparative LC-MS methods and then using them to purify compounds. Such methods are described in Rosentreter U. Huber U.; Optimal fraction collecting in preparative LC/MS; J Comb Chem.; 2004; 6(2), 159-64 and LeisterW, Strauss K, Wisnoski D, Zhao Z. Lindsley C., Development of a custom high-throughput preparative liquid chromatography/mass spectrometer platform for the preparative purification and analytical analysis of compound libraries; J Comb Chem.; 2003; 5(3); 322-9.

### Pharmaceutical Formulations

While it is possible for the compound for use according to the invention to be administered alone, it is preferable to present it as a pharmaceutical composition (e.g. formulation) comprising at least one active compound for use according to the invention together with one or more pharmaceutically acceptable carriers, adjuvants, excipients, diluents, fillers, buffers, stabilisers, preservatives, lubricants, or other materials well known to those skilled in the art and optionally other therapeutic or prophylactic agents

Thus, the present invention further provides pharmaceutical compositions, as defined above, and methods of making a pharmaceutical composition comprising admixing at least one active compound, as defined above, together with one or more pharmaceutically acceptable carriers, excipients, buffers, adjuvants, stabilizers, or other materials, as described herein.

The term “pharmaceutically acceptable” as used herein pertains to compounds, materials, compositions, and/or dosage forms which are, within the scope of sound medical judgment, suitable for use in contact with the tissues of a subject (e.g. human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation.

Pharmaceutical compositions containing compounds of the formula (I) can be formulated in accordance with known techniques, see for example, Remington's Pharmaceutical Sciences, Mack Publishing Company, Easton, Pa., USA.

Accordingly, in a further aspect, the invention provides compounds of the formula (I) and sub-groups thereof as defined herein in the form of pharmaceutical compositions.

The pharmaceutical compositions can be in any form suitable for oral, parenteral, topical, intranasal, ophthalmic, otic, rectal, intra-vaginal, or transdermal administration. Where the compositions are intended for parenteral administration, they can be formulated for intravenous, intramuscular, intraperitoneal, subcutaneous administration or for direct delivery into a target organ or tissue by injection, infusion or other means of delivery. The delivery can be by bolus injection, short term infusion or longer term infusion and can be via passive delivery or through the utilisation of a suitable infusion pump.

Pharmaceutical formulations adapted for parenteral administration include aqueous and non-aqueous sterile injection solutions which may contain anti-oxidants, buffers, bacteriostats, co-solvents, organic solvent mixtures, cyclodextrin complexation agents, emulsifying agents (for forming and stabilizing emulsion formulations), liposome components for forming liposomes, gellable polymers for forming polymeric gels, lyophilisation protectants and combinations of agents for, inter alia, stabilising the active ingredient in a soluble form and rendering the formulation isotonic with the blood of the intended recipient. Pharmaceutical formulations for parenteral administration may also take the form of aqueous and non-aqueous sterile suspensions which may include suspending agents and thickening agents (R. G. Strickly, Solubilizing Excipients in oral and injectable formulations, Pharmaceutical Research, Vol 21(2) 2004, p 201-230).

Liposomes are closed spherical vesicles composed of outer lipid bilayer membranes and an inner aqueous core and with an overall diameter of <100 μm. Depending on the level of hydrophobicity, moderately hydrophobic drugs can be solubilized by liposomes if the drug becomes encapsulated or intercalated within the liposome. Hydrophobic drugs can also be solubilized by liposomes if the drug molecule becomes an integral part of the lipid bilayer membrane, and in this case, the hydrophobic drug is dissolved in the lipid portion of the lipid bilayer.

The formulations may be presented in unit-dose or multi-dose containers, for example sealed ampoules and vials, and may be stored in a freeze-dried (lyophilised) condition requiring only the addition of the sterile liquid carrier, for example water for injections, immediately prior to use.

The pharmaceutical formulation can be prepared by lyophilising a compound of formula (I) as defined herein, or sub-groups thereof. Lyophilisation refers to the procedure of freeze-drying a composition. Freeze-drying and lyophilisation are therefore used herein as synonyms.

Extemporaneous injection solutions and suspensions may be prepared from sterile powders, granules and tablets.

Pharmaceutical compositions of the present invention for parenteral injection can also comprise pharmaceutically acceptable sterile aqueous or non-aqueous solutions, dispersions, suspensions or emulsions as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol, and the like), carboxymethylcellulose and suitable mixtures thereof, vegetable oils (such as olive oil), and injectable organic esters such as ethyl oleate. Proper fluidity can be maintained, for example, by the use of coating materials such as lecithin, by the maintenance of the required particle size in the case of dispersions, and by the use of surfactants.

The compositions of the present invention may also contain adjuvants such as preservatives, wetting agents, emulsifying agents, and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents such as sugars, sodium chloride, and the like. Prolonged absorption of the injectable pharmaceutical form may be brought about by the inclusion of agents which delay absorption such as aluminum monostearate and gelatin.

In one preferred embodiment of the invention, the pharmaceutical composition is in a form suitable for i.v. administration, for example by injection or infusion. For intravenous administration, the solution can be dosed as is, or can be injected into an infusion bag (containing a pharmaceutically acceptable excipient, such as 0.9% saline or 5% dextrose), before administration.

In another preferred embodiment, the pharmaceutical composition is in a form suitable for sub-cutaneous (s.c.) administration.

Pharmaceutical dosage forms suitable for oral administration include tablets, capsules, caplets, pills, lozenges, syrups, solutions, powders, granules, elixirs and suspensions, sublingual tablets, wafers or patches and buccal patches.

Thus, tablet compositions can contain a unit dosage of active compound together with an inert diluent or carrier such as a sugar or sugar alcohol, e.g.; lactose, sucrose, sorbitol or mannitol; and/or a non-sugar derived diluent such as sodium carbonate, calcium phosphate, calcium carbonate, or a cellulose or derivative thereof such as methyl cellulose, ethyl cellulose, hydroxypropyl methyl cellulose, and starches such as corn starch. Tablets may also contain such standard ingredients as binding and granulating agents such as polyvinylpyrrolidone, disintegrants (e.g. swellable crosslinked polymers such as crosslinked carboxymethylcellulose), lubricating agents (e.g. stearates), preservatives (e.g. parabens), antioxidants (e.g. BHT), buffering agents (for example phosphate or citrate buffers), and effervescent agents such as citrate/bicarbonate mixtures. Such excipients are well known and do not need to be discussed in detail here.

Capsule formulations may be of the hard gelatin or soft gelatin variety and can contain the active component in solid, semi-solid, or liquid form. Gelatin capsules can be formed from animal gelatin or synthetic or plant derived equivalents thereof.

The solid dosage forms (e.g.; tablets, capsules etc.) can be coated or un-coated, but typically have a coating, for example a protective film coating (e.g. a wax or varnish) or a release controlling coating. The coating (e.g. a Eudragit™ type polymer) can be designed to release the active component at a desired location within the gastrointestinal tract. Thus, the coating can be selected so as to degrade under certain pH conditions within the gastrointestinal tract, thereby selectively release the compound in the stomach or in the ileum or duodenum.

Instead of, or in addition to, a coating, the drug can be presented in a solid matrix comprising a release controlling agent, for example a release delaying agent which may be adapted to selectively release the compound under conditions of varying acidity or alkalinity in the gastrointestinal tract. Alternatively, the matrix material or release retarding coating can take the form of an erodible polymer (e.g. a maleic anhydride polymer) which is substantially continuously eroded as the dosage form passes through the gastrointestinal tract. As a further alternative, the active compound can be formulated in a delivery system that provides osmotic control of the release of the compound. Osmotic release and other delayed release or sustained release formulations may be prepared in accordance with methods well known to those skilled in the art.

The pharmaceutical compositions comprise from approximately 1% to approximately 95%, preferably from approximately 20% to approximately 90%, active ingredient. Pharmaceutical compositions according to the invention may be, for example, in unit dose form, such as in the form of ampoules, vials, suppositories, dragees, tablets or capsules.

Pharmaceutical compositions for oral administration can be obtained by combining the active ingredient with solid carriers, if desired granulating a resulting mixture, and processing the mixture, if desired or necessary, after the addition of appropriate excipients, into tablets, dragee cores or capsules. It is also possible for them to be incorporated into plastics carriers that allow the active ingredients to diffuse or be released in measured amounts.

The compounds for use according to the invention can also be formulated as solid dispersions. Solid dispersions are homogeneous extremely fine disperse phases of two or more solids. Solid solutions (molecularly disperse systems), one type of solid dispersion, are well known for use in pharmaceutical technology (see (Chiou and Riegelman, J. Pharm. Sci., 60, 1281-1300 (1971)) and are useful in increasing dissolution rates and increasing the bioavailability of poorly water-soluble drugs.

This invention also provides solid dosage forms comprising the solid solution described above. Solid dosage forms include tablets, capsules and chewable tablets. Known excipients can be blended with the solid solution to provide the desired dosage form. For example, a capsule can contain the solid solution blended with (a) a disintegrant and a lubricant, or (b) a disintegrant, a lubricant and a surfactant. A tablet can contain the solid solution blended with at least one disintegrant, a lubricant, a surfactant, and a glidant. The chewable tablet can contain the solid solution blended with a bulking agent, a lubricant, and if desired an additional sweetening agent (such as an artificial sweetener), and suitable flavours.

The pharmaceutical formulations may be presented to a patient in “patient packs” containing an entire course of treatment in a single package, usually a blister pack. Patient packs have an advantage over traditional prescriptions, where a pharmacist divides a patient's supply of a pharmaceutical from a bulk supply, in that the patient always has access to the package insert contained in the patient pack, normally missing in patient prescriptions. The inclusion of a package insert has been shown to improve patient compliance with the physician's instructions.

Compositions for topical use include ointments, creams, sprays, patches, gels, liquid drops and inserts (for example intraocular inserts). Such compositions can be formulated in accordance with known methods.

Examples of formulations for rectal or intra-vaginal administration include pessaries and suppositories which may be, for example, formed from a shaped moldable or waxy material containing the active compound.

Compositions for administration by inhalation may take the form of inhalable powder compositions or liquid or powder sprays, and can be administrated in standard form using powder inhaler devices or aerosol dispensing devices. Such devices are well known. For administration by inhalation, the powdered formulations typically comprise the active compound together with an inert solid powdered diluent such as lactose.

The compounds of the formula (I) will generally be presented in unit dosage form and, as such, will typically contain sufficient compound to provide a desired level of biological activity. For example, a formulation may contain from 1 nanogram to 2 grams of active ingredient, e.g. from 1 nanogram to 2 milligrams of active ingredient. Within this range, particular sub-ranges of compound are 0.1 milligrams to 2 grams of active ingredient (more usually from 10 milligrams to 1 gram, e.g. 50 milligrams to 500 milligrams), or 1 microgram to 20 milligrams (for example 1 microgram to 10 milligrams, e.g. 0.1 milligrams to 2 milligrams of active ingredient).

For oral compositions, a unit dosage form may contain from 1 milligram to 2 grams, more typically 10 milligrams to 1 gram, for example 50 milligrams to 1 gram, e.g. 100 milligrams to 1 gram, of active compound.

The active compound will be administered to a patient in need thereof (for example a human or animal patient) in an amount sufficient to achieve the desired therapeutic effect.

### Therapeutic Uses

The compounds of formula (I) modulate (e.g. inhibit) the activity of ROCK kinase or protein kinase p70S6K. The compounds therefore find application in: (a) the treatment or prophylaxis of a disease or condition in which the modulation (e.g. inhibition) of ROCK kinase or protein kinase p70S6K is indicated; and/or (b) the treatment of a subject or patient population in which the modulation (e.g. inhibition) of ROCK kinase or protein kinase p70S6K is indicated; and/or (c) the treatment or prophylaxis of a disease or condition in which the modulation (e.g. inhibition) of the Rho signalling pathway is indicated; and/or (d) the treatment of a subject or patient population in which the modulation (e.g. inhibition) of the Rho signalling pathway is indicated.

### Applicable Diseases and Conditions Related to ROCK Kinase Modulation

The invention therefore finds application in relation to diseases and conditions selected from: (a) tumour metastasis; (b) tumour invasion; (c) tumour progression; (d) tumour adhesion (e.g. tumour cell adhesion); (e) actinomycin contractility-dependent tumour metastasis, invasion or progression; (f) cell transformation; (g) ROCK-mediated tumour metastasis, invasion, progression or adhesion; (h) ROCK-mediated actinomycin contractility-dependent tumour metastasis, invasion or progression; (i) ROCK-mediated cell transformation.

The invention also finds application in relation to cancer (e.g. ROCK-mediated cancer), especially where the cancer (for example being a ROCK-mediated cancer) is selected from: (a) testicular germ cell tumours; (b) small breast carcinomas with metastatic ability; (c) bladder cancer; (d) ovarian cancer; (e) prostate cancer; and (f) hepatocellular carcinoma.

Other applicable diseases and conditions include the invasion, metastasis and tumour progression of any of the cancers defined herein.

The invention also finds application in relation to cardiovascular diseases or conditions, particularly those selected from: (a) hypertension; (b) heart dysfunction (e.g. chronic and congestive heart failure); (c) cardiac hypertrophy; (d) restenosis; (e) renal dysfunction (e.g. chronic renal failure); (f) atherosclerosis (arteriosclerosis); (g) cardioprotection; (h) allograft survival; (i) cerebral ischemia; (j) coronary vasospasm; and (k) vascular inflammation.

Other applicable diseases and conditions include muscle (e.g. smooth muscle) dysfunction, for example selected from: (a) asthma; (b) penile erectile dysfunction; (c) female sexual dysfunction; (d) over-active bladder I syndrome; and (e) abnormal smooth muscle (e.g. associated with hypertension).

Other applicable diseases and conditions include inflammation, wherein for example the inflammation comprises or is manifest by: (a) rheumatoid arthritis; (b) irritable bowel syndrome; (c) inflammatory bowel disease; (d) vascular inflammation, and (e) a neuroinflammatory disease or condition.

In embodiments relating to neuroinflammatory diseases or conditions, these may be selected from: (a) stroke; (b) multiple sclerosis; (c) Alzheimer's disease; (d) Parkinson's disease; (e) amyotrophic lateral sclerosis; and (f) inflammatory pain.

Other applicable diseases and conditions include CNS diseases or conditions, including those selected from: (a) spinal cord injury or trauma; (b) brain injury or trauma; (c) acute neuronal injury (e.g. stroke or traumatic brain injury); (d) Parkinson's disease; (e) Alzheimer's disease; (f) neurodegenerative conditions or diseases; (g) stroke (e.g. associated with hypertension); (h) cerebral vasospasm; (i) inhibition of neurite growth and sprouting; (j) inhibited neurite regeneration; (k) compromised post-trauma functional recovery; (l) demyelinating diseases or disorders; (m) inflammatory CNS diseases or disorders; (n) neuropathic pain; and (O) neurodegeneration.

Other applicable CNS diseases or conditions include those selected from: Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer Disease or dementia including dementia of mixed vascular and degenerative origin, pre-senile dementia, senile dementia and dementia associated with Parkinson's Disease, progressive supranuclear palsy or cortical basal degeneration, Parkinson's Disease, Frontotemporal dementia Parkinson's Type, Parkinson dementia complex of Guam, HIV dementia, diseases with associated neurofibrillar tangle pathologies, dementia pugilistica, amyotrophic lateral sclerosis, corticobasal degeneration, Down syndrome, Huntington's Disease, postencephelatic parkinsonism, progressive supranuclear palsy, Pick's Disease, Niemann-Pick's Disease, stroke, head trauma and other chronic neurodegenerative diseases, Bipolar Disease, affective disorders, depression, anxiety, schizophrenia, cognitive disorders, hair loss, contraceptive medication, predemented states, Age-Associated Memory Impairment, Age-Related Cognitive Decline, Cognitive Impairement No Dementia, mild cognitive decline, mild neurocognitive decline, Late-Life Forgetfulness, memory impairment and cognitive impairment, vascular dementia, dementia with Lewy bodies, Frontotemporal dementia and androgenetic alopecia.

Yet other applicable diseases and conditions include: (a) insulin resistance; (b) graft protection (e.g. cardiovascular or inflammatory graft protection); (c) diabetes; (d) asthma; (e) pulmonary vasoconstriction; (f) glaucoma; and (g) fibroses (e.g. liver fibrosis and kidney fibrosis).

Other applicable diseases and conditions include infectious diseases or conditions, including metazoan, protozoan, fungal, prion, viral or bacterial infestations, diseases or infections.

In such embodiments, the infectious disease or condition may comprise pathogen-mediated cytoskeletal rearrangement.

Proliferative Disorders (including cancers): The invention also finds application as a means of preventing the growth of or inducing apoptosis of neoplasias. The invention will therefore prove useful in treating or preventing proliferative disorders such as cancers. Examples of such abnormalities include but are not limited to overexpression of one or more of the Rho signalling pathway members, or mutations in said members which lead to an increase in the basal activity of ROCK kinas(s) or the Rho signalling pathway (which may for example be associated with upregulation or overexpression or mutational activation of a growth factor receptor such as a growth factor selected from the epidermal growth factor receptor (EGFR), fibroblast growth factor receptor (FGFR), platelet derived growth factor receptor (PDGFR), insulin-like growth factor 1 receptor (IGF-1R) and vascular endothelial growth factor receptor (VEGFR) families).

The invention will be useful in treating other conditions which result from disorders in proliferation or survival such as viral infections, and neurodegenerative diseases for example.

The invention therefore finds broad application in the treatment of diseases in which there is a disorder of proliferation, apoptosis or differentiation.

Examples of cancers which may be inhibited include, but are not limited to, a carcinoma, for example a carcinoma of the bladder, breast, colon (e.g. colorectal carcinomas such as colon adenocarcinoma and colon adenoma), kidney, epidermal, liver, lung, for example adenocarcinoma, small cell lung cancer and non-small cell lung carcinomas, oesophagus, gall bladder, ovary, pancreas e.g. exocrine pancreatic carcinoma, stomach, cervix, endometrium, thyroid, prostate, or skin, for example squamous cell carcinoma; a hematopoetic malignancy for example acute myeloid leukaemia, acute promyelocytic leukaemia, acute lymphoblastic leukaemia, chronic myeloid leukaemia, chronic lymphocytic leukaemia and other B-cell lymphoproliferative diseases, myelodysplastic syndrome, T-cell lymphoproliferative diseases including those derived from Natural Killer cells, Non-Hodgkin's lymphoma and Hodgkin's disease; Bortezomib sensitive and refractory multiple myeloma; hematopoetic diseases of abnormal cell proliferation whether pre malignant or stable such as myeloproliferative diseases including polycythemia vera, essential thrombocythemia and primary myelofibrosis; hairy cell lymphoma or Burkett's lymphoma; a hematopoietic tumour of myeloid lineage, for example acute and chronic myelogenous leukaemias, myelodysplastic syndrome, or promyelocytic leukaemia; thyroid follicular cancer; a tumour of mesenchymal origin, for example fibrosarcoma or habdomyosarcoma; a tumour of the central or peripheral nervous system, for example astrocytoma, neuroblastoma, glioma or schwannoma; melanoma; seminoma; teratocarcinoma; osteosarcoma; xenoderoma pigmentosum; keratoctanthoma; thyroid follicular cancer; or Kaposi's sarcoma.

Particular subsets of cancers include breast cancer, ovarian cancer, colon cancer, prostate cancer, oesophageal cancer, squamous cancer and non-small cell lung carcinomas. A further subset of cancers includes breast cancer, ovarian cancer, prostate cancer, endometrial cancer and glioma.

Immune Disorders Immune disorders for which the invention may be beneficial include but are not limited to autoimmune conditions and chronic inflammatory diseases, for example systemic lupus erythematosus, autoimmune mediated glomerulonephritis, rheumatoid arthritis, psoriasis, inflammatory bowel disease, and autoimmune diabetes mellitus, Eczema hypersensitivity reactions, asthma, COPD, rhinitis, and upper respiratory tract disease.

Other Therapeutic Uses: ROCK-mediated physiological processes play a role in apoptosis, proliferation, differentiation and therefore the invention could also be useful in the treatment of the following diseases other than cancer and those associated with immune dysfunction; viral infections, for example herpes virus, pox virus, Epstein-Barr virus, Sindbis virus, adenovirus, HIV, HPV, HCV and HCMV; prevention of AIDS development in HIV-infected individuals; cardiovascular diseases for example cardiac hypertrophy, restenosis, atherosclerosis; neurodegenerative disorders, for example Alzheimer's disease, AIDS-related dementia, Parkinson's disease, amyotropic lateral sclerosis, retinitis pigmentosa, spinal muscular atropy and cerebellar degeneration; glomerulonephritis; myelodysplastic syndromes, ischemic injury associated myocardial infarctions, stroke and reperfusion injury, degenerative diseases of the musculoskeletal system, for example, osteoporosis and arthritis, aspirin-sensitive rhinosinusitis, cystic fibrosis, multiple sclerosis, kidney diseases.

The invention may also be useful in diseases resulting from insulin resistance and insensitivity, and the disruption of glucose, energy and fat storage such as metabolic disease and obesity.

### Applicable Diseases and Conditions Related to Protein Kinase P70S6K Modulation

The invention therefore finds application in relation to conditions selected from: (a) cancer (e.g. p70S6K-mediated cancer); (b) tumour metastases; (c) immune dysfunction; (d) tissue damage (e.g. arising from inflammation); (e) chromosome 17q23 amplification (or conditions arising therefrom or associated therewith); (f) Peutz-Jeghers syndrome (or conditions arising therefrom or associated therewith); (g) LKB1 mutation(s) (or conditions arising therefrom or associated therewith); (h) BRCA1 mutation(s) (or conditions arising therefrom or associated therewith); (i) BRCA2 mutation(s) (or conditions arising therefrom or associated therewith); (j) dysfunctional apoptotic programmes; (k) growth factor receptor signal transduction, overexpression and activation in tumour tissue; (l) a metabolic disease or disorder; (m) those associated with abnormal cell proliferation and/or metabolism; and (n) neuronal disorders.

In such embodiments, the disease or condition arising from or associated with chromosome 17q23 amplification may be selected from: (a) primary breast tumours; (b) tumours (e.g. breast tumours) containing BRCA2 mutations; (c) tumours (e.g. breast tumours) containing BRCA1 mutations; (d) pancreatic tumours; (e) bladder tumours; and (f) neuroblastomas.

The disease or condition arising from or associated with LKB1 mutation(s) may be lung adenocarcinoma containing LKB1 mutation(s) (e.g. inactivating LKB1 mutation(s)).

The disease or condition arising from or associated with BRCA1/2 mutation(s) may be breast cancer.

The metabolic disease or disorder may be selected from: (a) obesity (for example age-induced obesity or diet-induced obesity); (b) diabetes; (c) metabolic syndrome; (d) insulin resistance; (e) hyperglycemia; (f) hyperaminoacidemia; and (g) hyperlipidmia.

Proliferative Disorders (including cancers): The invention also finds application as a means of preventing the growth of or inducing apoptosis of neoplasias. The invention will therefore prove useful in treating or preventing proliferative disorders such as cancers. Examples of such abnormalities include but are not limited to overexpression of p70S6K (or the other syndromes described herein).

The invention will be useful in treating other conditions which result from disorders in proliferation or survival such as viral infections, and neurodegenerative diseases for example.

The invention therefore finds broad application in the treatment of diseases in which there is a disorder of proliferation, apoptosis or differentiation.

Examples of cancers which may be inhibited include, but are not limited to, a carcinoma, for example a carcinoma of the bladder, breast, colon (e.g. colorectal carcinomas such as colon adenocarcinoma and colon adenoma), kidney, epidermal, liver, lung, for example adenocarcinoma, small cell lung cancer and non-small cell lung carcinomas, oesophagus, gall bladder, ovary, pancreas e.g. exocrine pancreatic carcinoma, stomach, cervix, endometrium, thyroid, prostate, or skin, for example squamous cell carcinoma; a hematopoietic tumour of lymphoid lineage, for example leukaemia, acute lymphocytic leukaemia, B-cell lymphoma, T-cell lymphoma, Hodgkin's lymphoma, non-Hodgkin's lymphoma, hairy cell lymphoma, or Burkett's lymphoma; a hematopoietic tumour of myeloid lineage, for example acute and chronic myelogenous leukaemias, myelodysplastic syndrome, or promyelocytic leukaemia; thyroid follicular cancer; a tumour of mesenchymal origin, for example fibrosarcoma or habdomyosarcoma; a tumour of the central or peripheral nervous system, for example astrocytoma, neuroblastoma, glioma or schwannoma; melanoma; seminoma; teratocarcinoma; osteosarcoma; xenoderoma pigmentosum; keratoctanthoma; thyroid follicular cancer; or Kaposi's sarcoma.

Particular subsets of cancers include breast cancer, ovarian cancer, colon cancer, prostate cancer, oesophageal cancer, squamous cancer and non-small cell lung carcinomas. A further subset of cancers includes breast cancer, ovarian cancer, prostate cancer, endometrial cancer and glioma.

Immune Disorders: Immune disorders for which the invention may be beneficial include but are not limited to autoimmune conditions and chronic inflammatory diseases, for example systemic lupus erythematosus, autoimmune mediated glomerulonephritis, rheumatoid arthritis, psoriasis, inflammatory bowel disease, and autoimmune diabetes mellitus, Eczema hypersensitivity reactions, asthma, COPD, rhinitis, and upper respiratory tract disease.

Other Therapeutic Uses: p70S6K -mediated physiological processes play a role in apoptosis, proliferation, differentiation and therefore the invention could also be useful in the treatment of the following diseases other than cancer and those associated with immune dysfunction; viral infections, for example herpes virus, pox virus, Epstein-Barr virus, Sindbis virus, adenovirus, HIV, HPV, HCV and HCMV; prevention of AIDS development in HIV-infected individuals; cardiovascular diseases for example cardiac hypertrophy, restenosis, atherosclerosis; neurodegenerative disorders, for example Alzheimer's disease, AIDS-related dementia, Parkinson's disease, amyotropic lateral sclerosis, retinitis pigmentosa, spinal muscular atropy and cerebellar degeneration; glomerulonephritis; myelodysplastic syndromes, ischemic injury associated myocardial infarctions, stroke and reperfusion injury, degenerative diseases of the musculoskeletal system, for example, osteoporosis and arthritis, aspirin-sensitive rhinosinusitis, cystic fibrosis, multiple sclerosis, kidney diseases.

The invention may also be useful in diseases resulting from insulin resistance and insensitivity, and the disruption of glucose, energy and fat storage such as metabolic disease and obesity.

### Applicable Interventions, Treatments and Prophylactic Methods Related to ROCK Kinase Modulation

The invention contemplates ROCK-mediated intervention, treatment or prophylaxis of any kind. Thus, the invention finds application in relation to treatment or prophylaxis comprising: (a) the modulation (e.g. inhibition) of ROCK kinase; or (b) intervention at the level of the activity of ROCK kinase; or (c) intervention at the level of the Rho signalling pathway (e.g. at the level of RhoA and or RhoC).

Other applicable methods include interventions which effect: (a) muscle (e.g. smooth muscle) relaxation; (b) vascular muscle relaxation (e.g. to increase vascular blood flow); (c) nerve cell modulation; (d) reduction of cell proliferation; (e) reduction of cell migration; (f) suppression of cytoskeletal rearrangement upon pathogen invasion or infection; (g) acceleration of tissue regeneration; and (h) enhancement of post-traumatic functional recovery.

In such embodiments, the nerve cell modulation may comprise: (a) neuronal regeneration; (b) new axonal growth induction; (c) axonal rewiring across lesions within the CNS; (d) neurite outgrowth; (e) neurite differentiation; (f) axon pathfinding; (g) dendritic spine formation; (h) dendritic spine maintenance; (i) modulation of neurite growth cone collapse; and (j) modulation of neurite outgrowth inhibition.

Other applicable treatments include transplantation therapy (e.g. comprising graft protection).

Yet other applicable methods comprise a method of diagnosis and treatment of a disease state or condition, which method comprises: (i) screening a patient to determine whether a disease or condition from which the patient is or may be suffering is one which would be susceptible to treatment with a compound having activity against ROCK kinase; and (ii) where it is indicated that the disease or condition from which the patient is thus susceptible, thereafter administering to the patient a compound according to the invention.

### Applicable Interventions, Treatments and Prophylactic Methods Related to P70S6K Modulation

The invention contemplates protein kinase p70S6K-mediated intervention, treatment or prophylaxis of any kind. Thus, the invention finds application in relation to treatment or prophylaxis comprising: (a) the modulation (e.g. inhibition) of protein kinase p70S6K; (b) intervention at the level of the activity of protein kinase p70S6K; (b) inhibition of progression from G1 to S phase in the cell cycle in vivo; (c) inhibition of cell cycle proliferation at the G1 to S phase of the cell cycle; (d) use of a compound of formula (I) as a rapamycin surrogate; (e) use of a compound of formula (I) as a wortmannin surrogate; (f) the re-establishment of appropriate apoptotic programmes; (g) the inhibition of growth factor receptor signal transduction, overexpression and activation in tumour tissue; (h) modulation of neuronal cell differentiation; (i) modulation of cell motility; (j) modulation of cellular response(s); and (k) enhancing insulin sensitivity.

The treatment or prophylaxis may also comprise a method of diagnosis and treatment of a disease state or condition, which method comprises: (i) screening a patient to determine whether a disease or condition from which the patient is or may be suffering is one which would be susceptible to treatment with a compound having activity against protein kinase p70S6K; and (ii) where it is indicated that the disease or condition from which the patient is thus susceptible, thereafter administering to the patient a compound of formula (I) as herein defined.

### Target Subjects or Patient Populations for ROCK Kinase Modulation

The subject or patient population may be selected from: (a) those in which ROCK kinase is dysfunctional (for example, hyperactive); and (b) those which have been subject to diagnostic tests for ROCK dysfunction (e.g. for ROCK hyperactivity); (c) those in which the Rho signalling pathway is dysfunctional; and (d) those which have been subject to diagnostic tests for Rho signalling pathway dysfunction.

### Target Subjects or Patient Populations for P70S6K Modulation

The subject or patient population may be selected from: (a) those in which protein kinase p70S6K is dysfunctional (for example, hyperactive); (b) those which have been subject to diagnostic tests for p70S6K is dysfunction (e.g. for p70S6K hyperactivity); (c) those in which chromosome 17q23 is amplified; and (d) those which have been subject to diagnostic tests for amplification of chromosome 17q23; (e) those in which BRCA1 mutation(s) are present; (f) those which have been subject to diagnostic tests for BRCA1 mutation(s); (g) those in which BRCA2 mutation(s) are present; (h) those which have been subject to diagnostic tests for BRCA2 mutation(s); (i) those in which LKB1 mutation(s) are present; (j) those which have been subject to diagnostic tests for LKB1 mutation(s); and (k) those which have been screened as defined herein.

### Methods of Treatment and Posology

The compounds of the formula (I) and sub-groups as defined herein will be useful in the prophylaxis or treatment of a range of disease states or conditions mediated by ROCK kinase or protein kinase p70S6K. Examples of such disease states and conditions are set out herein.

Compounds of the formula (I) are generally administered to a subject in need of such administration, for example a human or animal patient, preferably a human.

The compounds will typically be administered in amounts that are therapeutically or prophylactically useful and which generally are non-toxic. However, in certain situations (for example in the case of life threatening diseases), the benefits of administering a compound of the formula (I) may outweigh the disadvantages of any toxic effects or side effects, in which case it may be considered desirable to administer compounds in amounts that are associated with a degree of toxicity.

The compounds may be administered over a prolonged term to maintain beneficial therapeutic effects or may be administered for a short period only. Alternatively they may be administered in a pulsatile or continuous manner.

A typical daily dose of the compound of formula (I) can be in the range from 100 picograms to 100 milligrams per kilogram of body weight, more typically 5 nanograms to 25 milligrams per kilogram of bodyweight, and more usually 10 nanograms to 15 milligrams per kilogram (e.g. 10 nanograms to 10 milligrams, and more typically 1 microgram per kilogram to 20 milligrams per kilogram, for example 1 microgram to 10 milligrams per kilogram) per kilogram of bodyweight although higher or lower doses may be administered where required. The compound of the formula (I) can be administered on a daily basis or on a repeat basis every 2, or 3, or 4, or 5, or 6, or 7, or 10 or 14, or 21, or 28 days for example.

The compounds for use according to the invention may be administered orally in a range of doses, for example 1 to 1500 mg, 2 to 800 mg, or 5 to 500 mg, e.g. 2 to 200 mg or 10 to 1000 mg, particular examples of doses including 10, 20, 50 and 80 mg. The compound may be administered once or more than once each day. The compound can be administered continuously (i.e. taken every day without a break for the duration of the treatment regimen). Alternatively, the compound can be administered intermittently, i.e. taken continuously for a given period such as a week, then discontinued for a period such as a week and then taken continuously for another period such as a week and so on throughout the duration of the treatment regimen. Examples of treatment regimens involving intermittent administration include regimens wherein administration is in cycles of one week on, one week off; or two weeks on, one week off; or three weeks on, one week off; or two weeks on, two weeks off; or four weeks on two weeks off; or one week on three weeks off—for one or more cycles, e.g. 2, 3, 4, 5, 6, 7, 8, 9 or 10 or more cycles.

In one particular dosing schedule, a patient will be given an infusion of a compound of the formula (I) for periods of one hour daily for up to ten days in particular up to five days for one week, and the treatment repeated at a desired interval such as two to four weeks, in particular every three weeks.

More particularly, a patient may be given an infusion of a compound of the formula (I) for periods of one hour daily for 5 days and the treatment repeated every three weeks.

In another particular dosing schedule, a patient is given an infusion over 30 minutes to 1 hour followed by maintenance infusions of variable duration, for example 1 to 5 hours, e.g. 3 hours.

In a further particular dosing schedule, a patient is given a continuous infusion for a period of 12 hours to 5 days, an in particular a continuous infusion of 24 hours to 72 hours.

Ultimately, however, the quantity of compound administered and the type of composition used will be commensurate with the nature of the disease or physiological condition being treated and will be at the discretion of the physician.

The compounds as defined herein can be administered as the sole therapeutic agent or they can be administered in combination therapy with one of more other compounds for treatment of a particular disease state, for example a neoplastic disease such as a cancer as hereinbefore defined. Examples of other therapeutic agents or treatments that may be administered together (whether concurrently or at different time intervals) with the compounds of the formula (I) include but are not limited to:
Topoisomerase I inhibitors
Antimetabolites

Tubulin targeting agents
DNA binder and topoisomerase II inhibitors
Alkylating Agents
Monoclonal Antibodies.
Anti-Hormones
Signal Transduction Inhibitors
Proteasome Inhibitors
DNA methyl transferases
Cytokines and retinoids
Chromatin targeted therapies
Radiotherapy, and,
Other therapeutic or prophylactic agents; for example agents that reduce or alleviate some of the side effects associated with chemotherapy. Particular examples of such agents include anti-emetic agents and agents that prevent or decrease the duration of chemotherapy-associated neutropenia and prevent complications that arise from reduced levels of red blood cells or white blood cells, for example erythropoietin (EPO), granulocyte macrophage-colony stimulating factor (GM-CSF), and granulocyte-colony stimulating factor (G-CSF). Also included are agents that inhibit bone resorption such as bisphosphonate agents e.g. zoledronate, pamidronate and ibandronate, agents that suppress inflammatory responses (such as dexamethazone, prednisone, and prednisolone) and agents used to reduce blood levels of growth hormone and IGF-I in acromegaly patients such as synthetic forms of the brain hormone somatostatin, which includes octreotide acetate which is a long-acting octapeptide with pharmacologic properties mimicking those of the natural hormone somatostatin. Further included are agents such as leucovorin, which is used as an antidote to drugs that decrease levels of folic acid, or folinic acid it self and agents such as megestrol acetate which can be used for the treatment of side-effects including oedema and thromoembolic episodes.

Each of the compounds present in the combinations may be given in individually varying dose schedules and via different routes.

Where the compound of the formula (I) is administered in combination therapy with one, two, three, four or more other therapeutic agents (preferably one or two, more preferably one), the compounds can be administered simultaneously or sequentially. When administered sequentially, they can be administered at closely spaced intervals (for example over a period of 5-10 minutes) or at longer intervals (for example 1, 2, 3, 4 or more hours apart, or even longer periods apart where required), the precise dosage regimen being commensurate with the properties of the therapeutic agent(s).

The compounds for use according to the invention may also be administered in conjunction with non-chemotherapeutic treatments such as radiotherapy, photodynamic therapy, gene therapy; surgery and controlled diets.

For use in combination therapy with another chemotherapeutic agent, the compound of the formula (I) and one, two, three, four or more other therapeutic agents can be, for example, formulated together in a dosage form containing two, three, four or more therapeutic agents. In an alternative, the individual therapeutic agents may be formulated separately and presented together in the form of a kit, optionally with instructions for their use.

A person skilled in the art would know through his or her common general knowledge the dosing regimes and combination therapies to use.

### Methods of Diagnosis

Prior to administration of a compound of the formula (I) as defined herein, a patient may be screened to determine whether a disease or condition from which the patient is or may be suffering is one which would be susceptible to treatment. For example, the patient may be screened for dysfunction in ROCK activity (e.g. elevated or up-regulated ROCK expression, mutations in ROCK genes or ROCK gene regulatory elements) or Rho signalling dysfunction (as described herein).

The term up-regulation includes elevated expression or over-expression, including gene amplification (i.e. multiple gene copies) and increased expression by a transcriptional effect, and hyperactivity and activation, including activation by mutations. The term diagnosis includes screening. By marker we include genetic markers including, for example, the measurement of DNA composition to identify mutations. The term marker also includes markers which are characteristic of up regulation including enzyme activity, enzyme levels, enzyme state (e.g. phosphorylated or not) and mRNA levels.

The above diagnostic tests and screens are typically conducted on a biological sample selected from tumour biopsy samples, blood samples (isolation and enrichment of shed tumour cells), stool biopsies, sputum, chromosome analysis, pleural fluid, peritoneal fluid, or urine.

Identification of an individual carrying a mutation may mean that the patient would be particularly suitable for treatment according to the invention. Tumours may preferentially be screened for presence of a particular mutation/allele prior to treatment. The screening process will typically involve direct sequencing, oligonucleotide microarray analysis, or a mutant specific antibody.

Methods of identification and analysis of mutations and up-regulation of proteins are known to a person skilled in the art. Screening methods could include, but are not limited to, standard methods such as reverse-transcriptase polymerase chain reaction (RT-PCR) or in-situ hybridisation.

In screening by RT-PCR, the level of mRNA in the tumour is assessed by creating a cDNA copy of the mRNA followed by amplification of the cDNA by PCR. Methods of PCR amplification, the selection of primers, and conditions for amplification, are known to a person skilled in the art. Nucleic acid manipulations and PCR are carried out by standard methods, as described for example in Ausubel, F. M. et al., eds. Current Protocols in Molecular Biology, 2004, John Wiley & Sons Inc., or Innis, M. A. et-al., eds. PCR Protocols: a guide to methods and applications, 1990, Academic Press, San Diego. Reactions and manipulations involving nucleic acid techniques are also described in Sambrook et al., 2001, 3^{rd} Ed, Molecular Cloning: A Laboratory Manual, Cold Spring Harbor Laboratory Press. Alternatively a commercially available kit for RT-PCR (for example Roche Molecular Biochemicals) may be used, or methodology as set forth in U.S. Pat. Nos. 4,666,828; 4,683,202; 4,801,531; 5,192,659, 5,272,057, 5,882,864, and 6,218,529 and incorporated herein by reference.

An example of an in-situ hybridisation technique for assessing mRNA expression would be fluorescence in-situ hybridisation (FISH) (see Angerer, 1987 Meth. Enzymol., 152: 649).

Generally, in situ hybridization comprises the following major steps: (1) fixation of tissue to be analyzed; (2) prehybridization treatment of the sample to increase accessibility of target nucleic acid, and to reduce nonspecific binding; (3) hybridization of the mixture of nucleic acids to the nucleic acid in the biological structure or tissue; (4) post-hybridization washes to remove nucleic acid fragments not bound in the hybridization, and (5) detection of the hybridized nucleic acid fragments. The probes used in such applications are typically labeled, for example, with radioisotopes or fluorescent reporters. Preferred probes are sufficiently long, for example, from about 50,100, or 200 nucleotides to about 1000 or more nucleotides, to enable specific hybridization with the target nucleic acid(s) under stringent conditions. Standard methods for carrying out FISH are described in Ausubel, F. M. et al., eds. Current Protocols in Molecular Biology, 2004, John Wiley & Sons Inc and Fluorescence In Situ Hybridization: Technical Overview by John M. S. Bartlett in Molecular Diagnosis of Cancer, Methods and Protocols, 2nd ed.; ISBN: 1-59259-760-2; March 2004, pps. 077-088; Series: Methods in Molecular Medicine.

Alternatively, the protein products expressed from the mRNAs may be assayed by immunohistochemistry of tumour samples, solid phase immunoassay with microtitre plates,

Western blotting, 2-dimensional SDS-polyacrylamide gel electrophoresis, ELISA, flow cytometry and other methods known in the art for detection of specific proteins. Detection methods would include the use of site specific antibodies. The skilled person will recognize that all such well-known techniques could be applicable in the present case.

### Particular Considerations Arising in Respect of LKB1

DNA sequencing is a viable method of genetic testing for LKB1 mutation in the diagnostic laboratory (see for example J Med Genet (1999) 36: 365-368). This paper describes the screening of a set of 12 Peutz-Jeghers patients for germline mutations in LKB1 and report the results of this screening. Such protocols find application in the present invention.

Further details of appropriate protocols may be found for example in Shaw et al. (2004) Cancer Cell 6: 91-99 (which describes how the LKB1 tumor suppressor negatively regulates mTOR signaling) and in Jimenez et al. (2003) Cancer Res. 63: 1382-1388.

### Amplification and Detection of ROCK Kinase

Detection of ROCK may be carried out at either the mRNA or protein level.

Specific examples of methods where levels of Rho and ROCK have been determined in clinical samples include:
American Journal of Pathology. 2002; 160:579-584. This paper describes immunohistochemistry performed on formalin-fixed tissues to characterize RhoC expression in human breast tissues.
Clinical Cancer Research Vol. 9, 2632-2641, July 2003. This paper describes the use of Western blotting to quantitate Rho and ROCK protein expression in paired tumour and nontumour surgical samples from 107 consecutive Japanese patients with bladder cancer.
Pancreas. 24(3):251-257, April 2002. This paper describes the expression of ROCK-1 in human pancreatic tissues by immunoblotting and immunohistochemistry.
World J Gastroenterol 2003 September; 9(9):1950-1953. This paper describes the examination of mRNA expression levels of RhoC gene by reverse transcription-polymerase chain reaction (RT-PCR) in hepatocellular carcinoma (HCC).

The relevant methodological disclosure relating to the quantitation of the levels of Rho and/or ROCK activity or expression contained in the above-mentioned publications are hereby incorporated herein by reference.

### Amplification and Detection of Protein Kinase P70S6K

Detection of p70S6K may be carried out at either the mRNA or protein level.

Exemplary methods are described for example in J Naltl Cancer Inst (2000): 92, pp. 1252-9 (which describes detecting the activation of Ribosomal Protein S6 Kinase by complementary DNA and tissue microarray analysis uses comparative genomic hybridization (CGH) and cDNA and tissue microarray analyses to identify amplified and overexpressed genes).

The detection of overexpressed p70S6K is described in Int J Oncol (2004): 24 (4), pp. 893-900. This paper describes the pharmacolgenomic profiling of the PI3K/PTEN-Akt-mTOR pathway in common human tumours using immunohistoochemistry to compare high p70S6K, AKT expression to tumour sensitivity.

### EXPERIMENTAL

The invention will now be illustrated, but not limited, by reference to the specific embodiments described in the following procedures and examples.

The starting materials for each of the procedures described below are commercially available, or are readily prepared from commercially available materials, unless otherwise specified.

Proton magnetic resonance (¹H NMR) spectra were recorded on a Bruker AV400 instrument operating at 400.13 MHz, in Me-d₃-OD at 27C, unless otherwise stated and are reported as follows: chemical shift δ/ppm (number of protons, multiplicity where s=singlet, d=doublet, t=triplet, q=quartet, m=multiplet, br=broad). The residual protic solvent MeOH (δ_{H}=3.31 ppm) was used as the internal reference.

In the examples, the compounds prepared were characterised by liquid chromatography and mass spectroscopy using the systems and operating conditions set out below. Where chlorine is present, the mass quoted for the compound is for ³⁵Cl. The operating conditions used are described below.

### FractionLynx System

| | |
|---|---|
| System: | Waters FractionLynx (dual analytical/prep) |
| HPLC Pump: | Waters 2525 |
| Injector-Autosampler: | Waters 2767 |
| Mass Spec Detector: | Waters-Micromass ZQ |
| PDA Detector: | Waters 2996 PDA |

Acidic Analytical conditions:

| | |
|---|---|
| Eluent A: | H₂O (0.1% Formic Acid) |
| Eluent B: | CH₃CN (0.1% Formic Acid) |
| Gradient: | 5-95% eluent B over 5 minutes |
| Flow: | 2.0 ml/min |
| Column: | Phenomenex Synergi 4μ Max-RP 80 A, 50 × |
| | 4.6 mm |

MS conditions:

| | |
|---|---|
| Capillary voltage: | 3.5 kV |
| Cone voltage: | 25 V |
| Source Temperature: | 120° C. |
| Scan Range: | 125-800 amu |
| Ionisation Mode: | ElectroSpray Positive or ElectroSpray |
| | Positive & Negative |

### Platform System

| | |
|---|---|
| HPLC System: | Waters 2795 |
| Mass Spec Detector: | Micromass Platform LC |
| PDA Detector: | Waters 2996 PDA |

Polar Analytical conditions:

| | |
|---|---|
| Eluent A: | H₂O (0.1% Formic Acid) |
| Eluent B: | CH₃CN (0.1% Formic Acid) |
| Gradient: | 00-50% eluent B over 3 minutes |
| Flow: | 1.5 ml/min |
| Column: | Phenomenex Synergi 4μ Hydro 80 A, 50 × |
| | 4.6 mm |

MS conditions:

| | |
|---|---|
| Capillary voltage: | 3.5 kV |
| Cone voltage: | 30 V |
| Source Temperature: | 120° C. |
| Scan Range: | 165-700 amu |
| Ionisation Mode: | ElectroSpray Negative, Positive or |
| | Positive & Negative |

Acidic Analytical conditions:

| | |
|---|---|
| Eluent A: | H₂O (0.1% Formic Acid) |
| Eluent B: | CH₃CN (0.1% Formic Acid) |
| Gradient: | 5-95% eluent B over 3.5 minutes |
| Flow: | 0.8 ml/min |
| Column: | Phenomenex Synergi 4μ Max-RP 80 A, 50 × |
| | 2.0 mm |

### LCT System 1

| | |
|---|---|
| HPLC System: | Waters Alliance 2795 Separations Module |
| Mass Spec Detector: | Waters/Micromass LCT |
| UV Detector: | Waters 2487 Dual λ Absorbance Detector |

Polar Analytical conditions:

| Eluent A: | Methanol | | |
|---|---|---|---|
| Eluent B: | 0.1% Formic Acid in Water | | |
| Gradient: | Time (mins) | A | B |
| | 0 | 10 | 90 |
| | 0.5 | 10 | 90 |
| | 6.5 | 90 | 10 |
| | 10 | 90 | 10 |
| | 10.5 | 10 | 90 |
| | 15 | 10 | 90 |

| | |
|---|---|
| Flow: | 1.0 ml/min |

| | |
|---|---|
| Column: | Supelco DISCOVERY C₁₈ 5 cm × 4.6 mm i.d., |
| | 5 μm |

MS conditions:

| | |
|---|---|
| Capillary voltage: | 3500 v (+ve ESI), 3000 v (−ve ESI) |
| Cone voltage: | 40 v (+ve ESI), 50 v (−ve ESI) |
| Source Temperature: | 100° C. |
| Scan Range: | 50-1000 amu |
| Ionisation Mode: | +ve/−ve electrospray ESI (Lockspray ™) |

### LCT System 2

| | |
|---|---|
| HPLC System: | Waters Alliance 2795 Separations Module |
| Mass Spec Detector: | Waters/Micromass LCT |
| UV Detector: | Waters 2487 Dual λ Absorbance Detector |

Analytical conditions:

| Eluent A: | Methanol | | |
|---|---|---|---|
| Eluent B: | 0.1% Formic Acid in Water | | |
| Gradient: | Time (mins) | A | B |
| | 0 | 10 | 90 |
| | 0.6 | 10 | 90 |
| | 1.0 | 20 | 80 |
| | 7.5 | 90 | 10 |
| | 9 | 90 | 10 |
| | 9.5 | 10 | 90 |
| | 10 | 10 | 90 |

| | |
|---|---|
| Flow: | 1 ml/min |

| | |
|---|---|
| Column: | Supelco DISCOVERY C₁₈ 5 cm × 4.6 mm i.d., |
| | 5 μm |

MS conditions:

| | |
|---|---|
| Capillary voltage: | 3500 v (+ve ESI), 3000 v (−ve ESI) |
| Cone voltage: | 40 v (+ve ESI), 50 v (−ve ESI) |
| Source Temperature: | 100° C. |
| Scan Range: | 50-1000 amu |
| Ionisation Mode: | +ve/−ve electrospray ESI (Lockspray ™) |

### Agilent System

| | |
|---|---|
| HPLC System: | Agilent 1100 series |
| Mass Spec Detector: | Agilent LC/MSD VL |
| Multi Wavelength | Agilent 1100 series MWD |
| Detector: | |
| Software: | HP Chemstation |

Chiral Analytical conditions:

| | |
|---|---|
| Eluent: | MeOH + 0.1% NH4/AcOH at room Temperature |
| Flow: | 1.0 ml/min |
| Total time: | 60.0 min |
| Inj. Volume: | 20 uL |
| Sample Conc: | 2 mg/ml |
| Column: | Astec, Chirobiotic V; 250 × 4.6 mm |

Chiral Preparative conditions 1:

| | |
|---|---|
| Eluent: | MeOH + 0.1% NH4/TFA at room Temperature |
| Flow: | 6.0 ml/min |
| Total time: | 50 min |
| Inj. Volume: | 50 uL |
| Sample Conc: | 20 mg/ml |
| Column: | Astec, Chirobiotic V; 250 × 10 mm |

In the examples below, the following key is used to identify the LCMS conditions used:

| | |
|---|---|
| PS-P | Platform System - polar analytical conditions |
| PS-A | Platform System - acid analytical conditions |
| FL-A | FractionLynx System - acidic analytical conditions |
| LCT1 | LCT System 1 - polar analytical conditions |
| LCT2 | LCT System 2 - polar analytical conditions |
| AS-CA | Agilent system - chiral analytical conditions |

### Example 1

### N-Methyl-N′-(9H-purin-6-yl)-propane-1,3-diamine

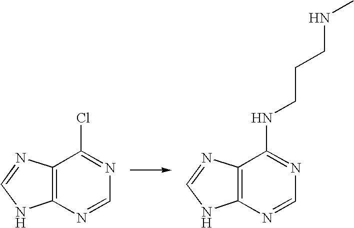

A solution of 6-chloropurine (0.3 g, 1.94 mmol) and N-methyl-1,3-propanediamine (0.61 ml, 5.82 mmol) in ethanol (5 ml) was heated at 120° C. (100 W) for 15 minutes in a sealed microwave tube with stirring in a CEM Discover microwave. Solvent was removed under reduce pressure and the residue was purified over flash silica chromatography eluting with methanol/dichloromethane (2:8) to yield the title compound as a white solid (0.197 g, 49% yield). LC/MS: (FL-A) Rₜ 0.36 [M+H]⁺ 207.22. ¹H NMR (DMSO) 1.92-2.03 (2H, m), 2.52 (2H, t), 2.81 (2H, t), 8.14 (1H, s), 8.20 (1H, s).

### Example 2

### 6-(3-Methylamino-propylamino)-7,9-dihydro-purin-8-one

### 2A. N-(8-Bromo-9H-purin-6-yl)-N′-methyl-propane-1,3-diamine

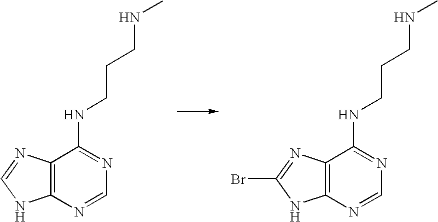

N-Bromosuccinimide (0.86 g, 4.84 mmol) was added to a solution of N-Methyl-N′-(9H-purin-6-yl)-propane-1,3-diamine (0.2 g, 0.97 mmol) in acetonitrile and the reaction mixture was stirred at room temperature for 64 hours. The solvent was removed under reduced pressure and the residue was purified over flash silica chromatography eluting with dichloromethane/methanol/acetic acid/water (90:18:3:2) to afford the title compound (0.044 g, 16% yield). LC/MS: (PS-P) Rₜ 1.72 [M+H]⁺ 284.93, 286.93.

### 2B. Methyl-[3-(8-oxo-8,9-dihydro-7H-purin-6-ylamino)-propyl]-carbamic acid tert-butyl ester

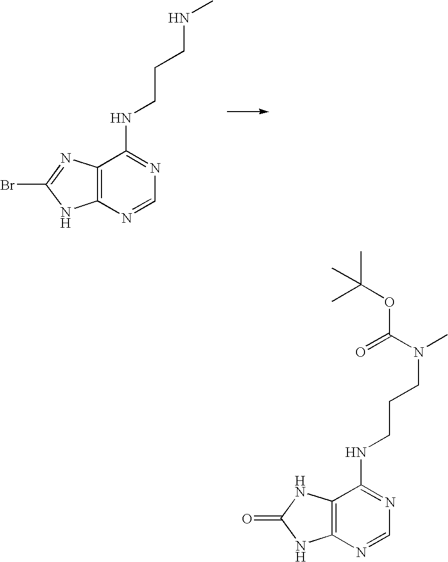

A solution of N-(8-bromo-9H-purin-6-yl)-N′-methyl-propane-1,3-diamine (0.04 g, 0.14 mmol) in concentrated hydrochloric acid (1 ml) was heated at 100° C. for 16 hours. The reaction mixture was transferred to iced water, neutralised with 2N sodium hydroxide, di-tert-butyl carbonate (0.03 g, 0.17 mmol) in tetrahydrofuran (1.5 ml) and sodium hydroxide (0.01 g, 0.14 mmol) were added. The reaction mixture was stirred for 1 hour, extracted with ethyl acetate. The organic layer was washed with brine, dried (MgSO₄) and solvent removed under reduced pressure. Purified over flash silica chromatography eluting with methanol/dichloromethane (5:95) to afford the title compound as a white solid (0.042 g, 93% yield). LC/MS: (PS-P) Rₜ 2.56 [M+H]⁺ 323.08.

### 2C. 6-(3-Methylamino-propylamino)-7,9-dihydro-purin-8-one

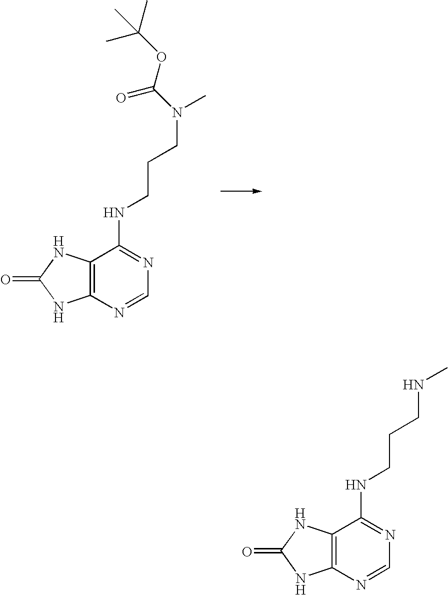

Methyl-[3-(8-oxo-8,9-dihydro-7H-purin-6-ylamino)-propyl]-carbamic acid tert-butyl ester (0.042 g, 0.13 mmol) was treated with 4M HCl in dioxane. The reaction mixture was stirred for 2 hours, solvent removed under reduced pressure to yield the title compound as a white solid (0.01 g, 35% yield). LC/MS: (PS-P) Rₜ 1.55 [M+H]⁺ 223.05. ¹H NMR (Me-d₃-OD) 2.04-2.13 (2H, m), 3.03 (2H, t), 3.45 (3H, s), 3.87 (2H, t), 8.32 (1H, s).

The following compounds were prepared in a similar manner:

### Example 3

### 1-(4-Fluorophenyl)-N³-(9H-purin-6-yl)propane-1,3-diamine

### 3A. [1-(4-Fluorophenyl)-3-(9H-purin-6-ylamino)propyl]carbamic acid tert-butyl ester

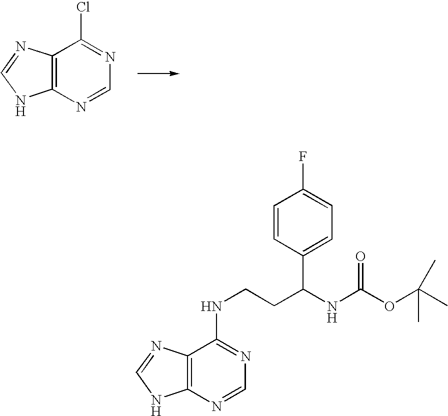

6-Chloropurine was reacted with [3-amino-1-(4-fluoro-phenyl)-propyl]-carbamic acid tert-butyl ester (Pharmacore, Inc, NC, USA) under the conditions described in Example 1A using a 2-fold excess of the amine and 5 equivalents of triethylamine to give the title compound: LC/MS: (LCT1) Rₜ 5.87 [M+H]⁺ 387.

### 3B. 1-(4-Fluorophenyl)-N³-(9H-purin-6-yl)propane-1,3-diamine

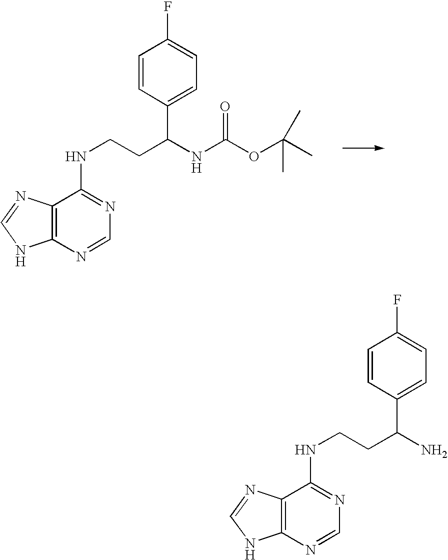

Removal of the Boc protecting group was accomplished using the method described in Example 2C to give the title compound: LC/MS: (LCT1) Rₜ 2.52 [M-NH₂]⁺ 270.

### Example 4

### 6-[3-Amino-3-(4-fluorophenyl)propylamino]-7,9-dihydropurin-8-one

### 4A. [3-(8-Bromo-9H-purin-6-ylamino)-1-(4-fluorophenyl)propyl]carbamic acid tert-butyl ester

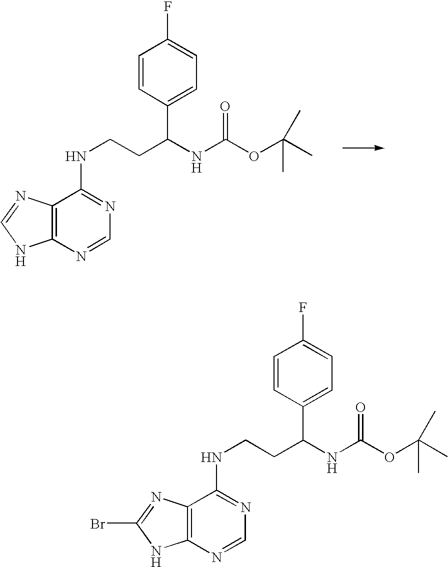

The product of Example 3A was brominated using N-bromosuccinimide according to the method of Example 2A to give the title compound: LC/MS: (LCT1) Rₜ 6.64 [M+H]⁺ 465.

### 4B. 6-[3-Amino-3-(4-fluorophenyl)propylamino]-7,9-dihydropurin-8-one

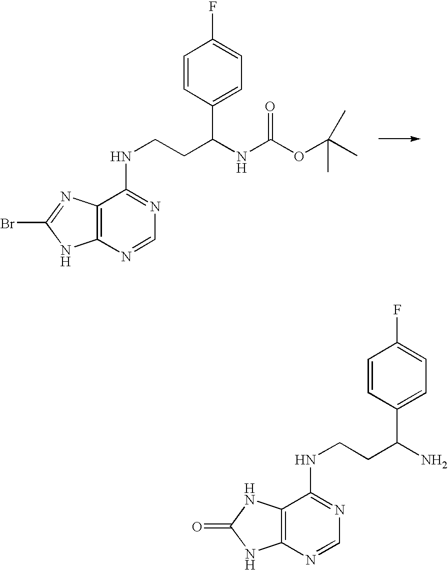

The bromo-compound of Example 4A was subjected to hydrolysis in hydrochloric acid using the method of Example 2B to give the title compound: LC/MS: (LCT1) Rₜ 3.05 [M-NH₂]+286.

### Example 5

### 1-(4-Chlorophenyl)-N³-(9H-purin-6-yl)propane-1,3-diamine

### 5A. [1-(4-Chlorophenyl)-3-(9H-purin-6-ylamino)propyl]carbamic acid tert-butyl ester

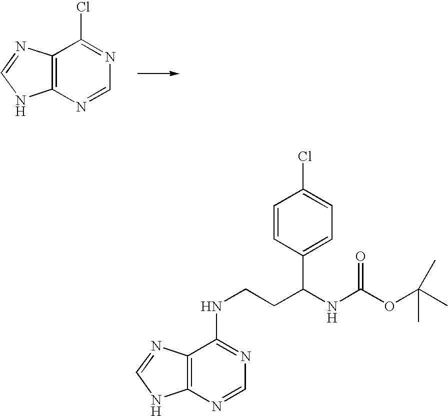

6-Chloropurine was reacted with [3-amino-1-(4-chloro-phenyl)-propyl]-carbamic acid tert-butyl ester (Pharmacore Inc, NC, USA) according to the method described in Example 1 to give the title compound: LC/MS: (LCT1) Rₜ 6.49 [M+H]⁺ 403.

### 5B. 1-(4-Chlorophenyl)-N-3-(9H-purin-6-yl)propane-1,3-diamine

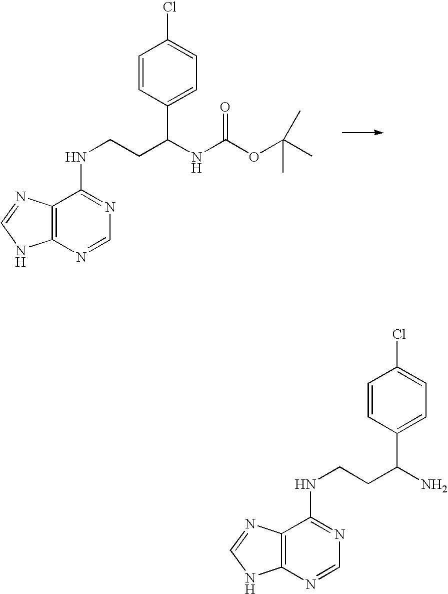

The product of Example 5A was deprotected by the method of Example 2C to give the title compound: LC/MS: (LCT1) Rₜ 3.02 [M-NH₂]⁺ 286.

### Example 6

### Methyl-(4-(9H-purin-6-yl)benzyl)amine

### 6A. 4-(9-(Tetrahydropyran-2-yl)-9H-purin-6-yl)benzaldehyde

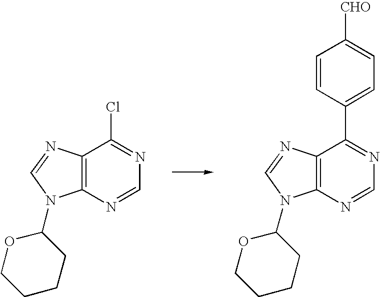

A mixture of 9-(tetrahydropyran-2-yl)-6-chloropurine (J. Am. Chem. Soc. 1961, 2574) (0.13 g, 0.55 mmol), 4-formylboronic acid (0.11 g, 0.75 mmol), 2M K₂CO₃ aq. (0.70 ml, 1.4 mmol) and Pd(PPh₃)₄ (0.03 g, 5 mol %) in 1,2-dimethoxy ethane (DME) (5 ml) was degassed and flushed with argon. The yellow solution was stirred at 85° C. under argon for 24 h, then cooled and filtered through Celite®, washing with EtOAc. The filtrate was concentrated and purified by flash column chromatography on silica gel, eluting with 50% EtOAc-hexanes, to give an off-white solid (0.354g, 64%). LC/MS: (LCT1) Rₜ 6.15 [M+H-THP]⁺ 225

### 6B. Methyl-(4-(9-(tetrahydropyran-2-yl)-9H-purin-6-yl)benzyl)amine

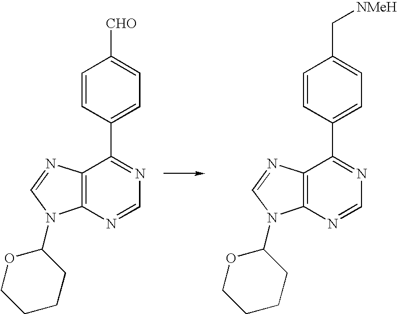

A solution of the aldehyde of Example 6A (0.25 g, 0.812 mmol) and methylamine (33% in EtOH, 25 ml) was stirred at room temperature for 2 hours, followed by evaporation of the solvent and excess amine. The white solid was redissolved in MeOH (25 ml) and NaBH₄ (0.05 g, 1.32 mmol) was added. After 30 minutes the solution was diluted with water (200 ml) and extracted with CH₂Cl₂ (100 ml). The extract was dried (Na₂SO₄), filtered and concentrated to give the amine as a colourless gum (0.231 g, 88%). LC/MS (LCT1): Rₜ 3.94 [M+H]⁺ 325.

### 6C. Methyl-(4-(9H-purin-6-yl)benzyl)amine

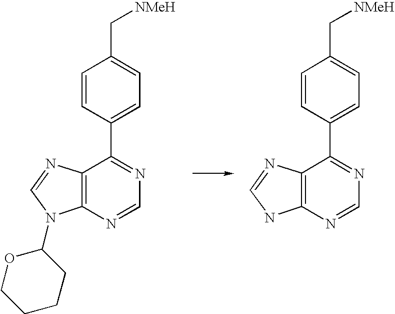

A solution of the amine of Example 6B in EtOH (15 ml) and 1 M HCl (10 ml) was stirred at room temperature for 16 hours and was then evaporated to dryness. Solid phase extraction on SCX-II acidic resin, eluting with MeOH then 1M NH₃ in MeOH, gave the deprotected amine as a cream-coloured solid (0.142 g, 83%). LC/MS (LCT1): Rₜ 2.43 [M+H]⁺ 240.

### Example 7

### Methyl-(3-(9H-purin-6-yl)benzyl)amine

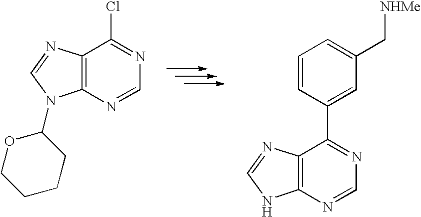

Starting from 6-chloro-9-(tetrahydro-pyran-2-yl)-9H-purine and 3-formylboronic acid and following the procedures set out in Example 6 gave the title compound: LC/MS (LCT1): Rₜ 2.77 [M+H]⁺ 240

### Example 8

### (4-(9H-purin-6-yl)phenyl)acetonitrile

### 8A. (4-(9-(Tetrahydropyran-2-yl)-9H-purin-6-yl)phenyl)acetonitrile

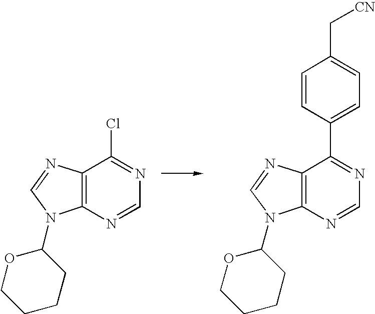

A solution of the N-protected chloropurine (0.27 g, 1.12 mmol), 4-cyanomethylphenylboronic acid (0.22 g, 1.37 mmol), 2M K₂CO₃ aq. (1.4 ml, 2.8 mmol) and Pd(PPh₃)₄ (0.03 g, 2.5 mol %) in DME (4 ml) was irradiated in a microwave reactor at 150° C. for 25 minutes. The organic layer was absorbed onto silica gel and purified by flash column chromatography, eluting 50% EtOAc-hexanes, to give a yellow solid (0.25 g, 70%). LC/MS (LCT1): Rₜ 5.84 [M+H-THP]+236.

### 8B. (4-(9H-purin-6-yl)phenyl)acetonitrile

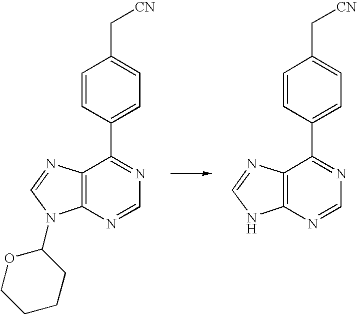

A mixture of the protected purine product of Example 8A (0.026 g, 0.081 mmol) and 1 M HCl (1 ml) in EtOH (1.5 ml) was stirred at 80° C. for 6 hours and then evaporated to dryness. Filtration through SCX-II acidic resin, eluting with MeOH then 1M NH₃ in MeOH gave the title compound as a cream-coloured solid (0.015 g, 79%). LC/MS (LCT1): Rₜ 4.37 [M+H]⁺ 236.

### Example 9

### 2-(4-(9H-Purin-6-yl)phenyl)ethylamine

### 9A. 2-(4-(9-(Tetrahydropyran-2-yl)-9H-purin-6-yl)phenyl)ethylamine

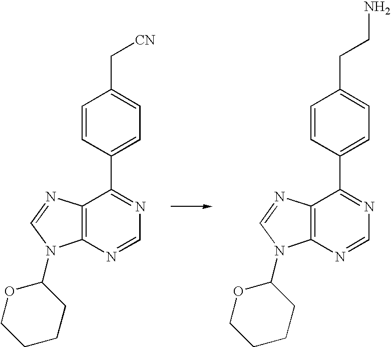

A suspension of Raney nickel in water (0.25 ml) was added to a solution of (4-(9-(tetrahydropyran-2-yl)-9H-purin-6-yl)phenyl)acetonitrile (0.021 g, (0.066 mmol) in 1,4-dioxane (2 ml). The suspension was stirred vigorously at 80° C. and hydrazine hydrate (0.5 ml) was added cautiously. After 30 minutes, the solution was cooled and filtered through SCX-II acidic resin, eluting with MeOH then 1M NH₃ in MeOH, to give a the title compound as a colourless oil (0.021 g, 98%) LC/MS (LCT1): Rₜ 4.22 [M+H-THP]⁺ 240.

### 9B. 2-(4-(9H-Purin-6-yl)phenyl)ethylamine

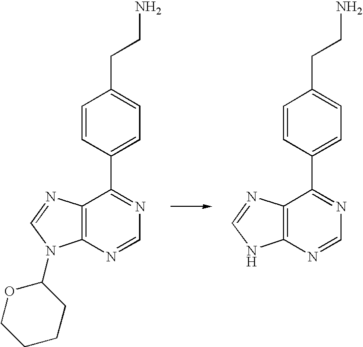

A solution of the protected purine of Example 9A (0.021 g, 0.065 mmol) and 1 M HCl (2 ml) and EtOH (2 ml) was stirred at room temperature for 16 hours and then evaporated to dryness. Filtration through SCX-II acidic resin, eluting with MeOH then 1M NH₃ in MeOH, gave an off-white solid (0.011 g, 71%). LC/MS (LCT1): Rₜ 2.82 [M+H]⁺ 240.

The following compound was prepared by similar methods:

### Example 10

### 2-(3-(9H-purin-6-yl)phenyl)ethylamine

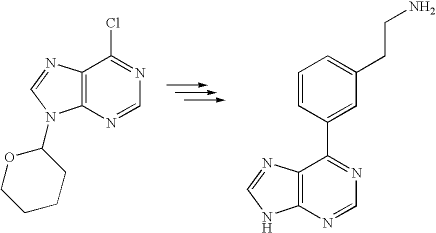

By reacting 6-chloro-9-(tetrahydro-pyran-2-yl)-9H-purine and 4-cyanomethylphenylboronic acid according to the method of Example 8A and then following the reduction and deprotection steps set out in Examples 9A and 9B, the title compound was prepared: LC/MS (LCT1): Rₜ 3.02 [M+H]⁺ 240.

### Example 11

### 1-(9H-Purin-6-yl)piperidine-4-carboxylic acid amide

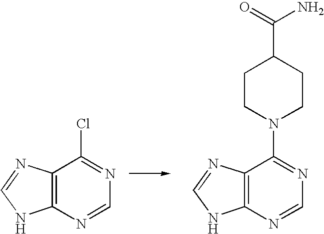

A solution of 6-chloropurine (0.500 g, 3.24 mmol), isonipecotamide (0.829 g, 6.47 mmol) and triethylamine (2.25 ml, 16.2 mmol) in n-butanol (32 ml) was stirred at 100° C. for 40 minutes. The suspension was concentrated and the residue was stirred with methanol (20 ml) for 1 hour. The insoluble white solid was collected and dried in vacuo to give the product (0.775 g, 96%). LC/MS: (LCT1) Rₜ 2.04 [M+H]⁺ 247.

### Example 12

### C-[1-(9H-Purin-6-yl)piperidin-4-yl]methylamine

### 12A. [1-(9H-Purin-6-yl)piperidin-4-ylmethyl]carbamic acid tert-butyl ester

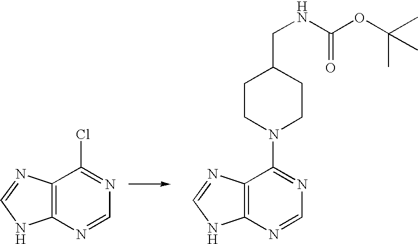

LC/MS: (LCT1) Rₜ 5.42 [M+H]⁺ 332.

### 12B. C-[1-(9H-Purin-6-yl)piperidin-4-yl]methylamine

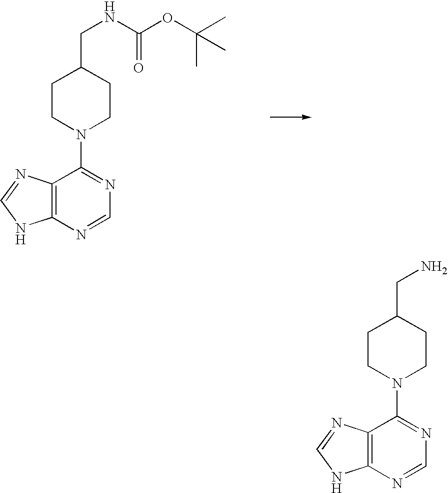

LC/MS (LCT1): Rₜ 1.18 [M+H]⁺ 233.

### Example 13

### 6-[4-(Aminophenylmethyl)piperidin-1-yl]-7,9-dihydropurin-8-one

13A. 5,6-Diamino-4-chloropyrimidine

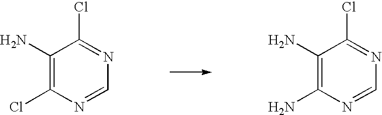

A mixture of 4,6-dichloro-5-aminopyrimidine (Aldrich Chemical Co.) (2.0 g, 12.2 mmol) and concentrated aqueous ammonia (20 ml) was heated to 100° C. in a sealed glass tube with vigorous stirring for 18 hours. The cooled tube was recharged with concentrated aqueous ammonia (8 ml), aggregates were broken up, and the mixture was reheated at 100° C. for a further 28 hours. The mixture was evaporated to dryness and the solids were washed with water (20 ml) and dried to give the product as yellow crystals (1.71 g, 97%). LC/MS (LCT1): Rₜ 1.59 [M+H]⁺ 147, 145.

### 13B. 6-Chloro-7,9-dihydropurin-8-one

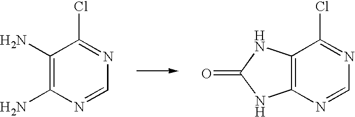

A mixture of the 5,6-diamino-4-chloropyrimidine of Example 13A (1.0 g, 6.92 mmol) and N,N′-carbonyldiimidazole (2.13 g, 13.2 mmol) in 1,4-dioxane (20 ml) was refluxed under argon for 48 hours. The solution was concentrated to a brown oil, which was triturated and washed with dichloromethane to give an off-white solid (1.02 g, 86%) LC/MS (LCT1): Rₜ 2.45 [M+H]⁺ 173, 171.

### 13C. 6-(4-Benzoylpiperidin-1-yl)-7,9-dihydropurin-8-one

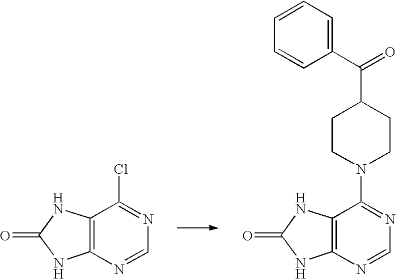

To a mixture of the 6-chloro-7,9-dihydropurin-8-one of Example 13B (0.100 g, 0.586 mmol) and (0.265 g, 1.172 mmol) in n-butanol (5.8 ml) was added triethylamine (0.408 ml, 2.930 mmol). After heating at 100° C. for 24 hours, solvent was removed and the resulting solid was triturated with methanol (10 ml). Filtration gave the title product as a white solid (0.121 g, 64%). LC/MS: (LCT1) Rₜ 5.70 [M+H]⁺ 324.

### 13D. 6-[4-(Aminophenylmethyl)piperidin-1-yl]-7,9-dihydropurin-8-one

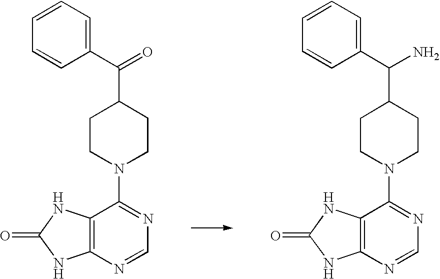

To a solution of the purinone of Example 13C (0.060 g, 0.186 mmol) in methanol (2 ml) was added ammonium acetate (172 mg, 2.227 mmol) and sodium cyanoborohydride (47 mg, 0.742 mmol). After refluxing for 2 days, the solution was cooled, then purified by solid phase extraction on SCX-II acidic resin, eluting with MeOH then 1M NH₃ in MeOH, which gave the title amine as a white solid (0.055 g, 92%). LC/MS (LCT1): Rₜ 3.90 [M+H]⁺ 325

### Example 14

### 6-[4-(Amino(4-chlorophenyl)methyl)piperidin-1-yl]-7,9-dihydropurin-8-one

### 14A. 6-(4-(4-Chlorobenzoyl)piperidin-1-yl)-7,9-dihydropurin-8-one

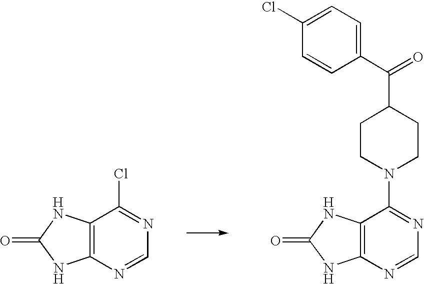

6-Chloro-7,9-dihydropurin-8-one was reacted with 4-(amino(4-chlorophenyl)methyl)piperidine by the method of Example 13C to give the title compound:

LC/MS: (LCT1) Rₜ 6.42 [M+H]⁺ 358.

### 14B. 6-[4-(Amino(4-chlorophenyl)methyl)piperidin-1-yl]-7,9-dihydropurin-8-one

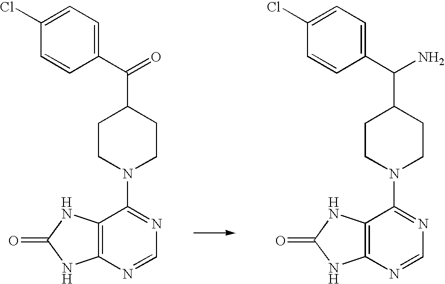

6-(4-(4-Chlorobenzoyl)piperidin-1-yl)-7,9-dihydropurin-8-one was subjected to the reductive amination method of Example 13D to give the title compound: LC/MS (LCT1): Rₜ 4.43 [M+H]⁺ 359.

### Example 15

### 6-(4-Aminomethylpiperidin-1-yl)-7,9-dihydrofurin-8-one

### 15A. [1-(8-Oxo-8,9-dihydro-7H-purin-6-yl)piperidin-4-ylmethyl]carbamic acid tert-butyl ester

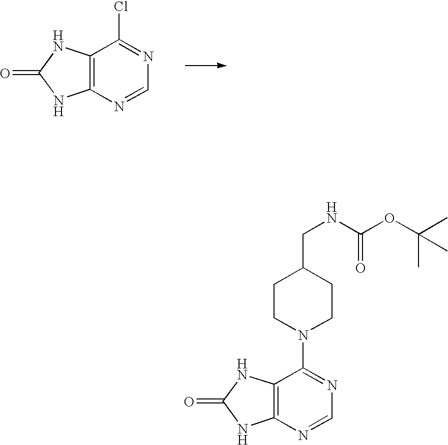

Following the method of Example 13C but using piperidin-4-ylmethylcarbamic acid tert-butyl ester as the amine yielded the title compound: LC/MS: (LCT1) Rₜ 5.70 [M+H]⁺ 349.

### 15B. 6-(4-Aminomethylpiperidin-1-yl)-7,9-dihydropurin-8-one

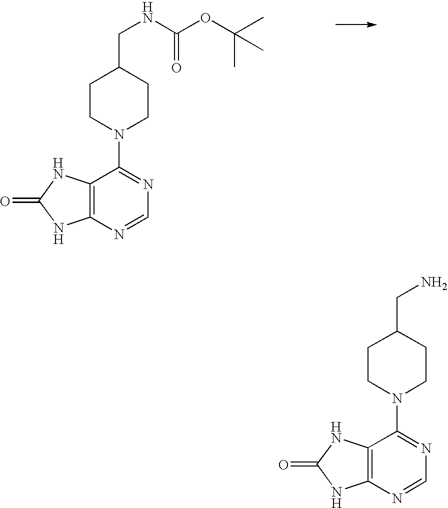

The product of Example 15A was deprotected by the method of Example 2C to give the title compound: LC/MS (LCT1): Rₜ 1.59 [M+H]⁺ 249.

### Example 16

### 3-[3-(9H-Purin-6-yl)-phenoxy]-propylamine

### 16A. [3-(3-Bromo-phenoxy)-propyl]-carbamic acid tert-butyl ester

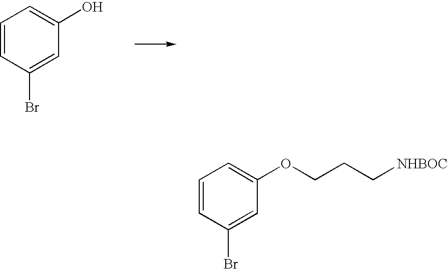

To a solution of 3-bromophenol (4.75 g, 27.2 mmol) in THF (40 ml) were added 3-hydroxypropylcarbamic acid tert-butyl ester (5.75 g, 32.8 mmol) in THF (30 ml) and triphenylphosphine (10.9 g, 41 mmol). The solution was cooled (ice bath) and diisopropylazodicarboxylate (DIAD) (7 ml, 35.5 mmol) was added dropwise. The solution was stirred at room temperature for 48 hours, and then hexane (100 ml) was added. The solution was washed with 1 M NaOH solution (7×50 ml), then dried, concentrated and purified by flash column chromatography (silica gel, 4:1 hexane:ethyl acetate) to yield the product (3.28 g, 35%). ¹H NMR (250 MHz, d6-acetone) 1.42 (9H, s), 1.99 (2H, m), 3.28 (2H, q), 4.09 (2H, t), 6.10-6.20 (1H, brs), 6.95 (1H, m), 7.10-7.15 (2H, m), 7.25 (1H, t)

### 16B. {3-[3-(4,4,5,5-Tetramethyl-[1,3,2]dioxaborolan-2-yl)-phenoxy]-propyl}-carbamic acid tert-butyl ester

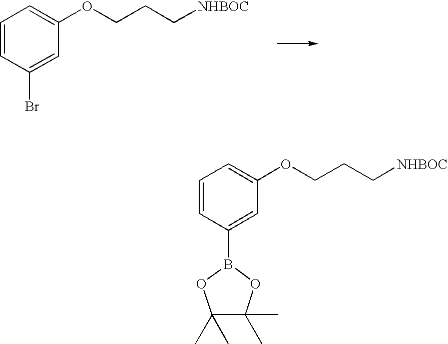

To tris(dibenzylideneacetone)dipalladium (0) (Pd₂ dba₃) (100 mg, 0.11 mmol) and tricyclohexylphosphine (76 mg, 0.27 mmol) was added dioxane (30 ml). The solution was degassed, and stirred at room temperature for 30 minutes. (Bis-pinacolato)diboron (1.44 g, 5.67 mmol), [3-(3-bromo-phenoxy)-propyl]-carbamic acid tert-butyl ester (1.80 g, 5.45 mmol) and potassium acetate (0.86 g, 8.76 mmol) were added, and the solution was heated at 80° C. for 16 h. After cooling to room temperature, the solution was poured into ethyl acetate (150 ml) and washed with water (50 ml) and brine (50 ml). The organic layer was dried, concentrated and purified by flash column chromatography (silica gel, 4:1 hexane:ethyl acetate) to yield the product (0.844g, 43% yield). 1H NMR (250 MHz, CDCl₃) 1.29 (9H, s), 1.37 (6H, s), 1.47 (6H, s), 1.99 (2H, ddt, J 6.2, 6.2, 6.2 Hz), 3.30-3.40 (2H, m), 3.98-4.07 (2H, m), 6.80-7.40 (4H, m)

### 16C. (3-{3-[9-(Tetrahydro-pyran-2-yl)-9H-purin-6-yl]-phenoxy}-propyl)-carbamic acid tert-butyl ester

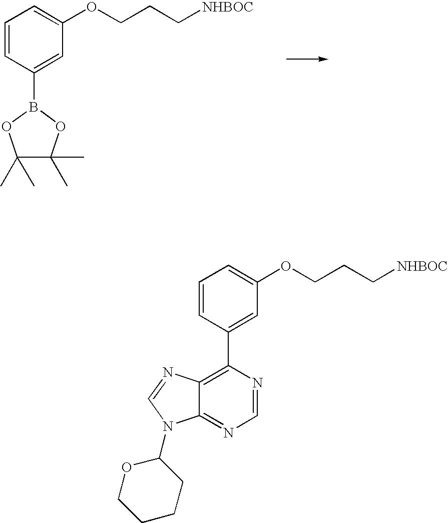

To a solution of {3-[3-(4,4,5,5-tetramethyl-[1,3,2]dioxaborolan-2-yl)-phenoxy]-propyl}-carbamic acid tert-butyl ester (0.252 mg, 0.68 mmol) in DME (7 ml) were added potassium carbonate (1 ml, 2M aqueous solution, 2 mmol), 6-chloro-9-(tetrahydro-pyran-2-yl)-9H-purine (157 mg, 0.65 mmol) and Pd(PPh₃)₄ (90 mg, 0.08 mmol). The solution was heated at reflux for 8 hours, then cooled to room temperature and poured into ethyl acetate (75 ml). The solution was washed with saturated NaHCO₃ (50 ml), brine (50 ml), then dried, concentrated and purified by flash column chromatography (SiO₂, 1:1 hexane:ethyl acetate) to yield the desired product. ¹H NMR (250 MHz, CDCl₃) 1.48 (9H, s), 1.60-2.30 (7H, m), 3.39 (2H, m), 3.84 (1H, dt, J 2.8, 11.0 Hz), 4.16-4.30 (3H, m), 5.00 (1H, brs), 5.88 (1H, dd, J 2.9, 9.8 Hz), 7.10 (1H, ddd, J1.0, 2.6, 8.2 Hz), 7.48 (1H, m), 8.30-8.40 (2H, m), 8.45 (1H, m), 9.03 (1H, s)

### 16D. 3-[3-(9H-Purin-6-yl)-phenoxy]-propylamine

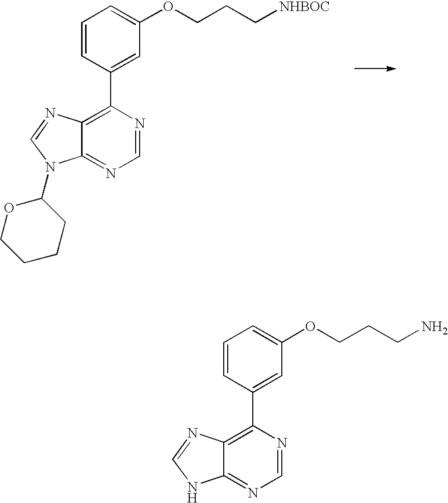

To a solution of (3-{3-[9-(tetrahydro-pyran-2-yl)-9H-purin-6-yl]-phenoxy}-propyl)-carbamic acid tert-butyl ester (77.5 mg, 0.17 mmol) in ethanol (1 ml) was added HCl (1 ml, 4M solution in dioxane, 4 mmol). The solution was stirred for 16 hours, and then concentrated under vacuum. The residue was dissolved in methanol and loaded onto an acidic resin SCX-2 cartridge, and washed with methanol (2×10 ml). Elution with 1M NH₃ in methanol gave the product (44 mg, 96% yield). LC/MS (LCT1) Rₜ 3.37 [M+H]⁺ 270

### Example 17

C-[1-(1H-Pyrazolo[3,4-d]pyrimidin-4-yl)-piperidin-4-yl]-methylamine

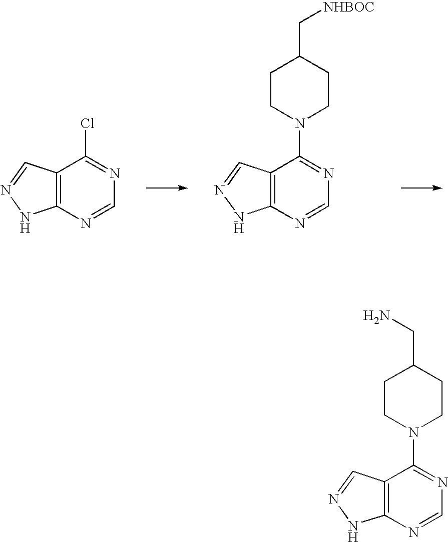

To a solution of 4-chloro-1H-pyrazolo[3,4-d]pyrimidine (J. Amer. Chem. Soc. 1957, 79, 6407-6413) (51 mg, 0.33 mmol) in ethanol (2 ml) was added triethylamine (100 μl, 0.72 mmol) and 4-(N-Boc-aminomethyl)piperidine (87 mg, 0.41 mmol). The solution was heated at 80° C. for 3 hours, and then cooled to room temperature. The solution was evaporated to dryness and the residue purified by recrystallisation (isopropanol) to yield the intermediate NH-BOC protected product (33 mg, 30% yield).

To the intermediate NH-BOC protected product (32 mg, 0.096 mmol) was added HCl (1 ml, 4M solution in dioxane, 4 mmol). The suspension was stirred at room temperature for 1 hour, and then diluted with diethyl ether (4 ml). The ethereal layer was discarded and the solid washed with a further portion of diethyl ether (2 ml). The ethereal layer was again discarded, and the resultant solid dried under high vacuum. The free base was liberated by dissolution of this material in methanol, loading onto an acidic resin SCX-2 cartridge, and elution from the cartridge with ammonia in methanol to give the title compound (21 mg, quantitative). LC/MS Rₜ 0.86 [M+H]⁺ 233

### Example 18

### C-[1-(7H-Pyrrolo[2,3-d]pyrimidin-4-yl)-piieridin-4-yl]-methylamine

### 18A. 6-Amino-5-(2,2-diethoxy-ethyl)-2-mercapto-pyrimidin-4-ol

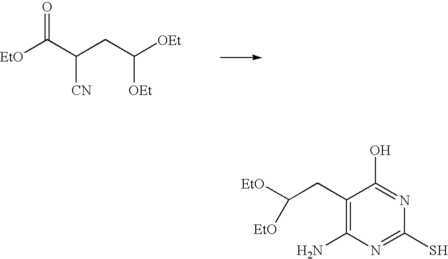

To ethanol (200 ml) was added sodium (2.05 g, 89 mmol) in small portions. The solution was stirred until complete dissolution of the sodium metal. 2-Cyano-4,4-diethoxy-butyric acid ethyl ester (J. Chem. Soc., 1960, 131-138) (9.292 g, 40.5 mmol) was then added as a solution in ethanol (50 ml), followed by addition of thiourea (3.08 g, 40.4 mmol). The solution was heated at 85° C. for 18 hours, and then cooled to room temperature. The solution was concentrated, and saturated aqueous ammonium chloride solution (150 ml) was added. The mixture was stirred at room temperature for 18 hours, after which time the solid was collected by filtration, and washed with water (20 ml) to yield the product (3.376 g, 36%).

### 18B. 6-Amino-5-(2,2-diethoxy-ethyl)-pyrimidin-4-ol

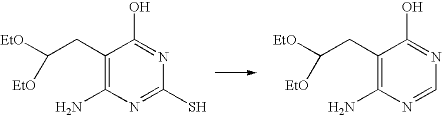

To a suspension of 6-amino-5-(2,2-diethoxy-ethyl)-2-mercapto-pyrimidin-4-ol (1.19 g, 4.6 mmol) in water (50 ml) was added Raney nickel (Aldrich Raney 2800 nickel, 4.8 ml). The mixture was heated at reflux for 1 hour, and then the hot solution was filtered through Celite®. The nickel residue was washed with further water (100 ml), and the washings were filtered through Celite. The aqueous filtrate was evaporated to dryness to yield the product (0.747 g, 71%).

### 18C. 7H-Pyrrolo[2,3-d]pyrimidin-4-ol

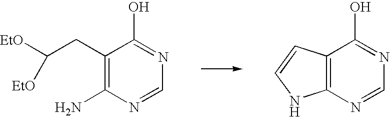

7H-Pyrrolo[2,3-d]pyrimidin-4-ol was prepared from 6-amino-5-(2,2-diethoxy-ethyl)-pyrimidin-4-ol by the method described in J. Chem. Soc., 1960, pp. 131-138.

### 18D. 4-Chloro-7H-pyrrolo[2,3-d]pyrimidine

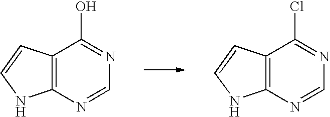

To 7H-pyrrolo[2,3-d]pyrimidin-4-ol (0.425 g, 3.14 mmol) was added phosphorus oxychloride (4 ml). The mixture was heated at reflux for 90 minutes and then cooled to room temperature. The solution was poured onto cracked ice, and extracted with chloroform (3×50 ml) and ethyl acetate (100 ml). The extracts were then dried and concentrated, and the residue obtained triturated with hot ethyl acetate (200 ml) to yield the desired product (0.204g, 42%).

### 18E. [1-(7H-Pyrrolo[2,3-d]pyrimidin-4-yl)-piperidin-4-ylmethyl]-carbamic acid tert-butyl ester

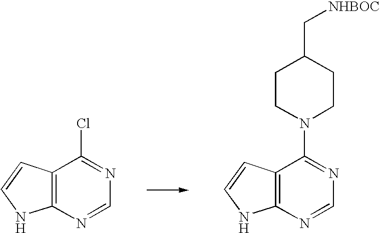

To a solution of 4-chloro-7H-pyrrolo[2,3-d]pyrimidine (67 mg, 0.44 mmol) in ethanol (1 ml) was added triethylamine (200 p1, 1.43 mmol) and 4-N-Boc-aminomethyl-piperidine (103 mg, 0.48 mmol). The solution was heated at 80° C. for 4 hours, and then cooled to room temperature. The precipitate was collected by filtration, recrystallised from ethanol-water (1:3) then dried under vacuum to yield the product (41 mg, 28%). LC/MS (LCT1) Rₜ 4.68 [M+H]⁺ 332

### 18F. C-[1-(7H-pyrrolo[2,3-d]pyrimidin-4-yl)-piperidin-4-yl]-methylamine

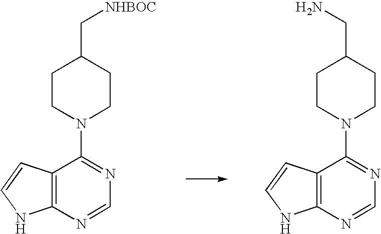

The product of Example 18E was deprotected by the method of Example 17 to give the title compound. LC/MS (LCT1) Rₜ 0.85 [M+H]⁺ 232

### Example 19

### C-Phenyl-C-[4-(9H-purin-6-yl)-phenyl]-methylamine

### 19A. 2-Methyl-propane-2-sulphinic acid 4-[9-(tetrahydro-pyran-2-yl)-9H-Purin-6-yl]-benzylideneamide

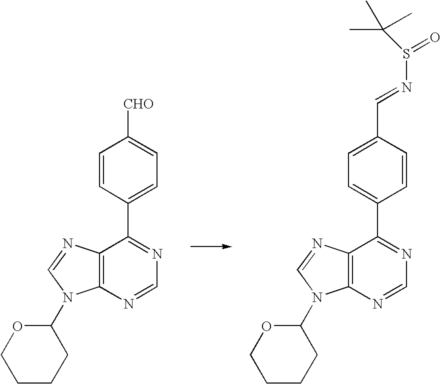

To a solution of racemic tert-butanesulphinamide (105 mg, 0.87 mmol) in dry dichloromethane (3.4 ml) was added pyridinium p-toluenesulphonate (6 mg, 0.025 mmol) and anhydrous magnesium sulphate (140 mg, 1.16 mmol) followed by the aldehyde of Example 6A (200 mg, 0.67 mmol). The mixture was stirred at room temperature under nitrogen for 48 hours (J. Am. Chem. Soc., 1997, 119, 9913). The reaction mixture was then filtered through a pad of Celite®, washed with dichloromethane and the solvent was evaporated in vacuo. The crude product was purified by flash silica column chromatography eluting with ethyl acetate/hexane (6:4) to afford the required compound as a white solid (124 mg, 0.30 mmol, 45%). LC/MS (LCT1) Rₜ 7.24 [M+H]⁺ 412.

### 19B. 2-Methyl-propane-2-sulphinic acid (phenyl-{4-[9-(tetrahydro-pyran-2-yl)-9H-purin-6-yl]-phenyl}-methyl)-amide

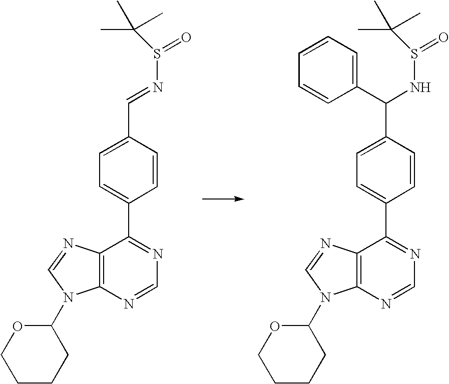

To a solution of the sulphinamide (37 mg, 0.09 mmol) in dry dichloromethane (1 ml) was added dropwise phenyl magnesium bromide 3M solution in diethyl ether (0.06 ml, 0.18 mmol), with stirring at −60° C. After stirring for 1 hour at −60° C. the temperature was increased slowly to 0° C. TLC analysis showed that the starting material had been consumed after 3 hours. The reaction mixture was quenched with saturated aqueous ammonium chloride (1 ml) and extracted with ethyl acetate. The combined organic layers were dried (MgSO₄) and concentrated in vacuo. The crude material was purified by flash silica column chromatography eluting with ethyl acetate/hexane (8:2) to afford the required compound (17 mg, 0.034 mmol, 38%). LC/MS (LCT1) Rₜ 7.14 [M+H]⁺ 490.

### 19C. C-Phenyl-C-[4-(9H-purin-6-yl)-phenyl]-methylamine

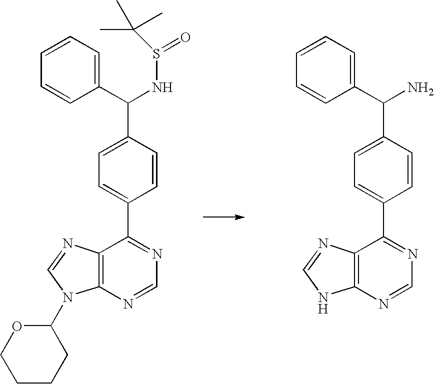

A solution of 2-methyl-propane-2-sulphinic acid (phenyl-{4-[9-(tetrahydro-pyran-2-yl)-9H-purin-6-yl]-phenyl}-methyl)-amide (16 mg, 0.033 mmol), ethanol (1.3 ml) and 1M aqueous HCl solution (1 ml) was stirred overnight at room temperature. The solvents were evaporated in vacuo and the crude material was passed through a basic resin NH₂ cartridge (2 g, 15 ml) eluting with methanol to afford the required compound (5.3 mg, 0.017 mmol, 53%). LC/MS (LCT1) Rₜ 4.19 [M+H]⁺ 302.

### Example 20

### 2-Phenyl-1-[4-(9H-purin-6-yl)-phenyl]-ethylamine

### 20A. 2-Methyl-propane-2-sulphinic acid (2-phenyl-1-{4-[9-(tetrahydro-pyran-2-yl)-9H-purin-6-yl]-phenyl}-ethyl)-amide

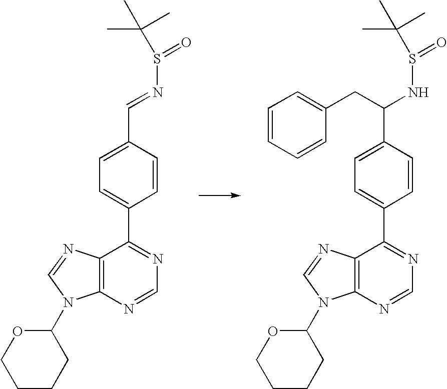

To a solution of the sulphinamide of Example 19A (38 mg, 0.09 mmol) in dry tetrahydrofuran (3 ml) was added dropwise benzyl magnesium chloride 2M solution in tetrahydrofuran (0.14 ml, 0.28 mmol), with stirring at room temperature. The solution was refluxed under nitrogen for 3 hours. The reaction mixture was cooled, quenched with saturated aqueous ammonium chloride (1 ml) and extracted with ethyl acetate. The combined organic layers were dried (MgSO₄) and concentrated in vacuo. The crude material was purified by flash silica column chromatography eluting with ethyl acetate/hexane (8:2) to afford the required compound (13 mg, 0.034 mmol, 29%). LC/MS (LCT1) Rₜ 7.34 [M+H]⁺ 504.

### 20B. 2-Phenyl-1-[4-(9H-purin-6-yl)-phenyl]-ethylamine

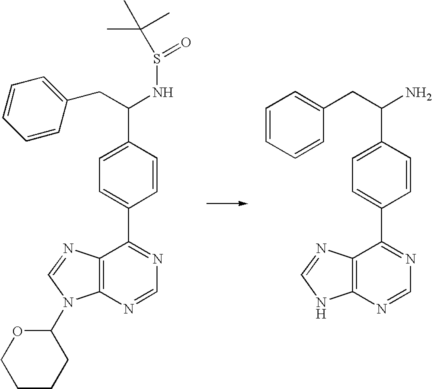

A solution of the product of Example 20A (2-methyl-propane-2-sulphinic acid (2-phenyl-1-{4-[9-(tetrahydro-pyran-2-yl)-9H-purin-6-yl]-phenyl}-ethyl)-amide) (13 mg, 0.026 mmol), methanol (0.5 ml) and HCl 4M solution in dioxane (0.04 ml) was stirred overnight at room temperature. The solvents were evaporated in vacuo and the crude material was passed through a basic resin NH₂ cartridge (2 g, 15 ml) eluting with methanol to afford the required compound (3.5 mg, 0.011 mmol, 43%). LC-MS (LCT1) Rₜ 4.37 [M+H]⁺ 316.

### Example 21

### 6-[4-(1-Amino-2-phenylethyl)piperidin-1-yl]-7,9-dihydropurin-8-one

### 21A. 4-(1-Hydroxy-2-phenylethyl)piperidine-1-carboxylic acid tert-butyl ester

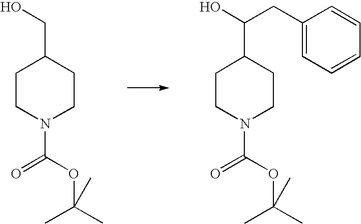

To a mixture of alcohol (0.503 g, 2.336 mmol), 4-methylmorpholine N-oxide (NMO) (356 mg, 3.037 mmol) and molecular sieves (4.0 g) in dichloromethane (23 ml) at 0° C. was added tetrapropylammonium perruthenate (TPAP) (41 mg, 0.117 mmol). After stirring for 2 hours at room temperature, the mixture was filtered through a pad of silica, washing with diethyl ether, and concentrated to give the crude aldehyde (not shown).

To a solution of the crude aldehyde in diethyl ether (20 ml) at 0° C. was added a solution of benzylmagnesium bromide (prepared from benzyl bromide (695 l, 5.840 mmol) and magnesium (153 mg, 6.307 mmol) in diethyl ether (12 ml)). After stirring at room temperature for 15 hours, saturated aqueous ammonium chloride (150 ml) was added, the phases were separated and the aqueous phase extracted with diethyl ether (50 ml). The organic phases were combined, dried (magnesium sulphate) and concentrated, and the resulting crude product was purified by silica column chromatography (60% diethyl ether/hexane) to give the title alcohol as a clear oil (256 mg, 36%). LC/MS: (LCT1) Rₜ 7.11 [M+Na]+328.

### 21B. 4-Phenylacetylpiperidine-1-carboxylic acid tert-butyl ester

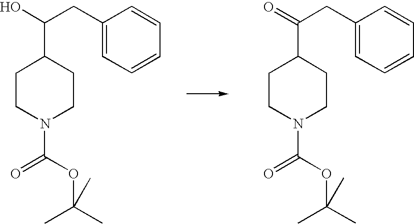

To a mixture of the alcohol of Example 21A (0.241 g, 0.789 mmol), NMO (129 mg, 1.105 mmol) and molecular sieves (1.5 g) in dichloromethane (8 ml) at 0° C. was added TPAP (14 mg, 0.039 mmol). After stirring for 15 hours at room temperature, the mixture was filtered through a pad of silica, washing with diethyl ether, and concentrated. The crude material was purified by silica column chromatography (60% diethyl ether/hexane) to give the title ketone as a clear oil (101 mg, 42%). LC/MS (LCT1): Rₜ 6.93 [M+Na]⁺ 326.

### 21C. 6-(4-Phenylacetylpiperidin-1-yl)-7,9-dihydropurin-8-one

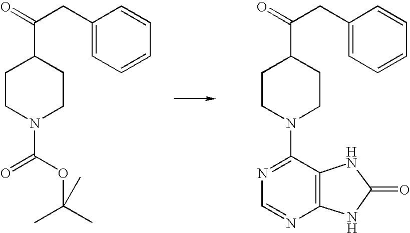

To a solution of the ketone of Example 21B (101 mg, 0.33 mmol) in diethyl ether (3 ml) was added 1 M HCl in diethyl ether (3 ml, 3 mmol). After 3 hours, methanol (2 ml) was added. After 2 days the suspension was concentrated. Solid phase extraction on SCX-II acidic resin, eluting with MeOH then 1 M NH₃ in MeOH, gave the deprotected piperidine (54 mg, 0.266 mmol).

To a solution of the deprotected piperidine (54 mg, 0.266 mmol) and 6-chloro-7,9-dihydropurin-8-one (45 mg, 0.266 mmol) in n-butanol (2.7 ml) was added triethylamine (185 l, 1.328 mmol). After refluxing for 24 hours, the solution was cooled, concentrated and the resulting solid triturated with methanol (5 ml) to give the title ketone as a white solid (18 mg, 20%). LC/MS (LCT1): Rₜ 5.84 [M+H]⁺ 338.

### 21D. 6-[4-(1-Amino-2-Phenylethyl)piperidin-1-yl]-7,9-dihydropurin-8-one

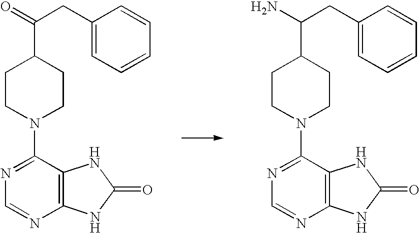

To a solution of the purinone of Example 21C (0.015 g, 0.0445 mmol) in methanol (1 ml) was added ammonium acetate (41 mg, 0.5335 mmol) and sodium cyanoborohydride (11 mg, 0.1778 mmol). After refluxing for 2 days, the suspension was cooled, then purified by solid phase extraction on SCX-II acidic resin, eluting with MeOH then 1M NH₃ in MeOH, which gave the title amine as a 9:1 mixture with starting material. The above reaction sequence was repeated to give the title amine as a white solid (15 mg, 100%). LC/MS (LCT1): Rₜ 4.15 [M+H]⁺ 339.

### Example 22

### 6-(4-[4-(4-Chlorophenyl)-piperidin-4-yl)-phenyl)-9H-purine

### 22A. 4-(4-Bromo-phenyl)-4-(4-chloro-phenyl)-piperidine

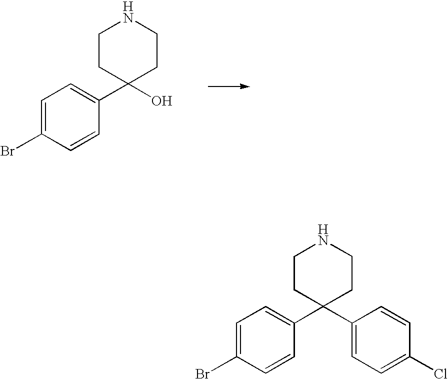

A suspension of 4-(4-bromo-phenyl)-piperidin-4-ol (4.02 g, 15.7 mmol) in chlorobenzene (30 ml) was added dropwise to a suspension of aluminium chloride (7.32 g, 54.9 mmol) in chlorobenzene (10 ml) at 0° C. The reaction mixture was stirred at 0° C. for 2 hours, quenched by addition of ice then methyl t-butyl ether added. After stirring for 1 hour the precipitate was collected by filtration washed with water, methyl t-butyl ether and water to afford the title compound (5.59 g, 92% yield). LC/MS: (PS-B3) Rₜ 3.57 [M+H]⁺ 350, 352.

### 22B. 4-(4-Bromophenyl)-4-(4-chlorophenyl)-piperidine-1-carboxylic acid tert-butyl ester

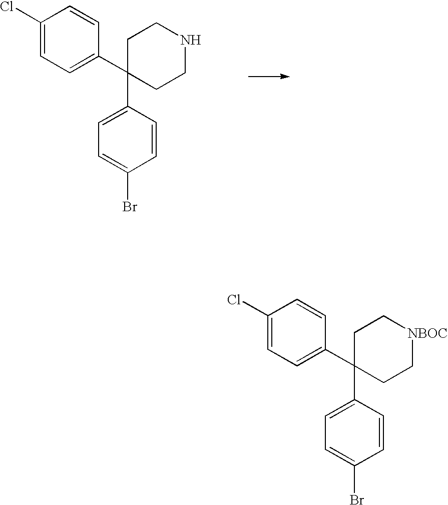

A solution of the 4-(4-bromophenyl)-4-(4-chlorophenyl)-piperidine hydrochloride of Example 22A (1.02 g, 2.64 mmol), triethylamine (2.8 ml, 20 mmol) and di-tert-butyldicarbonate (0.60 g, 2.75 mmol) in dichloromethane (50 ml) was stirred at room temperature for 24 hours. The solution was rinsed with 1 M citric acid (50 ml), dried (Na₂SO₄), filtered and concentrated to give a white solid (1.15 g, 97%). ¹H NMR (250 mHz, CDCl₃) 1.47 (9H, s), 2.31-2.35 (4H, m), 3.46-3.52 (4H, m), 7.10-7.20 (4H, m), 7.28 (2H, d, J=6 Hz), 7.44 (2H, d, J=6 Hz).

### 22C. 4-(4-(4-Chlorophenyl)-piperidin-4-yl)-phenylboronic acid

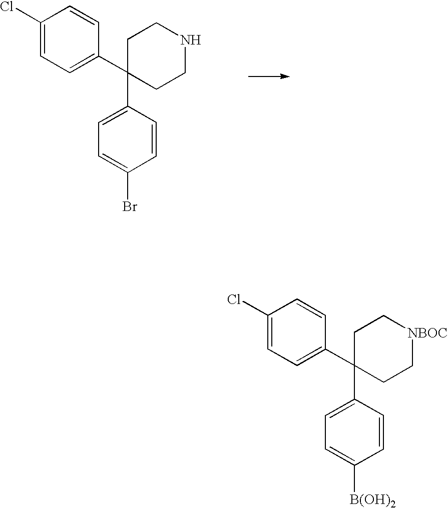

A solution of the 4-(4-bromophenyl)-4-(4-chlorophenyl)-piperidine-1-carboxylic acid tert-butyl ester of Example 22B (0.50 g, 1.11 mmol) and triisopropylborate (0.31 ml, 1.33 mmol) in dry THF (6 ml) was stirred at −78° C. under nitrogen. A solution of n-butyllithium (2M in pentane, 0.67 ml, 1.33 mmol) was added dropwise. The deep red solution was stirred at −78° C. for 30 minutes, becoming pale yellow, then warmed to room temperature and quenched with 1 M HCl (aq) (2 ml). The mixture was stirred for 5 minutes then diluted with H₂O (25 ml) and extracted with EtOAc (25 ml). The extract was dried (Na₂SO₄), filtered and concentrated to give a sticky yellow foam. Crystallisation from acetonitrile gave a white solid (0.188 g, 41%).

### 22D. 6-(4-(4-(4-Chlorophenyl)-piperidin-4-yl)-phenyl)-9-(tetrahydropyran-2-yl)-9H-purine

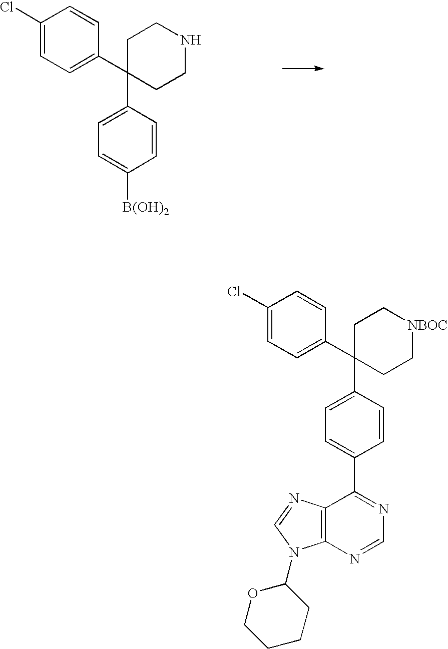

A solution of the boronic acid of Example 22C (0.083 g, 0.2 mmol), 6-chloro-9-(tetrahydropyran-2-yl)-9H-purine (0.050 g, 0.21 mmol), 2M K₂CO₃ (aq) (0.20 ml, 0.40 mmol) and Pd(PPh₃)₄ (0.02 g, 7 mol %) in 1,2-dimethoxyethane (3 ml) was degassed and flushed with nitrogen. The solution was stirred at 85° C. for 16 hours. The solution was partitioned between EtOAc (15 ml) and H₂O (15 ml). The organic layer was dried (Na₂SO₄), filtered and concentrated. Preparative t.l.c., eluting with 50% EtOAc/50% hexane, gave the title product (0.030g, 26%). LC/MS: (LCT1) Rₜ 8.34 [M+H-THP-tBu]⁺ 434, 436.

### 22E. 6-(4-[4-(4-Chlorophenyl)-piperidin-4-yl)-phenyl)-9H-purine

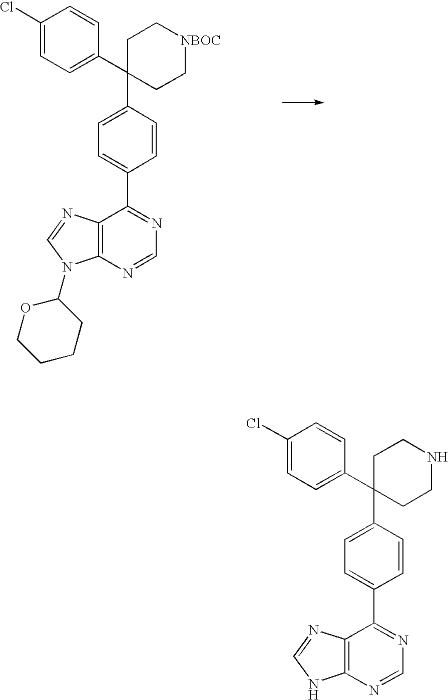

A solution of the protected purine of Example 22D in EtOH (4 ml) with 1 M HCl (aq) (2 ml) was stirred at room temperature for 24 hours. Concentrated HCl (3 drops) was added and the mixture was stirred at room temperature for 24 hours, then at 80° C. for 5 hours. The solution was absorbed onto a 5g SCX-II acidic resin cartridge and eluted with MeOH, then 1 M NH₃/MeOH. The basic eluant was concentrated. Trituration and rinsing with diethyl ether gave the product as an off-white solid (0.014g, 69%). LC/MS: (LCT1) Rₜ 5.00 [M+H]⁺390, 392.

### Example 23

### 4-{4-[4-(4-Chloro-phenyl)-piperidin-4-yl]-phenyl}-7H-pyrrolo[2,3-d]pyrimidine

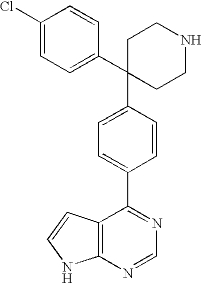

By following a procedure analogous to the method set out in Example 22, the title compound was prepared. LC/MS (LCT1) Rₜ 4.48 (ESI) m/z 389 [M+H]⁺

### Example 24

### C-Phenyl-C-[1-(7H-pyrrolo[2,3-d]pyrimidin-4-yl)-piperidin-4-yl]-methylamine

### 24A. 4-(4-Chlorobenzoyl)piperidine-1-carboxylic acid benzyl ester

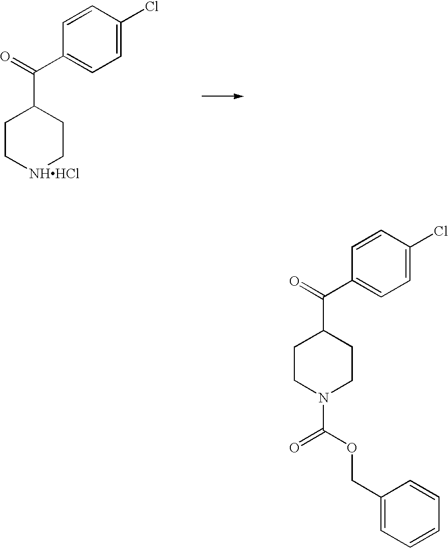

To a mixture of (4-chlorophenyl)piperidin-4-ylmethanone hydrochloride (0.752 g, 2.890 mmol) and triethylamine (1.21 ml, 8.670 mmol) in DCM (20 ml) at 0° C. was added benzyl chloroformate (0.495 ml, 3.468 mmol). After 18 hours at room temperature, the mixture was washed with saturated aqueous sodium bicarbonate (25 ml), then brine (25 ml) before being dried over sodium sulfate and concentrated. The crude material was purified by silica column chromatography (ethyl acetate) to give the ketone as an oil (0.934g, 100%). LC/MS: (LCT1) Rₜ 7.47 [M+H]⁺ 357.

### 24B. 4-[Amino-(4-chlorophenyl)methyl]piperidine-1-carboxylic acid benzyl ester

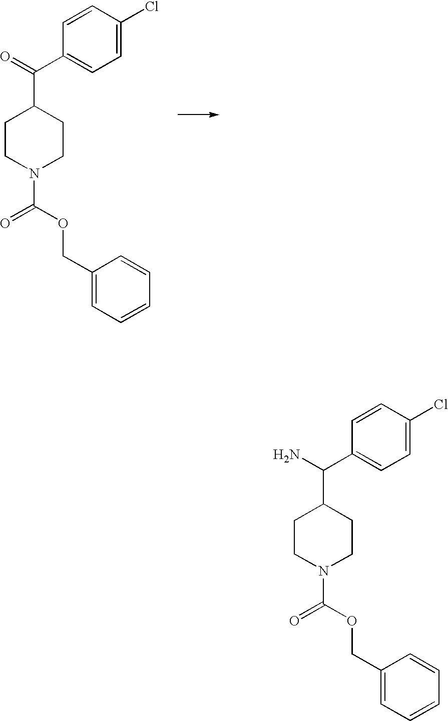

To a mixture of 4-(4-chlorobenzoyl)piperidine-1-carboxylic acid benzyl ester (0.630 g, 1.948 mmol) and ammonium acetate (1.802 g, 23.377 mmol) in methanol (19.5 ml) at room temperature was added sodium cyanoborohydride (0.490 g, 7.792 mmol). After refluxing for 20 hours the mixture was cooled, concentrated and stirred with 1 M sodium hydroxide (50 ml). The aqueous phase was extracted with diethyl ether (3×50 ml), with the organic layers being combined, dried over sodium sulphate and concentrated to give the amine as an oil (0.611 g, 97%). LC/MS (LCT1): Rₜ 10.67 [M+H]⁺ 358.

### 24C. 4-[tert-Butoxycarbonylamino(4-chlorophenyl)methyl]piperidine-1-carboxylic acid benzyl ester

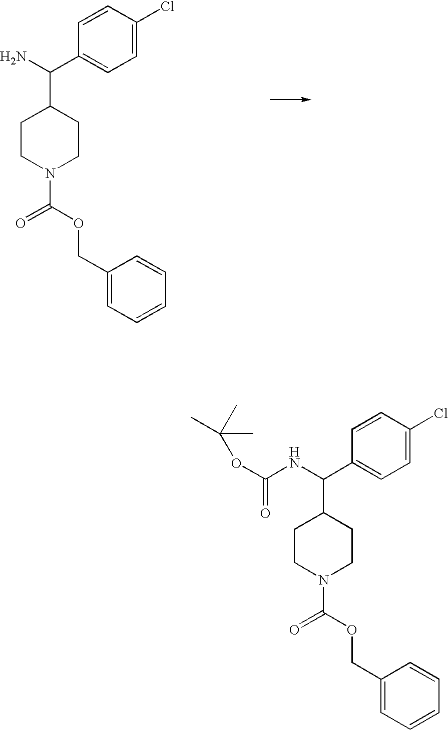

To a solution of 4-[amino-(4-chlorophenyl)methyl]piperidine-1-carboxylic acid benzyl ester (0.611 g, 1.883 mmol) and di-tert-butyl dicarbonate (0.493 g, 2.260 mmol) in acetonitrile (19 ml) at room temperature was added triethylamine (0.788 ml, 5.650 mmol). After 24 hours, the mixture was concentrated, redissolved in ethyl acetate (50 ml) and the organic phase washed with saturated aqueous sodium bicarbonate (50 ml) then brine (50 ml). The organic phase was dried over magnesium sulphate, concentrated and the resulting crude product purified by silica column chromatography (60% diethyl ether in hexanes) to give the protected amine as an oil (0.600g, 69%). LC/MS (LCT1): Rₜ 7.79 [M+H]⁺ 458.

### 24D. Phenylpiperidin-4-ylmethyl carbamic acid tert-butyl ester

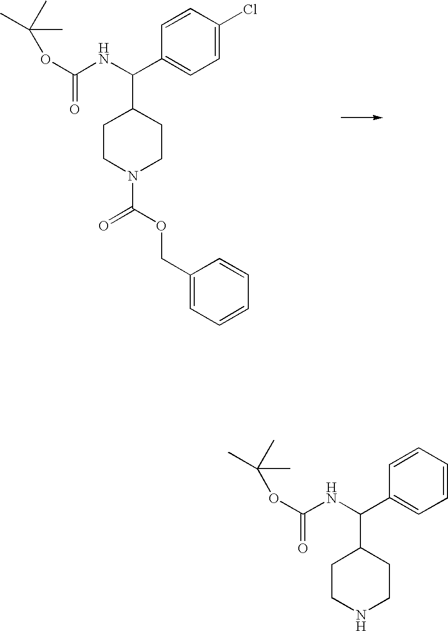

A solution of 4-[tert-butoxycarbonylamino(4-chlorophenyl)methyl]piperidine-1-carboxylic acid benzyl ester (0.217 g, 0.473 mmol) in ethanol (20 ml) was stirred under 1 atmosphere hydrogen pressure over 5% palladium on carbon (40 mg) at room temperature for 1 hour. The reaction mixture was filtered through a pad of celite and the filtrate concentrated to give an oil (0.136 g, 100%). LC/MS (LCT1): Rₜ 4.15 [M+H]⁺ 290.

### 24E. {Phenyl-[1-(7H-pyrrolo[2,3-d]pyrimidin-4-yl)piperidin-4-yl]methyl}carbamic acid tert-butyl ester

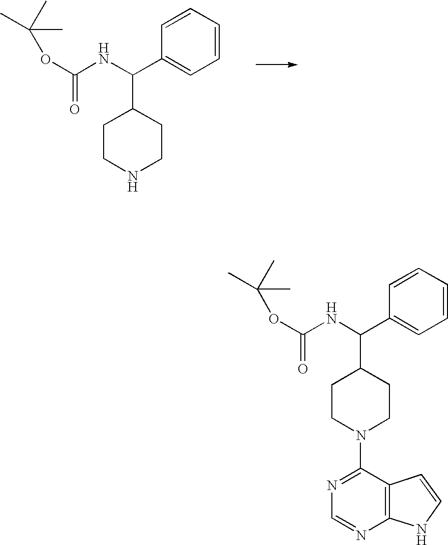

A solution of phenylpiperidin-4-ylmethyl carbamic acid tert-butyl ester (0.070 g, 0.216 mmol), 4-chloro-7H-pyrrolo[2,3-d]pyrimidine (0.033 g, 0.216 mmol) and triethylamine (0.15 ml, 1.078 mmol) in n-butanol (2 ml) was heated at 100° C. for 2 days. The crude mixture was concentrated and purified by silica column chromatography (10% methanol in DCM) to give an oil (52 mg, 59%). ¹H NMR (MeOD) 1.20-1.60 (2H, m), 1.43 (9H, s), 1.85-2.15 (2H, m), 2.98-3.16 (2H, m), 4.32-4.36 (1H, m), 4.67-4.88 (2H, m), 6.59-6.60 (1H, m), 7.11-7.13 (1H, m), 8.12 (1H, s).

### 24F. C-Phenyl-C-[1-(7H-pyrrolo[2,3-d]pyrimidin-4-yl)piperidin-4-yl]methylamine

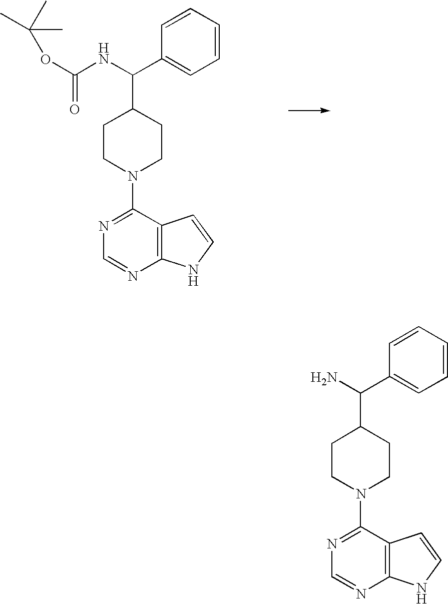

To a solution of {phenyl-[1-(7H-pyrrolo[2,3-d]pyrimidin-4-yl)piperidin-4-yl]methyl}carbamic acid tert-butyl ester (0.050 g, 0.123 mmol) in methanol (3 ml) at room temperature was added 2M hydrochloric acid (3 ml). After 13 hours the mixture was evaporated to dryness. Solid phase extraction on SCX-II acidic resin, eluting with MeOH then 1M NH₃ in MeOH, gave the deprotected amine as a white solid (0.035 g, 92%). LC/MS (LCT1): Rₜ 2.70 [M+H]⁺ 307.

### Example 25

C-4-Chlorophenyl-C-[1-(7H-pyrrolo[2,3-d]pyrimidin-4-yl)-piperidin-4-yl]-methylamine

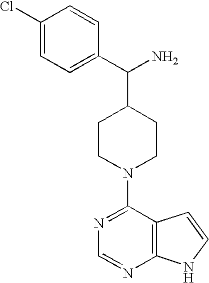

### 25A. 4-(4-Chlorobenzoyl)piperidine-1-carboxylic acid tert-butyl ester

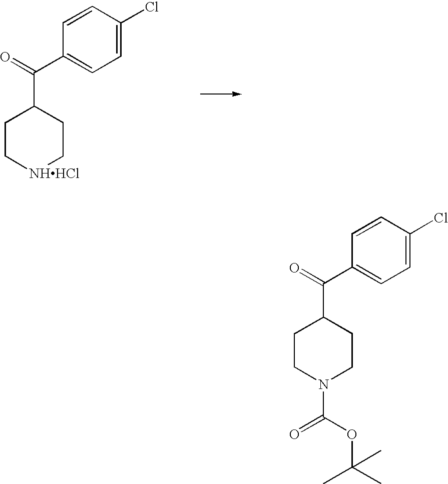

To a mixture of (4-chlorophenyl)piperidin-4-ylmethanone hydrochloride (0.996 g, 3.828 mmol) and triethylamine (2.7 ml, 19.142 mmol) in acetonitrile (15 ml) at room temperature was added di-tert-butyl dicarbonate (1.003 g, 4.594 mmol). After 16 hours at room temperature, the mixture was evaporated to dryness and then partitioned between ethyl acetate (50 ml) and 1 M hydrochloric acid (20 ml). The organic phase was separated and washed successively with saturated aqueous sodium bicarbonate (20 ml), then brine (20 ml), before being dried over magnesium sulfate and concentrated to dryness. The crude material was purified by silica column chromatography (60% diethyl ether in hexanes) to give the ketone as an oil (1.116 g, 90%). LC/MS: (LCT1) Rₜ 7.42 [M+H]⁺ 323.

### 25B. 4-[Amino-(4-chlorophenyl)methyl]piperidine-1-carboxylic acid tert-butyl ester

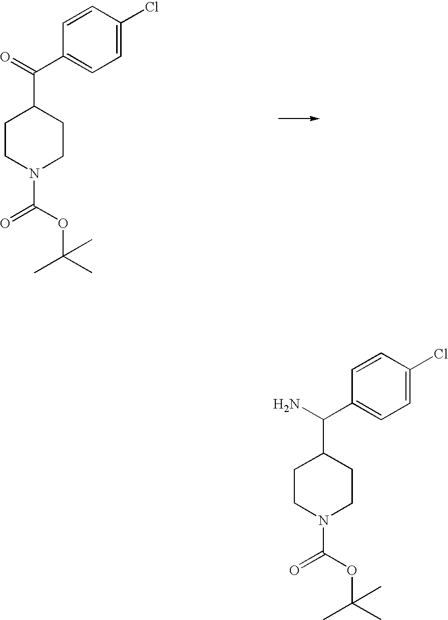

To a mixture of 4-(4-chlorobenzoyl)piperidine-1-carboxylic acid tert-butyl ester (1.116 g, 3.446 mmol) and ammonium acetate (3.188 g, 41.358 mmol) in methanol (34 ml) at room temperature was added sodium cyanoborohydride (0.866 g, 13.786 mmol). After refluxing for 20 hours, the mixture was cooled, concentrated and stirred with 1 M sodium hydroxide (100 ml). The aqueous phase was extracted with diethyl ether (3×75 ml), with the organic layers being combined, dried over sodium sulfate and concentrated to dryness. The crude material was purified by silica column chromatography (15% methanol in DCM) to give the amine as an oil (0.913 g, 82%). LC/MS (LCT1): Rₜ 5.56 [M-Boc-NH₂]+208.

### 25C. C-(4-Chlorophenyl)-C-piperidin-4-ylmethylamine hydrochloride

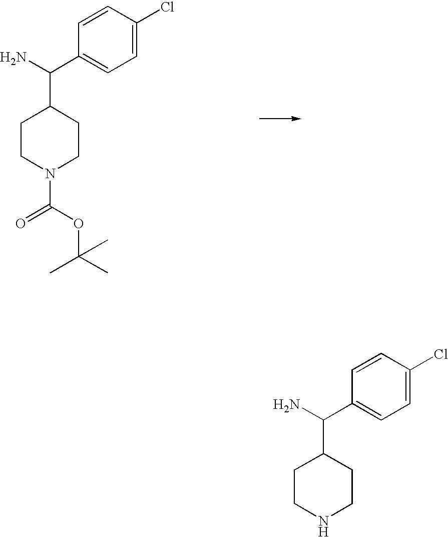

To a solution of 4-[amino-(4-chlorophenyl)methyl]piperidine-1-carboxylic acid tert-butyl ester (0.192 g, 0.591 mmol) in methanol (6 ml) at room temperature was added 2M hydrochloric acid (6 ml). After stirring for 16 hours the solution was evaporated to dryness to give the amine salt as a white foam (0.174g, 99%). ¹H NMR (MeOD) 1.40-1.82 (2H, m), 2.22-2.50 (2H, m), 2.90-3.17 (2H, m), 3.35-3.61 (2H, m), 4.22 (1H, d, 9.5 Hz), 7.53-7.61 (4H, m).

### 25D.C.-(4-Chlorophenyl)-C-[1-(7H-pyrrolo[2,3-d]pyrimidin-4-yl)piperidin-4-yl]methylamine

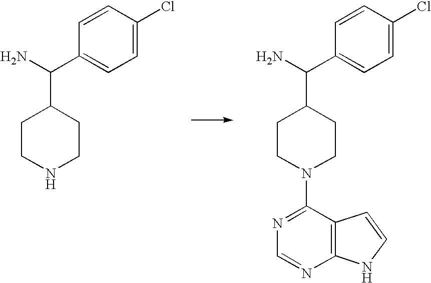

A solution of C-(4-chlorophenyl)-C-piperidin-4-ylmethylamine hydrochloride (0.050 g, 0.168 mmol), 4-chloro-7H-pyrrolo[2,3-d]pyrimidine (0.026 g, 0.168 mmol) and triethylamine (0.117 ml, 0.840 mmol) in n-butanol (1.7 ml) was heated at 100° C. for 2 days. The crude mixture was concentrated, passed through an —NH₂ Isolute column (2g), concentrated again and purified by silica column chromatography (15% methanol in DCM) to give a off white solid (30 mg, 52%). LC/MS (LCT1): Rₜ 3.35 [M+H]⁺ 341.

### Example 26

### C-(4-Chloro-phenyl)-C-[1-(9H-purin-6-yl)-piperidin-4-yl]-methylamine

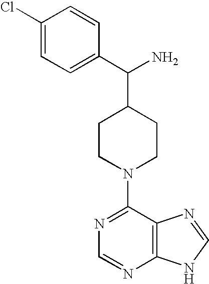

The title compound was prepared by reaction of C-(4-chloro-phenyl)-C-piperidin-4-yl-methylamine (Example 25C) and 6-chloropurine in n-butanol at 10° C. using the method described in Example 25D. LC/MS: (LCT1) Rₜ 4.13 [M+H]⁺ 342.

### Example 27

### 4-{4-[4-(4-Chloro-phenyl)-piperidin-4-yl]-phenyl}-1H-pyrrolo[2,3-b]pyridine

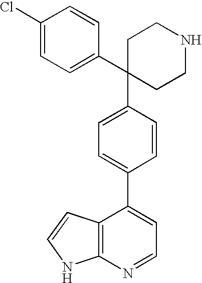

By following a procedure analogous to the method set out in Example 22, the title compound was prepared. LC/MS: (LCT1) Rₜ 4.34 [M+H]⁺ 388.

### Example 28

### C-(4-Chloro-phenyl)-C-[4-(9H-purin-6-yl)-phenyl]-methylamine

### 28A. (4-Bromo-phenyl)-(4-chloro-phenyl)-methanol

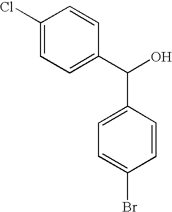

To a cooled (ice bath) solution of 4-bromobenzaldehyde (6.90 g, 37 mmol) in THF (20 ml) was added dropwise 4-chlorophenylmagnesium bromide (40 ml, 1 M solution in diethyl ether, 40 mmol). The solution was stirred for 50 minutes, and then saturated ammonium chloride (200 ml) was added, followed by ethyl acetate (250 ml). The layers were separated, and the organic fraction was washed with water (100 ml), then dried, concentrated and purified by flash column chromatography (6:1 hexane:ethyl acetate) to yield the desired product (4.47 g, 41% yield). ¹H NMR (250 MHz, d6-dmso) 3.50 (1H, br s), 5.71 (1H, s), 7.33 (4H, d, J=8.44 Hz), 7.38 (2H, s), 7.51 (2H, d, J=8.46 Hz)

### 28B. 2-[(4-Bromo-phenyl)-(4-chloro-phenyl)-methyl]-isoindole-1,3-dione

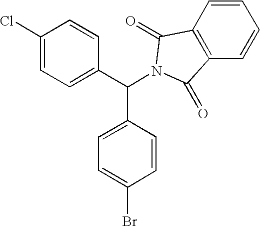

To a solution of (4-Bromo-phenyl)-(4-chloro-phenyl)-methanol (2.30 g, 7.73 mmol), triphenylphosphine (3.42 g, 13.03 mmol) and phthalimide (1.91 g, 12.98 mmol) in THF (60 ml) was added dropwise diisopropylazodicarboxylate (2.40 ml, 12.19 mmol). The solution was stirred for 18 hours, and was then poured into diethyl ether (250 ml). The solution was washed with saturated sodium bicarbonate (2 times 100 ml) and brine (50 ml). The organic fraction was then dried, concentrated and purified by flash column chromatography (6:1 hexane:ethyl acetate) to yield the desired product (0.698 g, 21% yield). LC/MS: (LCT1) Rₜ 8.21 [M+H]⁺ 426.

### 28C. 2-{(4-Chloro-phenyl)-[4-(4,4,5,5-tetramethyl-[1,3,2]dioxaborolan-2-yl)-phenyl]-methyl}-isoindole-1,3-dione

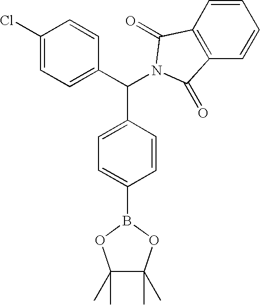

To Pd₂ dba₃ (13 mg, 0.014 mmol) and tricyclohexylphosphine (20 mg, 0.07 mmol) was added dioxane (6 ml). The solution was degassed, and stirred at room temperature for 30 minutes. Bis(pinacolato)diboron (0.256 g, 1 mmol), 2-[(4-Bromo-phenyl)-(4-chloro-phenyl)-methyl]-isoindole-1,3-dione (0.424 g, 1 mmol) and potassium acetate (0.164 g, 1.67 mmol) were then added, and the solution heated at 80° C. for 16 hours. After cooling to room temperature, the solution was poured into ethyl acetate (10 ml) and washed with water (50 ml) and brine (50 ml). The organic layer was dried, concentrated and purified by flash column chromatography (SiO₂, 6:1 hexane:ethyl acetate) to yield the desired product (0.142 g, 30% yield). LC/MS: (LCT1) Rₜ 8.55 [M+Na]⁺ 497.

### 28D. 2-((4-Chloro-phenyl)-{4-[9-(tetrahydro-pyran-2-yl)-9H-purin-6-yl]-phenyl}-methyl)-isoindole-1,3-dione

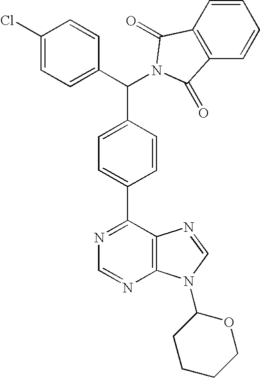

To a solution of 6-chloro-9-(tetrahydro-pyran-2-yl)-9H-purine (0.105 g, 0.44 mmol) and 2-{(4-chloro-phenyl)-[4-(4,4,5,5-tetramethyl-[1,3,2]dioxaborolan-2-yl)-phenyl]-methyl}-isoindole-1,3-dione (0.211 mg, 0.44 mmol) in DME (2 ml) was added PdCl₂(PPh₃)₂. 1 M K₂CO₃ (1 ml) was then added and the solution was heated at 80° C. for 18 hours. The mixture was then poured into chloroform/water (100 ml/50 ml), and the layers separated. The product was extracted with chloroform (100 ml) and the combined organic extracts were dried (Na₂SO₄), concentrated, then purified by flash column chromatography (1:1 hexane:ethyl acetate to 1:3 hexane:ethyl acetate) to yield the desired product (0.101 g, 42% yield).

1H NMR (250 MHz, CDCl₃) 1.60-2.30 (6H, m), 3.82 (1H, dt, J 2.76, 10.99 Hz), 4.15-4.26 (1H, m), 5.85 (1H, dd, J 3.1, 9.8 Hz), 6.77 (1H, s), 7.30-7.41 (4H, m), 7.55 (2H, d, J=8.38 Hz), 7.74 (2H, dd, J 3.04, 5,37 Hz), 7.86 (2H, dd, J 3.1, 5.61 Hz), 8.33 (1H, s), 8.76 (2H, d, J=8.46 Hz), 9.01 (1H, s)

### 28E. C-(4-Chloro-phenyl)-C-{4-[9-(tetrahydro-pyran-2-yl)-9H-purin-6-yl]-phenyl}-methylamine

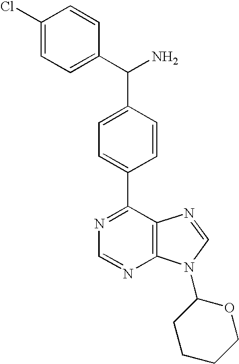

To a solution of 2-((4-chloro-phenyl)-{4-[9-(tetrahydro-pyran-2-yl)-9H-purin-6-yl]-phenyl}-methyl)-isoindole-1,3-dione (0.099 g, 0.18 mmol) in ethanol (6 ml) was added hydrazine hydrate (1 ml). The solution was stirred for 48 hours, then the precipitate was removed by filtration and the filtrate concentrated. The residue obtained was dissolved in methanol, loaded onto an SCX-2 cartridge (2g), washed with methanol (3 times 5 ml), then eluted with 2 M ammonia in methanol (3 times 5 ml). The product obtained was carried forward without further purification.

### 28F. Preparation of C-(4-Chloro-Phenyl)-C-[4-(9H-purin-6-yl)-phenyl]-methylamine

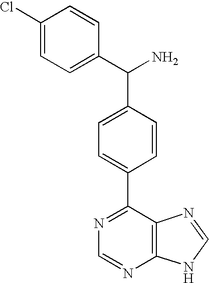

To a solution of C-(4-chloro-phenyl)-C-{4-[9-(tetrahydro-pyran-2-yl)-9H-purin-6-yl]-phenyl}-methylamine (carried forward from previous) in methanol (2 ml) was added 4M HCl in dioxane (2 ml). The mixture was stirred for 18 hours, and then concentrated. The residue was dissolved in methanol and loaded onto a SCX-2 cartridge (2g) and washed with methanol (3 times 5 ml), then product was eluted with 2M ammonia in methanol (3 times 5 ml). The solution was concentrated to yield the desired product (0.044g, 73% over 2 steps). LC/MS: (LCT1) Rₜ 4.48 [M+H]⁺ 336.

### Example 29

### C-(4-Chlorophenyl)-C-[1-(1H-pyrrolo[2,3-b]pyridin-4-yl)piperidin-4-yl]methylamine

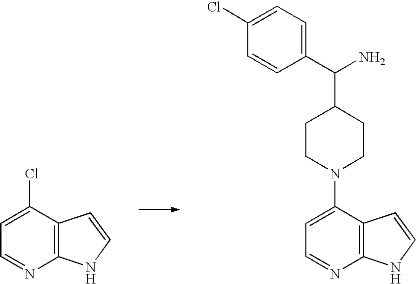

The title compound was prepared using the methods described in Example 25. LC-MS (LCT1) m/z 340 [M+H⁺], Rₜ 2.88 min.

### Example 30

{2-(4-Chloro-phenyl)-2-[4-(1H-pyrrolo[2,3-b]pyridin-4-yl)-phenyl]-ethyl}-methyl-amine

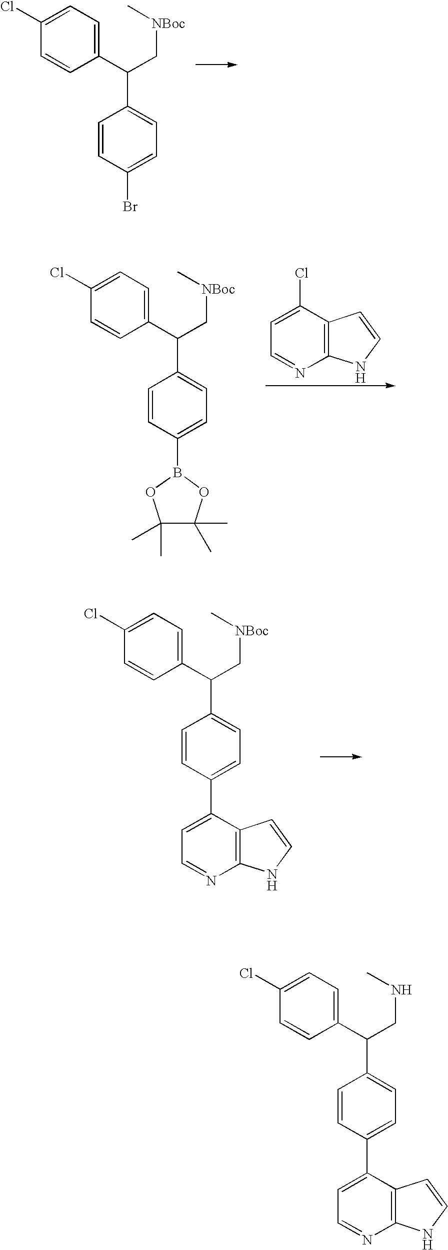

### 30A. {2-(4-Chloro-phenyl)-2-[4-(4,4,5,5-tetramethyl-[1,3,2]dioxaborolan-2-yl)-phenyl]-ethyl}-methyl-carbamic acid tert-butyl ester

Potassium acetate (218 mg, 2.2 mmol) was added to a degassed solution of [2-(4-bromo-phenyl)-2-(4-chloro-phenyl)-ethyl]-methyl-carbamic acid tert-butyl ester (550 mg, 1.30 mmol) and bis(pinacolato)diboron (338 mg, 1.32 mmol) in dry dioxane (8 ml) at room temperature. This solution was further degassed and flushed with nitrogen (2 cycles). Tricyclohexylphosphine (28 mg, 0.098 mmol) and tris(dibenzylideneacetone)dipalladium (0) (17.6 mg, 0.019 mmol) were added to the reaction mixture. The suspension was further degassed and stirred for 19 hours at 80° C. under nitrogen. After cooling to room temperature, the reaction mixture was partioned between ethyl acetate (50 ml) and water (50 ml). The organic layer was washed with water (2×30 ml), brine (50 ml), dried (Mg₂SO₄), filtered and concentrated. Flash column chromatography on silica, eluting with 15% ethyl acetate in hexane, gave {2-(4-chloro-phenyl)-2-[4-(4,4,5,5-tetramethyl-[1,3,2]dioxaborolan-2-yl)-phenyl]-ethyl}-methyl-carbamic acid tert-butyl ester (131 mg, 0.28 mmol, 21%). LC-MS (LCT2) m/z 494 [M+Na⁺], Rₜ 9.59 min.

### 30B. {2-(4-Chloro-phenyl)-2-[4-(1H-pyrrolo[2,3-b]pyridin-4-yl)-phenyl]-ethyl}-methyl-carbamic acid tert-butyl ester

A degassed mixture of {2-(4-chloro-phenyl)-2-[4-(4,4,5,5-tetramethyl-[1,3,2]dioxaborolan-2-yl)-phenyl]-ethyl}-methyl-carbamic acid tert-butyl ester (100 mg, 0.21 mmol), 4-chloro-1H-pyrrolo[2,3-b]pyridine (45 mg, 0.29 mmol), 2M aqueous solution of potassium carbonate (0.38 ml, 0.74 mmol), dioxane (4 ml) and Bedford's palladacycle catalyst (Bedford et al, Chem. Commun. 2001, 1540-1541) (13.5 mg, 0.016 mmol) was heated at 100° C. under nitrogen for 17 hours. The solution was cooled and partitioned between dichloromethane (40 ml) and water (40 ml). The aqueous layer was further extracted with dichloromethane (40 ml). The combined organic layers were dried (Na₂SO₄), filtered and concentrated. Flash column chromatography on silica, eluting with 50% ethyl acetate in hexane, gave {2-(4-chloro-phenyl)-2-[4-(1H-pyrrolo[2,3-b]pyridin-4-yl)-phenyl]-ethyl}-methyl-carbamic acid tert-butyl ester (49 mg, 0.11 mmol, 51%). LC-MS (LCT2) m/z 462 [M+H⁺], Rₜ 8.65 min.

### 30C. {2-(4-Chloro-phenyl)-2-[4-(1H-pyrrolo[2,3-b]pyridin-4-yl)-phenyl]-ethyl}-methyl-amine

Trifluoroacetic acid (3.5 ml) was added dropwise to a solution of {2-(4-chloro-phenyl)-2-[4-(1H-pyrrolo[2,3-b]pyridin-4-yl)-phenyl]-ethyl}-methyl-carbamic acid tert-butyl ester (49 mg, 0.11 mmol) in dichloromethane (3.5 ml), cooled in an ice bath. The reaction was allowed to stir at room temperature for 90 minutes. After this period the solvents were concentrated. Purification on SCX-II acid resin, eluting with methanol, then 2M ammonia/methanol, gave {2-(4-chloro-phenyl)-2-[4-(1H-pyrrolo[2,3-b]pyridin-4-yl)-phenyl]-ethyl}-methyl-amine (33 mg, 0.09 mmol, 83%). LC-MS (LCT2) m/z 362 [M+H⁺], Rₜ 4.19 min.

### Example 31

### C-[1-(7H-Pyrrolo[2,3-d]pyrimidin-4-yl)piperidin-3-yl]methylamine

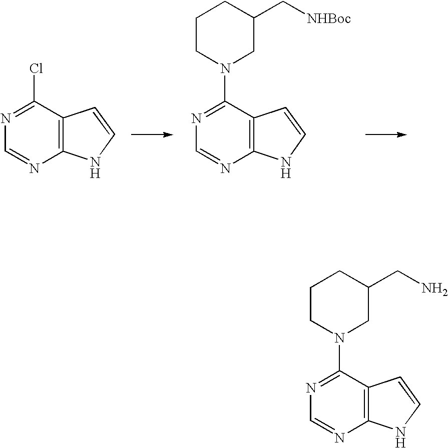

The title compound was prepared using the methods described for Example 18. LC-MS (LCT2) m/z 232 [M+H⁺], Rₜ 0.72 min.

### Example 32

### C-(4-Chlorophenyl)-C-[1-(1H-pyrrolo[2,3-b]pyridin-4-yl)piperidin-4-yl]methylamine

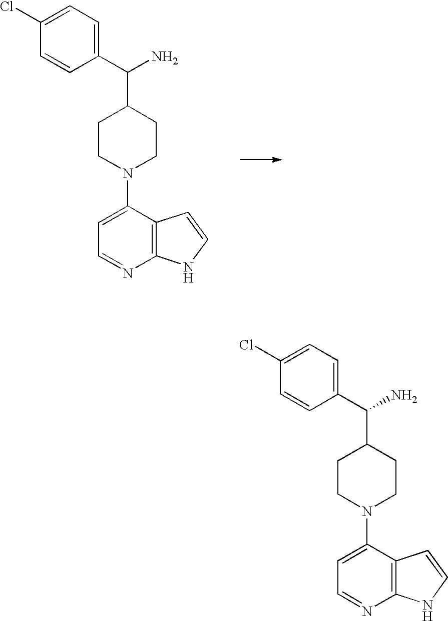

The title compound was prepared by separation of the enantiomers of the product of Example 29 by chiral HPLC using the Agilent chiral preparative conditions set out above. The retention time obtained using the Agilent chiral analytical conditions AS-CA was 33.7. LC/MS subsequently carried out using the PS-A conditions gave a retention time of 1.69 and an [M+H]+value of 341.

### Biological Activity

### Example 33

### Anti-Proliferative Activity

The anti-proliferative activities of compounds for use according to the invention are determined by measuring the ability of the compounds to inhibition of cell growth in a number of cell lines. Inhibition of cell growth is measured using the Alamar Blue assay (Nociari, M. M, Shalev, A., Benias, P., Russo, C. Journal of Immunological Methods 1998, 213, 157-167). The method is based on the ability of viable cells to reduce resazurin to its fluorescent product resorufin. For each proliferation assay cells are plated onto 96 well plates and allowed to recover for 16 hours prior to the addition of inhibitor compounds for a further 72 hours. At the end of the incubation period 10% (v/v) Alamar Blue is added and incubated for a further 6 hours prior to determination of fluorescent product at 535 nM ex/590 nM em. In the case of the non-proliferating cell assay cells are maintained at confluence for 96 hour prior to the addition of inhibitor compounds for a further 72 hours. The number of viable cells is determined by Alamar Blue assay as before. All cell lines are obtained from ECACC (European Collection of cell Cultures) or ATCC.

In particular, compounds for use according to the invention were tested against the PC3 cell line (ATCC Reference: CRL-1435) derived from human prostate adenocarcinoma. Preferred compounds for use according to the invention were found to have IC₅₀ values of less than 30 μM in this assay.

### Pharmaceutical Formulations

### Example 34

### (i) Tablet Formulation

A tablet composition containing a compound of the formula (I) is prepared by mixing 50 mg of the compound with 197 mg of lactose (BP) as diluent, and 3 mg magnesium stearate as a lubricant and compressing to form a tablet in known manner.

### (ii) Capsule Formulation

A capsule formulation is prepared by mixing 100 mg of a compound of the formula (I) with 100 mg lactose and filling the resulting mixture into standard opaque hard gelatin capsules.

### (iii) Injectable Formulation I

A parenteral composition for administration by injection can be prepared by dissolving a compound of the formula (I) (e.g. in a salt form) in water containing 10% propylene glycol to give a concentration of active compound of 1.5% by weight. The solution is then sterilised by filtration, filled into an ampoule and sealed.

### (iv) Injectable Formulation II

A parenteral composition for injection is prepared by dissolving in water a compound of the formula (I) (e.g. in salt form) (2 mg/ml) and mannitol (50 mg/ml), sterile filtering the solution and filling into sealable 1 ml vials or ampoules.

### v) Injectable Formulation III

A formulation for i.v. delivery by injection or infusion can be prepared by dissolving the compound of formula (I) (e.g. in a salt form) in water at 20 mg/ml. The vial is then sealed and sterilised by autoclaving.

### vi) Injectable Formulation IV

A formulation for i.v. delivery by injection or infusion can be prepared by dissolving the compound of formula (I) (e.g. in a salt form) in water containing a buffer (e.g. 0.2 M acetate pH 4.6) at 20 mg/ml. The vial is then sealed and sterilised by autoclaving.

### (vii) Subcutaneous Injection Formulation

A composition for sub-cutaneous administration is prepared by mixing a compound of the formula (I) with pharmaceutical grade corn oil to give a concentration of 5 mg/ml. The composition is sterilised and filled into a suitable container.

### viii) Lyophilised Formulation

Aliquots of formulated compound of formula (I) are put into 50 ml vials and lyophilized. During lyophilisation, the compositions are frozen using a one-step freezing protocol at (−45° C.). The temperature is raised to −10° C. for annealing, then lowered to freezing at −45° C., followed by primary drying at +25° C. for approximately 3400 minutes, followed by a secondary drying with increased steps if temperature to 50° C. The pressure during primary and secondary drying is set at 80 millitor.

### Example 35

### ROCK-II (h) Assay Protocol

In a final reaction volume of 25 μl, ROCK-II (h) (5-10 mU) is incubated with 50 mM Tris pH 7.5, 0.1 mM EGTA, 30 μM KEAKEKRQEQIAKRRRLSSLRASTSKSGGSQK, 10 mM MgAcetate and [γ-³³P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

### Example 36

### Anti-ROCK-II Activity

The compound of example 29 was tested for anti-ROCK-II activity (assay as described above):

| Example No. | IC₅₀ (μM) |
|---|---|
| 29 | <0.1 |

Thus, the compound tested exhibited inhibitory activity against ROCK-II.

### Example 37

### P70s6 Radiometric Assay

### Overview

P70S6 enzyme is bought from Upstate and used at 2 nM in the assay.

The substrate S6 cocktail (AKRRRLSSLRA) is used at 25 μM (Km has not been determined). In the phosphoryl transfer reaction, the ³³P-γ phosphate from ATP is transferred to the serine residue. The reaction mixture is transferred to a phosphocellulose filter plate where the peptide binds and the unused ATP is washed away. After washing, scintillant is added and the incorporated activity measured by scintillation counting.

### Reagents

P70S6 kinase (T412E) active from Upstate (#14-486)

S6 kinase substrate cocktail from Upstate (#20-122)

| | |
|---|---|
| Assay Buffer | 10 mM MOPS pH 7.0 |
| | 0.1 mg/ml BSA |
| | 0.001% Brij-35 |
| | 0.5% glycerol |
| | 0.2 mM EDTA |
| | 10 mM MgCl₂ |
| | 0.01% β-mercaptoethanol |
| | Made as a 10X stock, stored at 20° C. in 2 ml aliquots |
| | 15 μM ATP |

ATP (10 mM stock) added fresh from concentrated stocks.

ATP will break down over time, keep on ice as far as possible and use small aliquots to ensure the stock is fresh.

γ³³P-ATP APBiotech (BF1000)

12.5% orthophosphoric acid

0.5% orthophosphoric acid

Microscint 20 (Packard)

### Assay Preparation

### Enzyme Mix (per 1 ml -100 Assay Points):

743.75 μl H20

250 μl 10× assay buffer

3.75 μl 10 mM ATP

2.5 μl enzyme

Substrate Mix (per 1 ml -100 Assay Points):

250 μl S6 cocktail substrate

750 μl H20

3.5 μl ³³P-ATP (BF₁₀₀₀ from APBiotech)

The amount of ³³P-ATP added assumes it is on its reference date. The exact amount needs to be adjusted with time.

Compounds—prepare a dilution curve in DMSO in a polypropylene 96 well plate to 40× final assay concentration (final DMSO 2.5%).

Dilute 1:8 in water (adding 5 μl of compound to 35 μl water is sufficient).

### Assay Setup

In a polypropylene 96 well plate add in order:
5 μl compound
10 μl substrate mix
10 μl enzyme mix

Final ATP concentration is approximately 15 μM. KM for ATP calculated to 47 uM radiometrically. Controls are “no compound” (DMSO only) and “no enzyme” (use 10 μl of the enzyme mix prior to adding enzyme). Cover with a plate seal (TopSeal A—Packard) or plastic lid from filter plate (moderate radiation barrier). Mix by gentle shaking. Incubate at room temperature for 50 minutes. Stop the reaction by adding 20 μl of 2% orthophosphoric acid.

### Filtration Step

Pre-wet the wells of a Millipore MAPH NOB plate with 50 μl of 0.5% orthophosphoric acid wash buffer. Filter the liquid through on a Millipore vacuum filtration unit. Transfer the whole of the stopped reaction to the wells. Filter through. Wash twice with 200 μl of 0.5% orthophosphoric acid wash buffer. Vacuum to near dryness. Remove the plate support and allow to the filters to dry further on tissue paper. Snap the plate into an adapter for the Packard TopCount. Add 20 μl of Microscint 20 scintillant, seal with a sheet of Topseal A and count for 30s on the TopCount.

### EQUIVALENTS

The foregoing examples are presented for the purpose of illustrating the invention and should not be construed as imposing any limitation on the scope of the invention. It will readily be apparent that numerous modifications and alterations may be made to the specific embodiments of the invention described above and illustrated in the examples without departing from the principles underlying the invention. All such modifications and alterations are intended to be embraced by this application.

1-68. (canceled)

69. A method for (a) the treatment or prophylaxis of a disease or condition in which the modulation of ROCK kinase or protein kinase p70S6K is indicated; and/or (b) the treatment of a subject or patient population in which the modulation of ROCK kinase or protein kinase p70S6K is indicated; which method comprises administering to a subject in need thereof, an effective therapeutic amount of a compound of the formula (I): 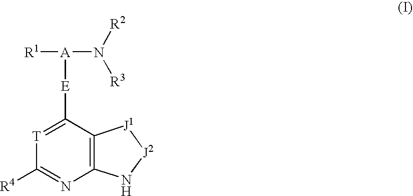
or salts, solvates, tautomers or N-oxides thereof, wherein
T is N or a group CR⁵;
J¹-J² represents a group selected from N═C(R⁶), (R⁷)C═N, (R⁸)N—C(O), (R⁸)₂C—C(O), N═N and (R⁷)C═C(R⁶);
A is a saturated hydrocarbon linker group containing from 1 to 7 carbon atoms, the linker group having a maximum chain length of 5 atoms extending between R¹ and NR²R³ and a maximum chain length of 4 atoms extending between E and NR²R³, wherein one of the carbon atoms in the linker group may optionally be replaced by an oxygen or nitrogen atom; and wherein the carbon atoms of the linker group A may optionally bear one or more substituents selected from oxo, fluorine and hydroxy, provided that the hydroxy group when present is not located at a carbon atom ca with respect to the NR²R³ group and provided that the oxo group when present is located at a carbon atom ca with respect to the NR²R³ group;
E is a monocyclic or bicyclic carbocyclic or heterocyclic group or an acyclic group X-G wherein X is selected from CH₂, O, S and NH and G is a C₁₋₄ alkylene chain wherein one of the carbon atoms is optionally replaced by O, S or NH;
R¹ is hydrogen or an aryl or heteroaryl group;
R² and R³ are independently selected from hydrogen, C₁₋₄ hydrocarbyl and C₁₋₄ acyl wherein the hydrocarbyl and acyl groups are optionally substituted by one or more substituents selected from fluorine, hydroxy, amino, methylamino, dimethylamino, methoxy and a monocyclic or bicyclic aryl or heteroaryl group;
or R² and R³ together with the nitrogen atom to which they are attached form a cyclic group selected from an imidazole group and a saturated monocyclic heterocyclic group having 4-7 ring members and optionally containing a second heteroatom ring member selected from O and N;
or one of R² and R³ together with the nitrogen atom to which they are attached and one or more atoms from the linker group A form a saturated monocyclic heterocyclic group having 4-7 ring members and optionally containing a second heteroatom ring member selected from O and N. the monocyclic heterocyclic group being optionally substituted by one or more C₁₋₄ alkyl groups;
or NR²R³ and the carbon atom of linker group A to which it is attached together form a cyano group; or
R¹, A and NR²R³ together form a cyano group; and
R⁴, R⁵, R⁶, R⁷ and R⁸ are each independently selected from hydrogen; halogen; C₁₋₆ hydrocarbyl optionally substituted by halogen, hydroxy or C₁₋₂ alkoxy; cyano; CONH₂; CONHR⁹; CF₃; NH₂; NHCOR⁹ and NHCONHR⁹;
R⁹ is phenyl or benzyl each optionally substituted by one or substituents selected from halogen, hydroxy, trifluoromethyl, cyano, nitro, carboxy, amino, mono- or di-C₁₋₄ hydrocarbylamino; a group R^{a}-R^{b} wherein R^{W} is a bond, O, CO, X¹C(X²), C(X²)X¹, X¹C(X²)X¹, S. SO, SO₂, NR^{c}, SO₂NR^{c} or NR^{c}SO₂; and R^{b} is selected from hydrogen, heterocyclic groups having from 3 to 12 ring members, and a C₁₋₈ hydrocarbyl group optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹;
R^{c} is selected from hydrogen and C₁₋₄ hydrocarbyl; and
X¹ is O, S or NR^{c} and X² is ═O, ═S or ═NR^{c}.

70. A method according to claim 69 wherein:
R² and R³ are independently selected from hydrogen, C₁₋₄ hydrocarbyl and C₁₋₄ acyl;
or R² and R³ together with the nitrogen atom to which they are attached form a saturated monocyclic heterocyclic group having 4-7 ring members and optionally containing a second heteroatom ring member selected from O and N. the monocyclic heterocyclic group being optionally substituted by one or more C₁₋₄ alkyl groups;
or one of R² and R³ together with the nitrogen atom to which they are attached and one or more atoms from the linker group A form a saturated monocyclic heterocyclic group having 4-7 ring members and optionally containing a second heteroatom ring member selected from O and N. the monocyclic heterocyclic group being optionally substituted by one or more C₁₋₄ alkyl groups;
or NR²R³ and the carbon atom of linker group A to which it is attached together form a cyano group; or
R¹, A and NR²R³ together form a cyano group.

71. A method according to claim 69 wherein the compounds of formula (I) is as defined in claim 1 provided that:
(a-i) when J¹-J² is (R⁷)C═C(R⁶) and E is a monocyclic or bicyclic group linked through a nitrogen atom to the ring containing T. then A contains no oxo substituent;
(a-ii) E is other than an unsubstituted or substituted indole group;
(a-iii) when J¹-J² is N═CH, then E-A(R¹)—NR²R³ is other than a group —S—(CH₂)₃—CONH₂ or —S—(CH₂)₃—CN;
(a-iv) when J¹-J² is CH═N, then E-A(R¹)—NR²R³ is other than a group —NH—(CH₂)ₙ—N(CH₂CH₃)₂ where n is 2 or 3; and
(a-v) when J¹-J² is N═CH, then E-A(R¹)—NR²R³ is other than a group —NH—(CH₂)₂—NH₂ or —NH—(CH₂)₂—N(CH₃)₂.

72. A method according to claim 69 wherein:
T is N or a group CR⁵;
J¹-J² represents a group selected from N═C(R⁶), (R⁷)C═N, (R⁸)N—C(O), (R⁸)₂C—C(O), N═N and (R⁷)C═C(R⁶);
A is a saturated hydrocarbon linker group containing from 1 to 7 carbon atoms, the linker group having a maximum chain length of 5 atoms extending between R¹ and NR²R³ and a maximum chain length of 4 atoms extending between E and NR²R³, wherein one of the carbon atoms in the linker group may optionally be replaced by an oxygen or nitrogen atom; and wherein the carbon atoms of the linker group A may optionally bear one or more substituents selected from fluorine and hydroxy, provided that the hydroxy group when present is not located at a carbon atom ca with respect to the NR²R³ group;
E is a monocyclic carbocyclic or heterocyclic group;
R¹ is an aryl or heteroaryl group;
R² and R³ are independently selected from hydrogen, C₁₋₄ hydrocarbyl and C₁₋₄ acyl wherein the hydrocarbyl and acyl groups are optionally substituted by one or more substituents selected from fluorine, hydroxy, amino, methylamino, dimethylamino, methoxy and a monocyclic or bicyclic aryl or heteroaryl group;
or R² and R³ together with the nitrogen atom to which they are attached form a saturated monocyclic heterocyclic group having 4-7 ring members and optionally containing a second heteroatom ring member selected from O and N;
or one of R² and R³ together with the nitrogen atom to which they are attached and one or more atoms from the linker group A form a saturated monocyclic heterocyclic group having 4-7 ring members and optionally containing a second heteroatom ring member selected from O and N. the monocyclic heterocyclic group being optionally substituted by one or more C₁₋₄ alkyl groups;
or NR²R³ and the carbon atom of linker group A to which it is attached together form a cyano group; or
R¹, A and NR²R³ together form a cyano group; and
R⁴, R⁵, R⁶, R⁷ and R⁸ are each independently selected from hydrogen; halogen; C₁₋₆ hydrocarbyl optionally substituted by halogen, hydroxy or C₁₋₂ alkoxy; cyano; CONH₂; CONHR⁹; CF₃; NH₂; NHCOR⁹ and NHCONHR⁹;
R⁹ is phenyl or benzyl each optionally substituted by one or substituents selected from halogen, hydroxy, trifluoromethyl, cyano, nitro, carboxy, amino, mono- or di-C₁₋₄ hydrocarbylamino; a group R^{a}-R^{b} wherein R^{a} is a bond, O, CO, X¹C(X²), C(X²)X¹, X¹C(X²)X¹, S, SO, SO₂, NR^{c}, SO₂NR^{c} or NR^{c}SO₂; and R^{b} is selected from hydrogen, heterocyclic groups having from 3 to 12 ring members, and a C₁₋₈ hydrocarbyl group optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹;
R^{c} is selected from hydrogen and C₁₋₄ hydrocarbyl; and
X¹ is O, S or NR^{c} and X² is ═O, ═S or ═NR^{c}.

73. A method according to claim 72 wherein the monocyclic carbocyclic or heterocyclic group E is selected from phenyl, thiophene, furan, pyrimidine, pyrazine, pyridine, cyclohexane, cyclopentane, piperidine, piperazine and piperazine groups, and E is unsubstituted or has up to 4 substituents R¹¹ selected from hydroxy; CH₂CN, oxo (when E is non-aromatic); halogen (e.g. chlorine and bromine); trifluoromethyl; cyano; C₁₋₄ hydrocarbyloxy optionally substituted by C₁₋₂ alkoxy or hydroxy; and C₁₋₄ hydrocarbyl optionally substituted by C₁₋₂ alkoxy or hydroxy.

74. A method according to claim 73 wherein E is selected from phenyl and piperidine groups.

75. A method according to claim 73 wherein E is unsubstituted.

76. A method according to claim 69 wherein R⁴ is selected from hydrogen, chlorine, fluorine and methyl.

77. A method according to claim 69 wherein T is N or CR⁵ wherein R⁵ is hydrogen.

78. A method according to claim 69 wherein R⁶ is selected from hydrogen, chlorine, fluorine and methyl; R⁷ is selected from hydrogen, chlorine, fluorine and methyl; and R⁸ is selected from hydrogen, chlorine, fluorine and methyl.

79. A method according to claim 69 wherein the linker group A has a maximum chain length of 3 atoms extending between R¹ and NR²R³, and a maximum chain length of 4 atoms extending between E and NR²R³.

80. A method according to claim 79 wherein the linker group A has an all-carbon skeleton.

81. A method according to claim 69 wherein R¹ is an aryl or heteroaryl group selected from unsubstituted or substituted phenyl, naphthyl, thienyl, furan, pyrimidine and pyridine groups.

82. A method according to claim 81 wherein R¹ is unsubstituted or substituted by up to 5 substituents selected from hydroxy; C₁₋₄ acyloxy; fluorine; chlorine; bromine; trifluoromethyl; cyano; C₁₋₄ hydrocarbyloxy and C₁₋₄ hydrocarbyl each optionally substituted by C₁₋₂ alkoxy or hydroxy.

83. A method according to claim 69 wherein R² and R³ are independently selected from hydrogen, unsubstituted C₁₋₄ hydrocarbyl and unsubstituted C₁₋₄ acyl.

84. A method according to claim 83 wherein R² and R³ are independently selected from hydrogen and methyl.

85. A method according to claim 69 wherein J¹-J² is selected from N═CH, HC═N, HN—C(O) and CH═CH.

86. A method according to claim 73 wherein the compound of formula (I) is represented by the formula (II): 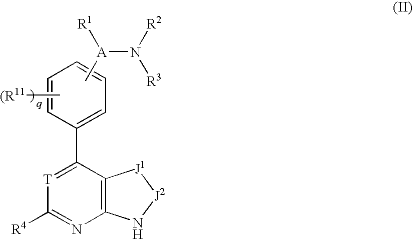
or salts, solvates, tautomers or N-oxides thereof, wherein the group A is attached to the meta or para position of the benzene ring and q is 0-4.

87. A method according to claim 73 wherein the compound of formula (I) is represented by the formula (III): 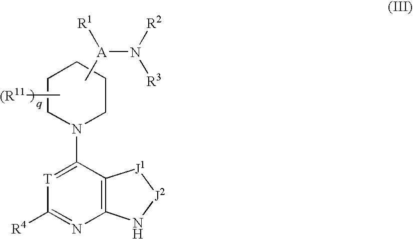
or salts, solvates, tautomers or N-oxides thereof, wherein the group A is attached to the 3-position or 4-position of the piperidine ring, and q is 0-4.

88. A method according to claim 69 wherein the disease or condition is selected from: (a) tumour metastasis; (b) tumour invasion; (c) tumour progression; (d) tumour adhesion; (e) actinomycin contractility-dependent tumour metastasis, invasion or progression; (f) cell transformation; (g) ROCK-mediated tumour metastasis, invasion, progression or adhesion; (h) ROCK-mediated actinomycin contractility-dependent tumour metastasis, invasion or progression; (i) ROCK-mediated cell transformation; (j) cancer; (k) ROCK-mediated cancer.
